(19) United States
(12) Patent Application Publication
(10) Pub. No.: US 2023/0017312 A1
(43) Pub. Date: Jan. 19, 2023

(54) CENTRALLY ACTIVE P38ALPHA INHIBITING COMPOUNDS

(71) Applicant: Synovo GmbH, Tübingen (DE)

(72) Inventors: Stefan Laufer, Tübingen (DE); Wolfgang Albrecht, Tübingen (DE); Pierre Koch, Altensteig (DE); Niklas Walter, Tübingen (DE)

(73) Assignee: Synovo GmbH, Tübingen (DE)

(21) Appl. No.: 17/638,751

(22) PCT Filed: Aug. 26, 2020
(86) PCT No.: PCT/IB2020/000693

§ 371 Date: Feb. 25, 2022

Related U.S. Application Data
(60) Provisional application No. 62/892,346, filed on Aug. 27, 2019.

(51) Int. Cl. C07D 313/12; C07D 413/10; C07D 413/12; C07C 49/115; C07C 49/747

(52) U.S. Cl. CPC C07D 313/12; C07D 413/10; C07D 413/12; C07C 49/115; C07C 49/747

(57) ABSTRACT

Compounds that are inhibitors of p38alpha and centrally available are described.

### BACKGROUND

Inhibitors of p38 kinase have broad utility in pharmacy, however, to date, most have been designed to be active in peripheral sites in the body such as the joints. The rationale was that they should be useful in rheumatic diseases. We and others have determined that substances that inhibit p38 alpha may also be useful in the brain and in particular in diseases such as Alzheimer's disease. We have designed compounds to be available to the central compartment and to be active in inflammatory and degenerative diseases of the brain based on the tricyclic “Skepinone” system (see Köberle et al., 2011).

### SUMMARY OF THE INVENTION

In some embodiments, the compounds have the following formulae (including any possible salts, solvates, hydrates thereof, and any structures with exchanged isotopes, as possible by state of the art):

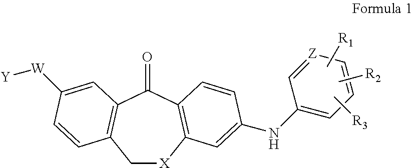

W=bond*, —C(═O)—

X=O, CH₂

Y=OR₄, NR₉R₄

Z=N, C(R₁)

R₁, R₂, R₃=independent of each other H, F, Cl, Br, I, NH₂, NHCH₃, N(CH₃)₂, NO₂

R₄=H, OH, phenyl, C₁-C₁₀-alkyl, linear or cyclic, branched or unbranched; optionally substituted with 1 to 6 substituents of the group: F, OH, OR₆, SH, SCH₃, NH₂, NHR₆, NR₆R₇, COOH, COOCH₃, 1-morpholinyl, 1-piperidinyl, 1-(4-R₈)piperazinyl, 3-(1H)indolyl, 4-(1H)imidazolyl, phenyl (optionally substituted with OH, OCH₃, F, Cl, Br, I, N(R₉)₂);

or C₁-C₈-alkyl as described above and 1 or 2 links of the carbon chain replaced by O, NH, NR₆;
or 2-(2-oxa-6-azaspiro[3,3]heptan-6-yl)ethyl;

R₆, R₇=independent of each other C₁-C₂-alkyl, optionally substituted with OH, OCH₃, NH₂, NHCH₃, N(CH₃)₂, COOH, COOCH₃, 1-Morpholinyl, 1-piperidinyl, 1-(4-R₈)piperazinyl, phenyl;

R₈=H, CH₃, Boc, Fmoc, Z;

R₉=H, Me;

or R₄, R₉=—CH₂—(V)ₙ—CH₂—, V=CH₂, S, O; n=1-4; and

* “bond” indicates the direct connection between Y and the aromatic ring.

In one aspect, the invention provides a composition comprising a compound the formulae herein, or salt, solvate, hydrate or prodrug thereof, and a pharmaceutically acceptable carrier. In a further aspect, the composition can further comprise an additional therapeutic agent.

In one aspect, the invention provides a method of treating a subject suffering from or susceptible to a disease, disorder, or symptom thereof. The method includes administering to a subject in need thereof a therapeutically effective amount of a compound of any of the formulae herein, or salt, solvate, hydrate or prodrug thereof. The disease, disorder, or symptom thereof can be, for example, Alzheimer's disease or other degenerative diseases of the brain.

### DETAILED DESCRIPTION OF THE INVENTION

### Technical Problem

Most kinase inhibitors and p38 inhibitors in particular have been selected for properties that exclude them from the brain for reasons of simplifying pharmaceutical development and increasing safety in long term therapy. This meant that despite potential utility, most known substances were not practically useful for diseases of the brain. We provide inhibitors with central activity.

### Solution to Problem

Tricyclic derivatives with sidechains suitable for brain uptake and potent anti-p38 activity.

### Advantageous Effects of Invention

The compounds are available to the brain at low systemic doses while having low nM inhibition of p38alpha.

### Description of Embodiments

The compounds described in this section can be prepared by methods known in the art, as well as by the synthetic routes disclosed herein. Detailed routes including various intermediates are illustrated in the examples herein.

The chemicals used may include, for example, solvents, reagents, catalysts, protecting group and deprotecting group reagents and the like. The methods described may also additionally comprise steps, either before or after the steps described specifically herein, to add or remove suitable protecting groups in order to ultimately allow synthesis of the compound of the formulae described herein.

As can be appreciated by the skilled artisan, the synthetic routes herein are not intended to comprise a comprehensive list of all means by which the compounds described and claimed in this application may be synthesized. Further methods will be evident to those of ordinary skill in the art. Additionally, the various synthetic steps described above may be performed in an alternate sequence or order to give the desired compounds.

In one aspect, these compounds are suitable for use as medicaments and in particular for the treatment of degenerative diseases of the brain such as Alzheimer's disease.

In an embodiment, the compound of the invention is administered to the subject using a pharmaceutically-acceptable formulation, e.g., a pharmaceutically-acceptable formulation that provides sustained delivery of the compound of the invention to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to those compound of the inventions of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention.

Some examples of substances which can serve as pharmaceutical carriers are sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, anti-oxidants and preservatives, can also be present. Solubilizing agents, including for example, cremaphore and beta-cyclodextrins can also be used in the pharmaceutical compositions herein.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

Initial dosages also can be estimated from in vivo data, such as animal models. Animal models useful for testing the efficacy of compounds to treat or prevent the various diseases described above are well-known in the art.

Dosage amounts will typically be in the range of from about 0.0001 or 0.001 or 0.01 mg/kg/day to about 100 mg/kg/day, but can be higher or lower, depending upon, among other factors, the activity of the compound, its bioavailability, the mode of administration, and various factors discussed above. Dosage amount and interval can be adjusted individually to provide plasma levels of the compound(s) which are sufficient to maintain therapeutic or prophylactic effect. In cases of local administration or selective uptake, such as local topical administration, the effective local concentration of active compound(s) cannot be related to plasma concentration. Skilled artisans will be able to optimize effective local dosages without undue experimentation.

The compound(s) can be administered once per day, a few or several times per day, or even multiple times per day, depending upon, among other things, the indication being treated and the judgment of the prescribing physician.

Preferably, the compound(s) will provide therapeutic or prophylactic benefit without causing substantial toxicity. Toxicity of the compound(s) can be determined using standard pharmaceutical procedures. The dose ratio between toxic and therapeutic (or prophylactic) effect is the therapeutic index. Compounds(s) that exhibit high therapeutic indices are preferred.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

Another object of the present invention is the use of a compound as described herein (e.g., of any formulae herein) in the manufacture of a medicament for use in the treatment of a disorder or disease herein. Another object of the present invention is the use of a compound as described herein (e.g., of any formulae herein) for use in the treatment of a disorder or disease herein.

Many compounds of this invention have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., treating a disease).

### Examples

Unless otherwise specified, all commercially available reagents and solvents were used without prior purification. All chemical structures and names are generated from ChemDraw Ultra (Cambridge). Flash column chromatography was carried out using Interchim PuriFlash 430 Flash Chromatography System on silica columns unless otherwise mentioned. NMR spectra were measured either on a Bruker Avance 200 or Bruker Avance 400 using the appropriate deuterated solvents. Purity for all final compounds was confirmed to be greater than 95%, unless otherwise stated, by HPLC using a Hewlett-Packard HP 1090 Series II LC and an Agilent 1100 Series equipped with an UV Diodenarray-Detector (DAD, Detection at 230 nm and 254 nm).

### General Method/Procedure

### Method A—Esterification

### Variant A

The carboxylic moiety was dissolved in an excess amount of the corresponding alcohol which was then heated to reflux. A catalytic amount of concentrated H₂SO₄ was carefully added. Reaction was monitored to completion. After cooling the reaction, the excess alcohol was removed under reduced pressure. The residue was taken up in EtOAc, washed with a dilute NaOH solution, dried over Na₂SO₄ and evaporated under reduced pressure producing solid product.

### Variant B

The amino acid was taken up in 5 mL of the corresponding alcohol and cooled in an ice-bath. Thionyl chloride (5 eq.) was added carefully via syringe along the edge of the flask. The reaction was stirred until completion. The reaction was evaporated under reduced pressure to dryness and the corresponding HCl salt of the product is obtained as white solid.

### Variant C

The carboxylic acid moiety was dissolved in 5 mL DMF with heating (50° C.) under an inert gas. To this was added, CDI (2 eq) and stirred for 2 h. The corresponding alcohol (1.5 eq) was added to the reaction and stirred for 1 h (or until completion) at 50° C. At this point, H₂O (10 mL) was then added. This was extracted with either EtOAc or DCM (3×). The combined organic phases were dried over Na₂SO₄, filtered and evaporated under reduced pressure. The product, when needed, could be purified by Flash Chromatography.

### Variant D

The carboxylic acid moiety was dissolved in 5 mL DMF with heating (50° C.) under an inert gas. To this was added DIPEA (2.5 eq) and TBTU (1.5 eq). The reaction was activated for 30 min. The corresponding alcohol (3 eq) was added and the reaction stirred for 1 h or until completion at 50° C. At this point, H₂O (10 mL) was then added. This was extracted with either EtOAc or DCM (3×). The combined organic phases were dried over Na₂SO₄, filtered and evaporated under reduced pressure. The product, when needed, could be purified by Flash Chromatography.

### Method B—Saponification

The ester was taken up in methanol and KOH (2.5 eq) was added neat. The reaction was heated under reflux until completion and cooled to room temperature. The solvent was removed under pressure and the residue taken up in EtOAc. The organic phase was washed with a diluted HCl solution, dried over Na₂SO₄, and the solvent evaporated under reduced pressure obtaining a solid product. The product, when needed, could be purified by Flash Chromatography.

### Method C—Amide Coupling

### Variant A

Sodium hydride was suspended in THF (5 mL) and DMF (5 mL). The aniline was taken up in THF (10 mL) and added dropwise to the sodium hydride suspension under argon. The reaction was stirred for 2 h at room temperature until no more gas evolution was observed and the reaction turned into a clear solution. The reaction solution was cooled in an ice bath to 0° C. and the solution of acid chloride (1 eq) in THF (3 mL) was slowly added over 15 min. The reaction was stirred overnight at room temperature until completion and neutralized with H₂O. This was extracted with EtOAc (3×50 mL). The combined organics were dried over Na₂SO₄, filtered and evaporated under reduced pressure to dryness. The product was purified by flash chromatography.

### Variant B

The carboxylic acid moiety was dissolved in 5 mL DMF with heating (50° C.) under an inert gas. To this was added, CDI (2 eq) and stirred for 2 h. The corresponding amine (1.5 to 5 eq) was added to the reaction. This was stirred for 1 h at 50° C. until completion. At this point, H₂O (10 mL) was then added. This was extracted with either EtOAc or DCM (3×). The combined organic phases were dried over Na₂SO₄, filtered and evaporated under reduced pressure. The product, when needed, could be purified by Flash Chromatography.

### Variant C

The carboxylic acid moiety was dissolved in 5 mL DMF with heating (50° C.) under an inert gas. To this was added DIPEA (2.5 eq) and TBTU (1.5 eq). The reaction was activated for 15 min. The corresponding amine (1.2 to 5 eq) was added and the reaction stirred for 1 h or until completion at 50° C. At this point, H₂O (10 mL) was then added. This was extracted with either EtOAc or DCM (3×). When the coupling is with an amino acid (ester), the organics were washed with 2N KHSO₄ solution (2×). The combined organic phases were dried over Na₂SO₄, filtered and evaporated under reduced pressure. The product, when needed, could be purified by Flash Chromatography.

### Method D—Friedel Crafts Acylation

### Variant A

For the syntheses of cyclic ketones, the 50° C. carboxylic acid was suspended in DCM in a 3-necked round bottom flask under argon and the suspension was heated under reflux. To this was added thionyl chloride (5.5 eq) dropwise. Heating was continued until reaction becomes a clear solution. The reaction was cooled to around 50° C. At this point, anhydrous AlCl₃ (5.5 eq) was added and the solution slowly heated once again to reflux. After reaction completion, the reaction was poured into ice-water and stirred for 30 min. The organic phase was washed with diluted HCl solution, dried over Na₂SO₄, and evaporated under reduced pressure to dryness to obtain a solid.

### Variant B

The substituted benzoyl ester was weighed into a 25 mL-round bottom flask. To this was added, under argon, thionyl chloride (5 mL). The reaction was heated for 2 h under reflux, cooled to room temperature and the excess thionyl chloride removed under reduced pressure over a cold trap. The acid chloride formed was used directly for the next step.

In a 100-mL 3-necked round bottom flask was transferred dry AlCl₃ (2.5 eq) under argon and ice-cooling in benzene (20 mL). To the resulting suspension was carefully added, the acid chloride over the edge of the flask. The reaction mixture was stirred for 3 h at 0° C. At this point, the reaction was terminated by addition of ice, and after 30 min stirring, the reaction mixture was extracted with DCM (3×30 mL). The combined organics were evaporated under reduced pressure to dryness to obtain the nitrobenzophenone as yellow solid.

### Method E—Buchwald-Hartwig Coupling

### Variant A

For the production of the secondary amine, the aniline (1.2 eq) was transferred to a dry 50-mL 3-necked round bottom flask under argon. This was taken up in a 1,4-dioxane/t-BuOH mixture (5:1, v/v) and dissolved with warming. To this was added successively, XPhos (0.025 eq), Cs₂CO₃ (1.5 eq), Pd(OAc)₂ (0.05 eq) and the acid chloride. The reaction mixture was heated for 1 h to 110° C. under argon. Reaction progress was monitored by TLC. After reaction completion, the reaction was cooled to room temperature and filtered. The residue was washed several times with DCM, MeOH and EtOAc. The combined organics were evaporated under reduced pressure to dryness. The dark brown product was purified by flash chromatography.

### Variant B

For the production of the secondary amine, the aniline (1.2 eq), XPhos-Pd 2. Gen (0.03 eq) and the acid chloride was transferred to a dry 50-mL 3-necked round bottom flask under argon. This was taken up in a 1,4-dioxane/t-BuOH mixture (5:1, v/v) and dissolved with warming. To this was added, Cs₂CO₃ (1.5 eq). The reaction mixture was heated to 60° C. under argon. Reaction progress was monitored by TLC. After completion, the reaction was cooled to room temperature and filtered. The residue was washed several times with DCM, MeOH and EtOAc. The combined organics were evaporated under reduced pressure to dryness. The dark brown product was purified by flash chromatography.

### Method F—Nitration

The corresponding substituted aniline was dissolved in concentrated H₂SO₄ (25 mL) and cooled to −10° C. The reaction flask was equipped with a dropping funnel and concentrated nitric acid (5 mL) was introduced dropwise to the reaction. The reaction was stirred until completion. The product mixture was poured into ice-water and the pH adjusted to pH 13 making sure that the temperature does not go over 80° C. (ice-cooling). After cooling to room temperature, the aqueous phase was extracted with Et₂O (3×50 mL). The combined organics were dried over Na₂SO₄, filtered and evaporated under reduced pressure to dryness. The product, if necessary, was purified by flash chromatography.

### Method G—Reduction of the Nitro Group

The nitro compound is dissolved in EtOH (40 mL) with heating (50° C.). SnCl₂ (3 eq) was added in portion and the reaction heated to reflux. After completion, the reaction was cooled to room temperature and NaHCO₃ was carefully added. After stirring for 15 min, EtOH was removed under reduced pressure. The residue was washed several times with EtOAc. The filtrate was evaporated under reduced pressure to dryness and the corresponding aniline was obtained as solid material. The produce could be purified with flash chromatography when needed.

### Method H—Nucleophilic Aromatic Substitution

The desired amount of aniline and 1-fluoro-2-nitrobenzen (1.1 eq) were transferred to a round bottom flask, dissolved in DMSO (10 mL) and the solution cooled in an ice bath. A solution of KOtBu (2 eq) in DMSO (5 mL) was added dropwise. The ice-bath was removed and the reaction was allowed to warm to room temperature where it was stirred for 18 h. After reaction completion, the reaction was terminated by the addition of H₂O. At pH 8, the reaction was extracted with EtOAc (3×50 mL). The combined organics were washed with H₂O and saturated aq. NaCl solution successively, dried over Na₂SO₄, filtered, and evaporated under reduced pressure to dryness. The crude product was purified by flash chromatography.

### Method I—Removal of Boc-Protection

### Variant A

The BOC-protected compound was taken up in dry DCM (5 mL) and to this was added, TFA (1.5 mL). Reaction was stirred until completion upon which a saturated aq. NaHCO₃ solution was added. The reaction was extracted with DCM (3×). The combined DCM extracts were removed in vacuo and the product purified by flash chromatography.

### Variant B

The BOC-protected amino acid was dissolved in methanol and cooled in an ice-bath. Oxalyl chloride (1.5 eq) was slowly added over the edge of the flask and the reaction vessel sealed air-tight. After 4 h, another batch of oxalyl chloride (1.5 eq.) was added. The reaction was stirred for an additional 8 h. The solvent was removed under reduced pressure and the crude product was purified over silica gel.

### Method J—Removal of Fmoc-Protection

The corresponding compound to be deprotected was taken up in dry DCM (when necessary, mixture needs to be heated to dissolve the substance). To this was added, piperidine (10% to 20%) slowly and in portions. The reaction was stirred until complete deprotection. After removal of the solvent under reduced pressure, the crude product was obtained.

### Procedures

### Example 1. 3-((2,4-Difluorophenyl)amino)-11-oxo-6,11-dihydrobenzo[b,e]oxepin-9-carboxylmethylester (15a)

### 4-methylisopthalic acid (2)

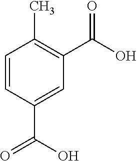

3-Bromo-4-methylbenzoic acid, 1, (10 g; 46.50 mmol) was weighed into a 250-mL 3-necked round bottom flask and taken up in dry THF (50 mL) under argon atmosphere. Using a cryostat, the reaction was cooled to 0° C. Under argon, 3M methyl magnesium bromide solution (17.7 mL; 51.15 mmol) in Et₂O was added dropwise. The reaction was stirred for 2 h. The reaction solution was then cooled to −75° C., and to this was added carefully, 2.5M n-BuLi solution in hexane (35.05 mL; 93.00 mmol) dropwise. The resulting reaction was stirred for 3 h and allowed to warm to −40° C. At this point, dry ice (25 g) was added to the reaction. Cooling was removed and the reaction stirred for an additional 3 h. When reaction was completed, the reaction was terminated by the addition of 20% NaOH. This was washed with EtOAc. The aqueous phase was cooled in an ice bath and was acidified by the addition concentrated HCl until the product precipitated out. The precipitate was filtered and washed with a very small amount of EtOAc producing the crude product as a white solid (7.32 g; 87.4% yield).

If the yield was too low, the EtOAc phase in the filtrate was separated and evaporated under reduced pressure. The residue was taken up in EtOAc and H₂O and then filtered.

¹H-NMR (200 MHz, DMSO-d6) δ 8.37 (d, J=1.8 Hz, 1H), 7.96 (dd, J=7.9, 1.9 Hz, 1H), 7.43 (d, J=8.1 Hz, 1H), 4.33 (s, 2H), 2.57 (s, 3H)

¹³C NMR (50 MHz, DMSO-d6) δ 168.71, 167.42, 145.13, 132.89, 131.96, 131.44, 129.35, 22.19.

MS (FAB_{neg}) (m/z): 179.0 [M−H]⁻

### 4-Methylisophthalic acid, dimethylester (3)

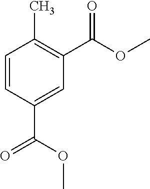

This was synthesized using general method A, variant A. Starting material: 4-methylisopthalic acid (2) (5.0 g; 27.75 mmol): Purification: flash chromatography, SiO₂, PE/EtOAc 6:1; white solid (5.21 g; 90.2% yield); mp. 76.4° C.

MS (FABₚₒₛ) (m/z): 209.1 [M+H]⁺

IR (ATR) [cm⁻¹] 3051, 2984, 2934, 2843, 1715, 1444, 1315, 1065, 995, 845, 757.

¹H-NMR (200 MHz, DMSO-d6) δ 8.18 (s, 1H), 7.84 (d, J=7.4 Hz, 1H), 7.33 (d, J=7.6 Hz, 1H), 3.81 (s, 6H), 2.58 (s, 3H).

¹³C NMR (50 MHz, DMSO-d6) δ 166.3, 165.40, 144.82, 132.33 (s, J=7.0 Hz), 132.26, 130.71, 129.70, 127.49, 52.19 (d, J=8.1 Hz), 21.13.

### 4-Bromomethylisophtalic acid, dimethylester (4)

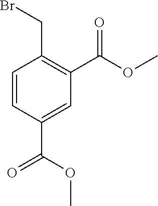 [SMILES: COC(=O)c1ccc(CBr)c(C(=O)OC)c1]

Compound 3 (2.50 g; 12.01 mmol) was weighed into a 100-mL 3-necked round bottom flask and dissolved in CCl₄ at 70° C. To this was added N-bromo succinimide and spatula-full AIBN. The reaction was heated to reflux for 4 h. After the reaction was complete, the reaction was cooled to room temperature and the organic phase was filtered to remove N-succinimide. The filtrate was evaporated under reduced pressure to obtain a yellow oil. Flash chromatography purification (SiO₂, PE/EtOAc 5:1) produced the product as an off-white, slowly crystallizing oil, 4 (3.11 g; 90.2%), mp 74.1° C.

MS (FABₚₒₛ) (m/z): 288.0 [M+H]⁺

IR (ATR) [cm⁻¹] 3080, 3007, 2945, 2847, 1439, 1403, 1287, 1128, 1070, 799, 603

¹H-NMR (400 MHz, DMSO-d6) δ 8.40 (s, 1H), 8.13 (d, J=8.0 Hz, 1H), 7.76 (d, J=8.0 Hz, 1H), 5.06 (s, 2H), 3.90 (d, J=8.1 Hz, 6H).

¹³C-NMR (100 MHz, DMSO-d6) δ 165.64, 164.97, 143.59, 132.86, 132.48, 131.19, 129.78, 129.38, 52.53 (d, J=10.1 Hz), 30.79.

### 4-((3-Chlorophenoxy) methyl)isophthalic acid, dimethyl ester (5)

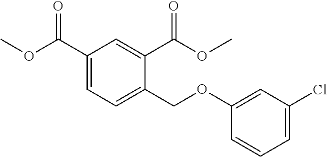 [SMILES: COC(=O)c1ccc(COc2cccc(Cl)c2)c(C(=O)OC)c1]

Potassium carbonate (1.64 g; 11.88 mmol) was weighed into a 100-mL round bottom flask. This was suspended in acetone (50 mL) and to this was added, 3-chlorophenol (1.53 g; 11.88 mmol). This was heated to 50° C., and at this point, compound 4 (3.10 g; 10.80 mmol) in acetone was added. The reaction was stirred for 6 h at 70° C. (reflux). After reaction completion, the solvent was removed in vacuo and the residue was taken up in EtOAc. The organic phase was washed with a 10% aq. NaOH solution, dried over Na₂SO₄, and evaporated under reduced pressure to dryness. The crude oil obtained was purified by flash chromatography (SiO₂, PE/EtOAc 4:1) to produce the final product, 5 (2.87 g; 79.4% yield); mp 111.6° C.

MS (FABₚₒₛ) (m/z): 335.1 [M+H]⁺

IR (ATR) [cm⁻¹] 3072, 2994, 2949, 2839, 1715, 1590, 1433, 1300, 1231, 1085, 879, 751, 677

¹H-NMR (400 MHz, DMSO-d6) δ 8.46 (s, 1H), 8.18 (d, J=8.1 Hz, 1H), 7.82 (d, J=7.7 Hz, 1H), 7.32 (d, J=8.0 Hz, 1H), 7.14-6.91 (m, 3H), 5.52 (s, 2H), 3.88 (d, J=4.4 Hz, 6H).

¹³C-NMR (100 MHz, DMSO-d6) δ 166.24, 165.55, 159.29, 143.47, 134.15, 133.18, 131.28 (d, J=10.4 Hz), 129.45, 128.86 (d, J=17.0 Hz), 121.43, 115.22, 114.12, 67.96, 52.81.

### 4-((3-Chlorophenoxy)methyl)isophthalic acid (6)

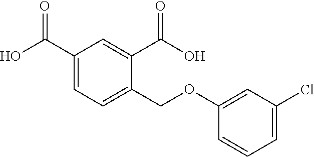 [SMILES: O=C(O)c1ccc(COc2cccc(Cl)c2)c(C(=O)O)c1]

Compound 6 was synthesized following the general method B. Starting material: compound 5 (2.00 g; 5.97 mmol); Purification: The solvent was removed in vacuo and the residue taken up in H₂O. The aqueous phase was acidified with 10% aq. HCl producing a white precipitate 6 that was collected by filtration (1.81 g; 98.8% yield); mp 243.4° C.

MS (FAB_{neg}) (m/z): 306.9 [M−H]⁻

IR (ATR) [cm⁻¹] 2908, 2851, 2639, 2524, 1681, 1596, 1431, 1249, 869, 752, 676, 493

¹H-NMR (400 MHz, DMSO-d6) δ 13.25 (s, 2H), 8.47 (s, 1H), 8.13 (d, J=8.0 Hz, 1H), 7.77 (d, J=7.9 Hz, 1H), 7.38-7.24 (m, 1H), 7.06 (d, J=12.5 Hz, 1H), 7.01 (t, J=7.4 Hz, 1H), 6.96 (d, J=8.3 Hz, 1H), 5.53 (s, 2H).

¹³C-NMR (100 MHz, DMSO-d6) δ 167.26, 166.32, 159.06, 142.63, 133.75, 132.60, 131.29, 130.98, 130.20, 129.55, 128.04, 120.95, 114.92, 113.70, 67.78.

### 3-Chloro-11-oxo-6,11-dihydrobenzo[b,e]oxepino-9-carboxylic acid (7)

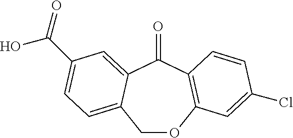 [SMILES: O=C(O)c1ccc2c(c1)C(=O)c1ccc(Cl)cc1OC2]

Compound 7 was synthesized using general method D, variant A. Starting material: compound 6 (0.40 g; 1.30 mmol); Purification: white solid 7 (0.21 g; 55.8% yield); mp 245.6° C.

MS (FAB_{neg}) (m/z): 288.8 [M−H]⁻

IR (ATR) [cm⁻¹] 2984, 2814, 2533, 2361, 1681, 1596, 1486, 1414, 1251, 1040, 869, 829, 753, 599, 678

¹H-NMR (400 MHz, DMSO-d6) δ 13.71-12.82 (s, 1H), 8.31 (s, 1H), 8.13 (d, J=9.1 Hz, 2H), 7.72 (d, J=7.8 Hz, 1H), 7.36-7.15 (m, 2H), 5.42 (s, 2H).

¹³C-NMR (100 MHz, DMSO-d6) δ 167.28, 166.33, 159.10, 142.63, 133.76, 132.60, 131.28, 130.99, 130.22, 129.60, 128.09, 120.97, 114.95, 113.73, 67.80.

### 3-Chloro-11-oxo-6,11-dihydrobenzo[b,e]oxepin-9-carboxylmethylester (8a)

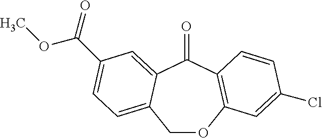 [SMILES: COC(=O)c1ccc2c(c1)C(=O)c1ccc(Cl)cc1OC2]

Compound 8a was synthesized using the general method A, variant A. Starting material: compound 7 (2.00 g; 6.93 mmol); Workup: After completion, the excess alcohol was removed in vacuo. The residue was taken up in EtOAc. The organics were washed several times with 20% aq. NaOH, dried over Na₂SO₄, and removed under reduced pressure to dryness to obtain the crude product as an off-white solid (2.01 g; 95.8% yield); mp 271.4° C.

MS (FABₚₒₛ) (m/z): 303.1 [M+H]⁺

IR (ATR) [cm⁻¹] 2982, 2962, 2925, 1639, 1594, 1567, 1376, 1263, 1196, 1017, 761, 644

¹H-NMR (400 MHz, DMSO-d6) δ 8.33 (s, 1H), 8.22 (d, J=7.8 Hz, 2H), 8.18-8.08 (m, 2H), 7.77 (d, J=7.8 Hz, 1H), 7.29 (d, J=7.1 Hz, 2H), 5.44 (s, 3H).

### 3-((2,4-Difluorophenyl)amino)-11-oxo-6,11-dihydrobenzo[b,e]oxepin-9-carboxylmethylester (15a)

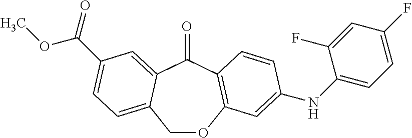 [SMILES: COC(=O)c1ccc2c(c1)C(=O)c1ccc(Nc3ccc(F)cc3F)cc1OC2]

Compound 15a was synthesized using general method E, variant A. Starting material: Compound 8a (0.100 g; 0.32 mmol), 2,4-Difluoro aniline (Ile) (0.063 g; 0.49 mmol); Workup: flash chromatography (SiO₂, PE/EtOAc 4:1) to obtain a yellow solid (0.102 g; 63.1% yield), mp 162.7° C.

MS (HRMS-EI) (m/z): 396.1038 (berechnet: 396.1042) [M+H]⁺

IR (ATR) [cm⁻¹] 3358, 3334, 1709, 1607, 1566, 1506, 1259, 1139, 840, 762, 526

¹H-NMR (400 MHz, DMSO-d6) δ 8.81 (s, 1H), 8.37 (s, 1H), 8.15 (d, J=7.5 Hz, 1H), 8.03 (dd, J=8.9, 2.2 Hz, 1H), 7.74-7.65 (m, 1H), 7.48-7.31 (m, 2H), 7.10 (dd, J=22.3, 14.5 Hz, 1H), 6.65 (d, J=8.9 Hz, 1H), 6.26 (s, 1H), 5.28 (s, 2H), 3.89 (d, J=1.8 Hz, 3H).

¹³C-NMR (100 MHz, DMSO-d6) δ 185.96, 165.41, 162.87, 152.18, 140.64, 140.05, 133.51, 132.56, 130.16, 129.82, 128.79, 126.84 (d, J=6.8 Hz), 124.16, 116.45, 112.05, 111.83, 109.87, 105.76, 104.76, 104.76, 101.73, 72.46, 52.39.

### 3-Chlor-N-methyl-11-oxo-6,11-dihydrobenzo[b,e]oxepin-9-carbonsäureamid (9)

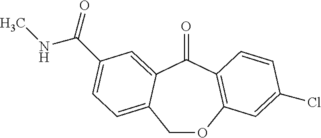 [SMILES: CNC(=O)c1ccc2c(c1)C(=O)c1ccc(Cl)cc1OC2]

0.27 g (0.94 mmol) of 3-chloro-11-oxo-6,11-dihydrobenzo[b,e]oxepino-9-carboxylic acid (7) were dissolved in 15 mL of dry THF under an atmosphere of argon and 0.38 mL (5.14 mmol) SOCl₂ were added. After heating to reflux for 1 h all volatiles were removed in vacuo. The residue was re-dissolved with 15 ml of dry THF and at RT 5 ml of a 2.5M solution of methylamine in THF were added. The mixture was left stirring overnight and partitioned between water and ethyl acetate. The aqueous phase was extracted twice with ethyl acetate and the combined organic phases were concentrated i.v. Chromatography (PE/EtOAc) over silica gel yielded the target compound as a yellow solid.

C₁₆H₁₂ClNO₃ (M=301.72 g/mol)

Yield 0,202 g (70.9%)

Melting point 80.1° C.

Masse (FABₚₒₛ) (m/z): 302.1 [M+H]⁺

¹H-NMR (400 MHz, DMSO-d6) δ 8.69 (d, J=3.6 Hz, 1H), 8.22 (d, J=25.4 Hz, 1H), 8.10 (dd, J=17.2, 10.4 Hz, 2H), 7.72 (dd, J=28.1, 7.3 Hz, 1H), 7.27 (d, J=7.6 Hz, 2H), 5.43 (d, J=25.3 Hz, 2H), 2.79 (d, J=4.2 Hz, 3H).

¹³C-NMR (100 MHz, DMSO-d6) δ 188.61, 165.33, 161.24, 139.84, 139.42, 138.05, 135.31, 133.12, 131.64, 128.64, 127.71, 123.84, 122.70, 120.30, 72.61, 57.96.

### 3-Chlor-9-(morpholin-4-carbonyl)dibenzo[b,e]oxepin-11(6H)-on (10)

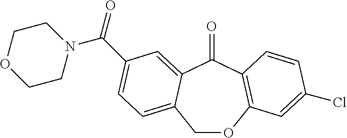 [SMILES: O=C1c2cc(C(=O)N3CCOCC3)ccc2COc2cc(Cl)ccc21]

0.20 g (0.69 mmol) 3-Chloro-11-oxo-6,11-dihydrobenzo[b,e]oxepino-9-carboxylic acid (7) were dissolved with dichloromethane. 0.25 mL (3.46 mmol) thionyl chloride were added dropwise, the mixture was then heated to reflux for 1 h. The mixture is then added dropwise to a cooled (ice) solution of 2 mL (22.50 mmol) morpholin in 5 mL of dichloromethane. After stirring at RT for 2 h, all volatiles were removed i.v. The residue was dissolved with ethyl acetate and extracted 3× with dil. aq. NaOH. After evaporation, the target compound remains as a brownish solid.

C₁₉H₁₆ClNO₄ (M=357.79 g/mol)

Yield 0.24 g (96.8%)

Melting point 184.5° C.

Masse (FABₚₒₛ) (m/z): 358.0 [M+H]⁺

IR (ATR) [cm⁻¹] 3068, 2949, 2912, 2851, 2352, 1628, 1272, 1110, 1012, 753, 577

¹H-NMR (400 MHz, DMSO-d6) δ 8.11 (d, J=9.2 Hz, 1H), 7.75 (d, J=13.8 Hz, 1H), 7.69 (dd, J=17.2, 7.7 Hz, 2H), 7.28 (dd, J=3.3, 1.5 Hz, 2H), 5.39 (s, 2H), 3.58 (d, J=39.8 Hz, 7H).

¹³C-NMR (100 MHz, DMSO-d6) δ 188.82, 137.01 (d, J=11.3 Hz), 133.57, 127.95, 124.15, 123.04, 120.68, 73.00, 66.34.

### 3-Chlorobenzyliodide (50)

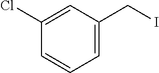

4.1 g (25 mmol) of 3-chlorobenzylchloride were dissolved with 18 ml of acetone. 6.5 g of sodium iodide were added. The mixture was heated to reflux for 2 h. The mixture was cooled to RT and partitioned between water and ethyl acetate. The organic phase was dried with sodium sulfate and concentrated to obtain 6.15 g (96%) of a brownish viscous oil of a purity sufficient for the following step.

### 4-(3-chlorophenylethyl)isophthalic acid dimethylester (51)

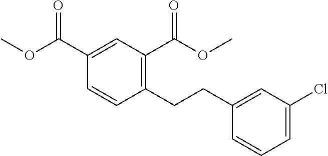 [SMILES: COC(=O)c1ccc(CCc2cccc(Cl)c2)c(C(=O)OC)c1]

18 ml of a 2.5 M solution of n-BuLi in hexane were added with ice cooling to a solution of 6.35 ml of diisopropylamine in THF. After 2 h of stirring, the mixture was cooled to −78° C. and a solution of 5.0 g (24 mmol) of 4-methylisophthalic acid dimethylester in 5 ml of THF was added dropwise over 15 min. After 1 h of stirring at −78° C., 6.3 g (25 mmol) of (50) was added at the same temperature dropwise over 30 min. The mixture was stirred for another h at −78° C. and then allowed to reach RT. The mixture was then partitioned between saturated aq. NH₄Cl solution and ethyl acetate. The organic phase was dried over sodium sulfate and concentrated to dryness i.v. Purification by chromatography with PE/EtOAc over silica gel yielded 3.92 g (50%) of the target compound as a white solid.

Masse (ESIₚₒₛ) (m/z): 333.1 [M+H]⁺

¹H-NMR (400 MHz, DMSO-d6) δ 8.36 (s, 1H), 8.03 (d, J=8.0 Hz, 1H), 7.51 (d, J=8.0 Hz, 1H), 7.32-7.22 (m, 3H), 7.15 (d, J=7.2 Hz, 1H), 3.86 (s, 6H), 3.25-3.17 (m, 2H), 2.87-2.77 (m, 2H).

¹³C-NMR (100 MHz, DMSO-d6) δ 166.31, 165.30, 147.83, 143.64, 132.86, 132.23, 131.77, 130.91, 130.03, 129.54, 128.15, 127.75, 127.03, 125.92, 52.26, 52.20, 36.38, 35.32.

### 4-(3-chlorophenethyl)isophthtalic acid (52)

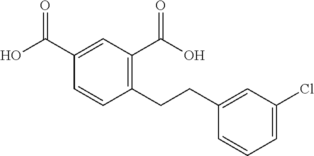 [SMILES: O=C(O)c1ccc(CCc2cccc(Cl)c2)c(C(=O)O)c1]

the title compound was prepared from (51) according to general method B with 92% yield.

Masse (FAB_{neg}) (m/z): 303.4 [M−H]⁻

¹H-NMR (400 MHz, DMSO-d6) δ 13.17 (s, 2H), 8.41 (s, 1H), 7.99 (d, J=8.0 Hz, 1H), 7.44 (d, J=8.0 Hz, 1H), 7.34-7.15 (m, 4H), 3.30-3.22 (m, 2H), 2.90-2.81 (m, 2H).

¹³C-NMR (100 MHz, DMSO-d6) δ 167.93, 166.54, 147.36, 143.99, 132.91, 132.10, 131.44, 131.40, 130.51, 130.05, 128.89, 128.16, 126.99, 125.90, 36.49, 35.60.

### 8-Chloro-5-oxo-10,11-dihydro-5H-dibenzo[a,d]cyclohepten-3-carboxylic acid (53)

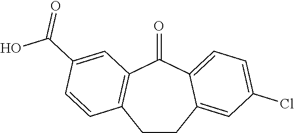 [SMILES: O=C(O)c1ccc2c(c1)C(=O)c1ccc(Cl)cc1CC2]

The title compound was prepared from (52) according to general method D, variant A in 86% yield.

Masse (ESI_{neg}) (m/z): 285.0 [M−H]⁻

¹H-NMR (400 MHz, DMSO-d6) δ 13.66 (s, 1H), 8.44 (s, 2H), 8.06-7.99 (m, 2H), 7.93-7.86 (m, 2H), 7.50-7.38 (m, 6H), 3.27-3.12 (m, 8H).

¹³C-NMR (100 MHz, DMSO-d6) δ 192.44, 166.48, 146.84, 144.31, 137.57, 137.45, 136.28, 132.98, 132.33, 131.36, 130.34, 129.31, 129.21, 126.77, 33.89, 33.41.

| | | Starting | | | | Yield |
|---|---|---|---|---|---|---|
| Entry | Name | Material | Starting material | Method | Variant | [%] |
| 8b | Ethyl-3-chloro-11-oxo-6,11- | 7 | ethanol | A | A | 98 |
| | dihydrobenzo[b,e]oxepino- | | | | | |
| | carboxylate | | | | | |
| 8c | Propyl-3-chloro-11-oxo-6,11- | 7 | n-propanol | A | A | 70 |
| | dihydrobenzo[b,e]oxepino- | | | | | |
| | carboxylate | | | | | |
| 15b | ethyl 3-((2,4- | 8b | 2,4-difluoroanilin | E | A | 82 |
| | difluorophenyl)amino)-11-oxo-6,11- | | | | | |
| | dihydrodibenzo[b,e]oxepine- | | | | | |
| | 9-carboxylate | | | | | |
| 15c | propyl 3-((2,4- | 8c | 2,4-difluoroanilin | E | A | 39 |
| | difluorophenyl)amino)-11-oxo-6,11- | | | | | |
| | dihydrodibenzo[b,e]oxepine- | | | | | |
| | 9-carboxylate | | | | | |
| 16 | 3-((2,4-difluorophenyl)amino)- | 9 | 2,4-difluoroanilin | E | A | 34 |
| | N-methyl-11-oxo-6,11- | | | | | |
| | dihydrodibenzo[b,e]oxepine- | | | | | |
| | 9-carboxamide | | | | | |
| 17 | 3-((2,4-difluorophenyl)amino)- | 10 | 2,4-difluoroanilin | E | A | 66 |
| | 9-(morpholine-4- | | | | | |
| | carbonyl)dibenzo[b,e]oxepin- | | | | | |
| | 11(6H)-one | | | | | |
| 29 | 3-((2,4-difluorophenyl)amino)- | 15a | — | B | — | 92 |
| | 11-oxo-6,11- | | | | | |
| | dihydrodibenzo[b,e]oxepine- | | | | | |
| | 9-carboxylic acid | | | | | |
| 30 | 2-hydroxyethyl 3- | 29 | ethylenglycol | A | C | 31 |
| | ((2,4-difluorophenyl)amino)- | | | | | |
| | 11-oxo-6,11- | | | | | |
| | dihydrodibenzo[b,e]oxepine- | | | | | |
| | 9-carboxylate | | | | | |
| 31 | 2-morpholinoethyl | 29 | 2- | A | C | 61 |
| | 3-((2,4-difluorophenyl)amino)- | | morpholinoethanol | | | |
| | 11-oxo-6,11- | | | | | |
| | dihydrodibenzo[b,e]oxepine- | | | | | |
| | 9-carboxylate | | | | | |
| 32 | 3-((2,4-difluorophenyl)amino)- | 29 | ammonia (conc. | C | B | 62 |
| | 11-oxo-6,11- | | aq. solution) | | | |
| | dihydrodibenzo[b,e]oxepine- | | | | | |
| | 9-carboxamide | | | | | |
| 33 | 3-((2,4-difluorophenyl)amino)- | 29 | ethanolamin | D | B | 34 |
| | N-(2-hydroxyethyl)-11-oxo-6,11- | | | | | |
| | dihydrodibenzo[b,e]oxepine- | | | | | |
| | 9-carboxamide | | | | | |
| 34 | 3-((2,4-difluorophenyl)amino)- | 29 | 2-amino-1,3- | C | B | 67 |
| | N-(1,3-dihydroxypropan-2-yl)- | | propandiol | | | |
| | 11-oxo-6,11- | | | | | |
| | dihydrodibenzo[b,e]oxepine- | | | | | |
| | 9-carboxamide | | | | | |
| 35 | 3-((2,4-difluorophenyl)amino)- | 29 | TRIS ((tris- | C | B | 70 |
| | N-(1,3-dihydroxy-2- | | hydroxymethyl)- | | | |
| | (hydroxymethyl)propan-2-yl)- | | aminomethane) | | | |
| | 11-oxo-6,11- | | | | | |
| | dihydrodibenzo[b,e]oxepine- | | | | | |
| | 9-carboxamide | | | | | |
| 36 | 3-((2,4-difluorophenyl)amino)- | 29 | 1-amino-2-butanol | C | B | 76 |
| | N-(2-hydroxybutyl)-11-oxo-6,11- | | | | | |
| | dihydrodibenzo[b,e]oxepine- | | | | | |
| | 9-carboxamide | | | | | |
| 37 | 3-((2,4-difluorophenyl)amino)- | 29 | propanolamine | C | B | 70 |
| | N-(3-hydroxypropyl)-11-oxo-6,11- | | | | | |
| | dihydrodibenzo[b,e]oxepine- | | | | | |
| | 9-carboxamide | | | | | |
| 38 | (R)-3-((2,4-difluorophenyl)amino)- | 29 | (R)-1-amino-2,3- | C | B | 36 |
| | N-(2,3-dihydroxypropyl)-11-oxo-6,11- | | propandiol | | | |
| | dihydrodibenzo[b,e]oxepine- | | | | | |
| | 9-carboxamide | | | | | |
| 39 | (S)-3-((2,4-difluorophenyl)amino)- | 29 | (S)-1-amino-2,3- | C | B | 45 |
| | N-(2,3-dihydroxypropyl)-11-oxo-6,11- | | propandiol | | | |
| | dihydrodibenzo[b,e]oxepine- | | | | | |
| | 9-carboxamide | | | | | |
| 40 | 3-((2,4-difluorophenyl)amino)- | 29 | 1-aminopropan-2- | C | B | 61 |
| | N-(2-hydroxypropyl)-11-oxo-6,11- | | ol | | | |
| | dihydrodibenzo[b,e]oxepine- | | | | | |
| | 9-carboxamide | | | | | |
| 41 | 3-((2,4-difluorophenyl)amino)- | 29 | 3-amino-1,5- | C | B | 43 |
| | N-(1,5-dihydroxypentan-3-yl)- | | pentandiol | | | |
| | 11-oxo-6,11- | | | | | |
| | dihydrodibenzo[b,e]oxepine- | | | | | |
| | 9-carboxamide | | | | | |
| 42 | 3-((2,4-difluorophenyl)amino)- | 29 | 2- | C | B | 53 |
| | N-(2-morpholinoethyl)-11-oxo-6,11- | | morpholinoethylamine | | | |
| | dihydrodibenzo[b,e]oxepine- | | | | | |
| | 9-carboxamide | | | | | |
| 43 | 3-((2,4-difluorophenyl)amino)- | 29 | 2-(4- | C | B | 56 |
| | N-(2-(4-methylpiperazin-1- | | methylpiperazin-1- | | | |
| | yl)ethyl)-11-oxo-6,11- | | yl)ethylamine | | | |
| | dihydrodibenzo[b,e]oxepine- | | | | | |
| | 9-carboxamide | | | | | |
| 54 | Methyl-8-chloro-5-oxo-10,11- | 53 | methanol | A | A | 99 |
| | dihydro-5H- | | | | | |
| | dibenzo[a,d]cyclohepten- | | | | | |
| | 3-carboxylate | | | | | |
| 55 | methyl 8-((2,4- | 54 | 2,4-difluoroanilin | E | A | 89 |
| | difluorophenyl)amino)-5- | | | | | |
| | oxo-10,11-dihydro-5H- | | | | | |
| | dibenzo[a,d][7]annulene- | | | | | |
| | 3-carboxylate | | | | | |
| 56 | 8-((2,4-difluorophenyl)amino)- | 55 | — | B | — | 99 |
| | 5-oxo-10,11-dihydro-5H- | | | | | |
| | dibenzo[a,d][7]annulene- | | | | | |
| | 3-carboxylic acid | | | | | |
| 58 | methyl 8-((2-aminophenyl)amino)- | 54 | 1,2- | E | B | 95 |
| | 5-oxo-10,11-dihydro-5H- | | diaminobenzene | | | |
| | dibenzo[a,d][7]annulene- | | | | | |
| | 3-carboxylate | | | | | |
| 60 | 8-((2-aminophenyl)amino)- | 58 | — | B | — | 96 |
| | 5-oxo-10,11-dihydro-5H- | | | | | |
| | dibenzo[a,d][7]annulene- | | | | | |
| | 3-carboxylic acid | | | | | |
| 61 | methyl 8-((2-nitrophenyl)amino)- | 54 | 2-nitroanilin | E | A | 80 |
| | 5-oxo-10,11-dihydro-5H- | | | | | |
| | dibenzo[a,d][7]annulene- | | | | | |
| | 3-carboxylate | | | | | |
| 62 | 8-((2-nitrophenyl)amino)- | 61 | — | B | — | 95 |
| | 5-oxo-10,11-dihydro-5H- | | | | | |
| | dibenzo[a,d][7]annulene- | | | | | |
| | 3-carboxylic acid | | | | | |
| 65a | 8-((2,4-difluorophenyl)amino)- | 56 | dimethylamin | C | C | 77 |
| | N,N-dimethyl-5-oxo-10,11- | | hydrochloride | | | |
| | dihydro-5H- | | | | | |
| | dibenzo[a,d][7]annulene- | | | | | |
| | 3-carboxamide | | | | | |
| 66 | 8-((2,4-difluorophenyl)amino)- | 56 | 2- | C | C | 45 |
| | 5-oxo-N-phenethyl-10,11- | | phenylethylamine | | | |
| | dihydro-5H- | | | | | |
| | dibenzo[a,d][7]annulene- | | | | | |
| | 3-carboxamide | | | | | |
| 67 | 8-((2,4-difluorophenyl)amino)- | 56 | N-methyl-2- | C | C | 55 |
| | N-methyl-5-oxo-N-phenethyl- | | phenylethylamine | | | |
| | 10,11-dihydro-5H- | | | | | |
| | dibenzo[a,d][7]annulene- | | | | | |
| | 3-carboxamide | | | | | |
| 68 | 8-((2,4-difluorophenyl)amino)- | 56 | 2-(4- | C | C | 39 |
| | N-(4-fluorophenethyl)-5-oxo- | | fluorophenyl)ethylamine | | | |
| | 10,11-dihydro-5H- | | | | | |
| | dibenzo[a,d][7]annulene- | | | | | |
| | 3-carboxamide | | | | | |
| 69 | 8-((2,4-difluorophenyl)amino)- | 56 | 2-(4- | C | C | 28 |
| | N-(4-hydroxyphenethyl)- | | hydroxyphenyl)ethylamine | | | |
| | 5-oxo-10,11-dihydro-5H- | | | | | |
| | dibenzo[a,d][7]annulene- | | | | | |
| | 3-carboxamide | | | | | |
| 70 | N-(2-(1H-imidazol-5-yl)ethyl)- | 56 | histamin | C | C | 29 |
| | 8-((2,4-difluorophenyl)amino)- | | dihydrochloride | | | |
| | 5-oxo-10,11-dihydro-5H- | | | | | |
| | dibenzo[a,d][7]annulene- | | | | | |
| | 3-carboxamide | | | | | |
| 71 | 8-((2,4-difluorophenyl)amino)- | 56 | 2-(thiophen-2- | C | C | 53 |
| | 5-oxo-N-(2-(thiophen-2- | | yl)ethylamine | | | |
| | yl)ethyl)-10,11-dihydro-5H- | | | | | |
| | dibenzo[a,d][7]annulene- | | | | | |
| | 3-carboxamide | | | | | |
| 72 | N-(2-(1H-indol-3-yl)ethyl)- | 56 | Tryptamine | C | C | 81 |
| | 8-((2,4-difluorophenyl)amino)- | | hydrochloride | | | |
| | 5-oxo-10,11-dihydro-5H- | | | | | |
| | dibenzo[a,d][7]annulene- | | | | | |
| | 3-carboxamide | | | | | |
| 73 | 8-((2,4-difluorophenyl)amino)- | 56 | 3- | C | C | 23 |
| | 5-oxo-N-(3-phenylpropyl)- | | phenylpropylamine | | | |
| | 10,11-dihydro-5H- | | | | | |
| | dibenzo[a,d][7]annulene- | | | | | |
| | 3-carboxamide | | | | | |
| 74 | N-(2-(2-oxa-6- | 56 | 2-(2-oxa-6- | C | C | 79 |
| | azaspiro[3.3]heptan-6-yl)ethyl)- | | azaspiro[3.3]heptan-6- | | | |
| | 8-((2,4-difluorophenyl)amino)- | | yl)ethylamine | | | |
| | 5-oxo-10,11-dihydro-5H- | | | | | |
| | dibenzo[a,d][7]annulene- | | | | | |
| | 3-carboxamide | | | | | |
| 75 | 8-((2,4-difluorophenyl)amino)- | 56 | 1-phenyl-1- | C | C | 44 |
| | 5-oxo-N-(1-phenylcyclopropyl)- | | cyclopropylamine | | | |
| | 10,11-dihydro-5H- | | | | | |
| | dibenzo[a,d][7]annulene- | | | | | |
| | 3-carboxamide | | | | | |
| 76 | N-(1-cyclohexylethyl)-8- | 56 | 1- | C | C | 31 |
| | ((2,4-difluorophenyl)amino)- | | cyclohexylethylamine | | | |
| | 5-oxo-10,11-dihydro-5H- | | | | | |
| | dibenzo[a,d][7]annulene- | | | | | |
| | 3-carboxamide | | | | | |
| 77 | 8-((2,4-difluorophenyl)amino)- | 56 | hydroxylamine | C | C | 77 |
| | N-hydroxy-5-oxo-10,11-dihydro-5H- | | hydrochloride | | | |
| | dibenzo[a,d][7]annulene- | | | | | |
| | 3-carboxamide | | | | | |
| 78 | 8-((2,4-difluorophenyl)amino)- | 56 | TRIS hydrochloride | C | C | 23 |
| | N-(1,3-dihydroxy-2- | | | | | |
| | (hydroxymethyl)propan-2-yl)-5-oxo- | | | | | |
| | 10,11-dihydro-5H- | | | | | |
| | dibenzo[a,d][7]annulene- | | | | | |
| | 3-carboxamide | | | | | |
| 79 | 8-((2,4-difluorophenyl)amino)- | 56 | D-glucosamine | C | C | 56 |
| | 5-oxo-N-((2R,3R,4R,5S,6R)-2,4,5- | | hydrochloride | | | |
| | trihydroxy-6-(hydroxymethyl)tetrahydro- | | | | | |
| | 2H-pyran-3-yl)-10,11-dihydro-5H- | | | | | |
| | dibenzo[a,d][7]annulene- | | | | | |
| | 3-carboxamide | | | | | |
| 80 | 8-((2,4-difluorophenyl)amino)- | 56 | 2(2- | C | C | 35 |
| | N-(2-(2-hydroxyethoxy)ethyl)- | | aminoethoxy)ethanol | | | |
| | 5-oxo-10,11-dihydro-5H- | | | | | |
| | dibenzo[a,d][7]annulene- | | | | | |
| | 3-carboxamide | | | | | |
| 81 | 8-((2,4-difluorophenyl)amino)- | 56 | 2-((2- | C | C | 51 |
| | N-(2-((2-hydroxyethyl)amino)ethyl)- | | aminoethyl)amino)ethanol | | | |
| | 5-oxo-10,11-dihydro-5H- | | | | | |
| | dibenzo[a,d][7]annulene- | | | | | |
| | 3-carboxamide | | | | | |
| 82 | N-(2-aminoethyl)-8- | 56 | ethylen-1,2- | C | C | 61 |
| | ((2,4-difluorophenyl)amino)- | | diamine | | | |
| | 5-oxo-10,11-dihydro-5H- | | | | | |
| | dibenzo[a,d][7]annulene- | | | | | |
| | 3-carboxamide | | | | | |
| 83 | N-(3-amino-3-oxopropyl)-8-((2,4- | 56 | 3-aminopropionic | C | C | 82 |
| | difluorophenyl)amino)-5-oxo-10,11- | | acid amide | | | |
| | dihydro-5H- | | | | | |
| | dibenzo[a,d][7]annulene- | | | | | |
| | 3-carboxamide | | | | | |
| 84 | 8-((2,4-difluorophenyl)amino)- | 56 | 2,2,2- | C | C | 63 |
| | 5-oxo-N-(2,2,2-trifluoroethyl)- | | trifluoroethylamine | | | |
| | 10,11-dihydro-5H- | | | | | |
| | dibenzo[a,d][7]annulene- | | | | | |
| | 3-carboxamide | | | | | |
| 85 | 8-((2,4-difluorophenyl)amino)- | 56 | n-octylamine | C | Cf | 25 |
| | N-octyl-5-oxo-10,11-dihydro-5H- | | | | | |
| | dibenzo[a,d][7]annulene- | | | | | |
| | 3-carboxamide | | | | | |
| 86 | 8-((2,4-difluorophenyl)amino)- | 56 | n-hexadecylamine | C | C | 46 |
| | N-hexadecyl-5-oxo-10,11-dihydro-5H- | | | | | |
| | dibenzo[a,d][7]annulene- | | | | | |
| | 3-carboxamide | | | | | |
| 87a | tert-butyl 4-(2-(8-((2,4- | 56 | 4-((2-aminoethyl)piperazin | C | C | 49 |
| | difluorophenyl)amino)- | | carboxylic acid | | | |
| | 5-oxo-10,11-dihydro-5H- | | tert-butylester | | | |
| | dibenzo[a,d][7]annulene- | | | | | |
| | 3-carboxamido)ethyl)piperazine-1- | | | | | |
| | carboxylate | | | | | |
| 87b | 8-((2,4-difluorophenyl)amino)- | 87a | — | I | A | 95 |
| | 5-oxo-N-(2-(piperazin-1-yl)ethyl)- | | | | | |
| | 10,11-dihydro-5H- | | | | | |
| | dibenzo[a,d][7]annulene- | | | | | |
| | 3-carboxamide | | | | | |
| 88 | 2-((2,4-difluorophenyl)amino)- | 56 | piperidin | C | C | 40 |
| | 7-(piperidine-1-carbonyl)- | | | | | |
| | 10,11-dihydro-5H- | | | | | |
| | dibenzo[a,d][7]annulen-5-one | | | | | |
| 89 | 8-((2,4-difluorophenyl)amino)- | 56 | anilin | C | Cf | 97 |
| | 5-oxo-N-phenyl-10,11-dihydro-5H- | | | | | |
| | dibenzo[a,d][7]annulene- | | | | | |
| | 3-carboxamide | | | | | |
| 90a | (S)-2-((tert-butoxycarbonyl)amino)- | 56 | N-Boc-serin | C | C | 43 |
| | 3-methoxy-3-oxopropyl 8-((2,4- | | methyl ester (Boc- | | | |
| | difluorophenyl)amino)- | | ser-OMe) | | | |
| | 5-oxo-10,11-dihydro-5H- | | | | | |
| | dibenzo[a,d][7]annulene- | | | | | |
| | 3-carboxylate | | | | | |
| 90b | (S)-2-amino-3-methoxy-3-oxopropyl | 90a | — | I | A | 65 |
| | 8-((2,4-difluorophenyl)amino)- | | | | | |
| | 5-oxo-10,11-dihydro-5H- | | | | | |
| | dibenzo[a,d][7]annulene- | | | | | |
| | 3-carboxylate | | | | | |
| 91a | methyl (8-((2,4-difluorophenyl)amino)- | 56 | glycin methyl ester | C | C | 58 |
| | 5-oxo-10,11-dihydro-5H- | | hydrochloride | | | |
| | dibenzo[a,d][7]annulene- | | | | | |
| | 3-carbonyl)glycinate | | | | | |
| 91b | (8-((2,4-difluorophenyl)amino)- | 91a | — | B | — | 62 |
| | 5-oxo-10,11-dihydro-5H- | | | | | |
| | dibenzo[a,d][7]annulene- | | | | | |
| | 3-carbonyl)glycine | | | | | |
| 92 | methyl (8-((2,4-difluorophenyl)amino)- | 56 | L-alanin methyl | C | C | 38 |
| | 5-oxo-10,11-dihydro-5H- | | ester hydrochloride | | | |
| | dibenzo[a,d][7]annulene- | | | | | |
| | 3-carbonyl)-L-alaninate | | | | | |
| 93a | methyl 3-(8-((2,4-difluorophenyl)amino)- | 56 | β-alanin methyl | C | C | 95 |
| | 5-oxo-10,11-dihydro-5H- | | ester hydrochloride | | | |
| | dibenzo[a,d][7]annulene- | | | | | |
| | 3-carboxamido)propanoate | | | | | |
| 93b | 3-(8-((2,4-difluorophenyl)amino)- | 93a | — | B | — | 68 |
| | 5-oxo-10,11-dihydro-5H- | | | | | |
| | dibenzo[a,d][7]annulene- | | | | | |
| | 3-carboxamido)propanoic acid | | | | | |
| 94a | methyl 4-(8-((2,4-difluorophenyl)amino)- | 56 | 4-aminobutyric | C | C | 86 |
| | 5-oxo-10,11-dihydro-5H- | | acid methyl ester | | | |
| | dibenzo[a,d][7]annulene- | | hydrochloride | | | |
| | 3-carboxamido)butanoate | | | | | |
| 94b | 4-(8-((2,4-difluorophenyl)amino)- | 94a | — | B | — | 41 |
| | 5-oxo-10,11-dihydro-5H- | | | | | |
| | dibenzo[a,d][7]annulene- | | | | | |
| | 3-carboxamido)butanoic acid | | | | | |
| 95a | methyl (8-((2,4-difluorophenyl)amino)- | 56 | L-phenylalanin | C | C | 90 |
| | 5-oxo-10,11-dihydro-5H- | | methyl ester | | | |
| | dibenzo[a,d][7]annulene- | | hydrochloride | | | |
| | 3-carbonyl)-L-phenylalaninate | | | | | |
| 95b | (8-((2,4-difluorophenyl)amino)- | 95a | — | B | — | 82 |
| | 5-oxo-10,11-dihydro-5H- | | | | | |
| | dibenzo[a,d][7]annulene- | | | | | |
| | 3-carbonyl)-L-phenylalanine | | | | | |
| 96a | dimethyl (8-((2,4-difluorophenyl)amino)- | 56 | L-glutamic acid | C | C | 97 |
| | 5-oxo-10,11-dihydro-5H- | | dimethyl ester | | | |
| | dibenzo[a,d][7]annulene- | | hydrochloride | | | |
| | 3-carbonyl)-L-glutamate | | | | | |
| 96b | (8-((2,4-difluorophenyl)amino)- | 96a | — | B | — | 66 |
| | 5-oxo-10,11-dihydro-5H- | | | | | |
| | dibenzo[a,d][7]annulene- | | | | | |
| | 3-carbonyl)-L-glutamic acid | | | | | |
| 97a | methyl (8-((2,4-difluorophenyl)amino)- | 56 | L-methionin | C | C | 39 |
| | 5-oxo-10,11-dihydro-5H- | | methyl ester | | | |
| | dibenzo[a,d][7]annulene- | | hydrochloride | | | |
| | 3-carbonyl)-L-methioninate | | | | | |
| 97b | (8-((2,4-difluorophenyl)amino)- | 97a | — | B | — | 71 |
| | 5-oxo-10,11-dihydro-5H- | | | | | |
| | dibenzo[a,d][7]annulene- | | | | | |
| | 3-carbonyl)-L-methionine | | | | | |
| 98a | methyl N2-(((9H-fluoren-9- | 56 | Nα-Fmoc-L-lysin | C | C | n.q. |
| | yl)methoxy)carbonyl)-N6-(8-((2,4- | | methyl ester | | | |
| | difluorophenyl)amino)- | | hydrochloride | | | |
| | 5-oxo-10,11-dihydro-5H- | | | | | |
| | dibenzo[a,d][7]annulene- | | | | | |
| | 3-carbonyl)-L-lysinate | | | | | |
| 98b | methyl N6-(8-((2,4-difluorophenyl)amino)- | 98a | — | J | — | 55 |
| | 5-oxo-10,11-dihydro-5H- | | | | | |
| | dibenzo[a,d][7]annulene- | | | | | |
| | 3-carbonyl)-L-lysinate | | | | | |
| 98c | N6-(8-((2,4-difluorophenyl)amino)- | 98b | — | B | — | 75 |
| | 5-oxo-10,11-dihydro-5H- | | | | | |
| | dibenzo[a,d][7]annulene- | | | | | |
| | 3-carbonyl)-L-lysine | | | | | |
| 99a | methyl (S)-2-((tert- | 56 | (S)-3-amino-2-((tert.- | C | C | n.q. |
| | butoxycarbonyl)amino)-3-(8-((2,4- | | butoxycarbonyl)amino)propionic | | | |
| | difluorophenyl)amino)-5-oxo-10,11- | | acid | | | |
| | dihydro-5H- | | | | | |
| | dibenzo[a,d][7]annulene- | | | | | |
| | 3-carboxamido)propanoate | | | | | |
| 99b | methyl (S)-2-amino-3-(8-((2,4- | 99a | methanol | I | B | 24 |
| | difluorophenyl)amino)- | | | | | |
| | 5-oxo-10,11-dihydro-5H- | | | | | |
| | dibenzo[a,d][7]annulene- | | | | | |
| | 3-carboxamido)propanoate | | | | | |
| 100a | (S)-2-((tert- | 56 | (S)-4-amino-2-((tert.- | C | C | n.q. |
| | butoxycarbonyl)amino)-4-(8-((2,4- | | butoxycarbonyl)amino)butanoic | | | |
| | difluorophenyl)amino)-5-oxo-10,11- | | acid | | | |
| | dihydro-5H- | | | | | |
| | dibenzo[a,d][7]annulene- | | | | | |
| | 3-carboxamido)butanoic acid | | | | | |
| 100b | methyl (S)-2-amino-4-(8- | 100a | methanol | I | B | 25 |
| | ((2,4-difluorophenyl)amino)- | | | | | |
| | 5-oxo-10,11-dihydro-5H- | | | | | |
| | dibenzo[a,d][7]annulene- | | | | | |
| | 3-carboxamido)butanoate | | | | | |
| 101 | 8-((2-aminophenyl)amino)- | 60 | N,N-dimethyl | C | C | 60 |
| | N-(2-(dimethylamino)ethyl)- | | ethylendiamine | | | |
| | 5-oxo-10,11-dihydro-5H- | | | | | |
| | dibenzo[a,d][7]annulene- | | | | | |
| | 3-carboxamide | | | | | |
| 102 | 8-((2-aminophenyl)amino)- | 60 | 2-phenyl | C | C | 24 |
| | 5-oxo-N-phenethyl-10,11- | | ethylamine | | | |
| | dihydro-5H- | | | | | |
| | dibenzo[a,d][7]annulene- | | | | | |
| | 3-carboxamide | | | | | |
| 103 | 8-((2-aminophenyl)amino)- | 60 | 2-morpholino | C | C | 70 |
| | N-(2-morpholinoethyl)-5- | | ethylamine | | | |
| | oxo-10,11-dihydro-5H- | | | | | |
| | dibenzo[a,d][7]annulene- | | | | | |
| | 3-carboxamide | | | | | |
| 104 | N-(2-(1H-imidazol-5-yl)ethyl)- | 60 | histamine | C | C | 46 |
| | 8-((2-aminophenyl)amino)- | | dihydrochloride | | | |
| | 5-oxo-10,11-dihydro-5H- | | | | | |
| | dibenzo[a,d][7]annulene- | | | | | |
| | 3-carboxamide | | | | | |
| 105a | tert-butyl 4-(2-(8- | 60 | 4-((2- | C | C | 46 |
| | ((2-aminophenyl)amino)- | | aminoethyl)piperazine | | | |
| | 5-oxo-10,11-dihydro-5H- | | carboxylic acid | | | |
| | dibenzo[a,d][7]annulene- | | tert.- butylester | | | |
| | 3-carboxamido)ethyl)piperazine- | | | | | |
| | 1-carboxylate | | | | | |
| 105b | 8-((2-aminophenyl)amino)- | 105a | — | I | A | 67 |
| | 5-oxo-N-(2-(piperazin-1- | | | | | |
| | yl)ethyl)-10,11-dihydro-5H- | | | | | |
| | dibenzo[a,d][7]annulene- | | | | | |
| | 3-carboxamide | | | | | |
| 106 | 8-((2-aminophenyl)amino)- | 60 | ethanolamine | C | C | 13 |
| | N-(2-hydroxyethyl)-5-oxo- | | | | | |
| | 10,11-dihydro-5H- | | | | | |
| | dibenzo[a,d][7]annulene- | | | | | |
| | 3-carboxamide | | | | | |
| 107 | 8-((2-aminophenyl)amino)-N-(2,3- | 60 | 3-aminopropan- | C | C | 21 |
| | dihydroxypropyl)-5-oxo-10,11-dihydro-5H- | | 1,2-diol | | | |
| | dibenzo[a,d][7]annulene- | | | | | |
| | 3-carboxamide | | | | | |
| 108 | 8-((2-aminophenyl)amino)-N-(1,3- | 60 | 2-aminopropan- | C | C | 23 |
| | dihydroxypropan-2-yl)-5-oxo-10,11- | | 1,3-diol | | | |
| | dihydro-5H- | | | | | |
| | dibenzo[a,d][7]annulene- | | | | | |
| | 3-carboxamide | | | | | |
| 109 | 8-((2-aminophenyl)amino)-5-oxo-N- | 60 | D-glucosamine | C | C | 12 |
| | ((2R,3R,4R,5S,6R)-2,4,5-trihydroxy-6- | | hydrochloride | | | |
| | (hydroxymethyl)tetrahydro-2H-pyran-3- | | | | | |
| | yl)-10,11-dihydro-5H- | | | | | |
| | dibenzo[a,d][7]annulene- | | | | | |
| | 3-carboxamide | | | | | |
| 110 | 8-((2-aminophenyl)amino)- | 60 | ammonia (conc. | C | C | 62 |
| | 5-oxo-10,11-dihydro-5H- | | aq. solution) | | | |
| | dibenzo[a,d][7]annulene- | | | | | |
| | 3-carboxamide | | | | | |
| 111 | 8-((2-aminophenyl)amino)- | 117 | — | G | — | 69 |
| | N-methyl-5-oxo-10,11-dihydro-5H- | | | | | |
| | dibenzo[a,d][7]annulene- | | | | | |
| | 3-carboxamide | | | | | |
| 112 | N-(2-aminoethyl)-8-((2- | 60 | ethylen-1,2- | C | C | 22 |
| | aminophenyl)amino)-5-oxo- | | diamine | | | |
| | 10,11-dihydro-5H- | | | | | |
| | dibenzo[a,d][7]annulene- | | | | | |
| | 3-carboxamide | | | | | |
| 113a | methyl 3-(8-((2-aminophenyl)amino)- | 60 | β-alanin methyl | C | C | 99 |
| | 5-oxo-10,11-dihydro-5H- | | ester hydrochloride | | | |
| | dibenzo[a,d][7]annulene- | | | | | |
| | 3-carboxamido)propanoate | | | | | |
| 113b | 3-(8-((2-aminophenyl)amino)- | 113a | — | B | — | 85 |
| | 5-oxo-10,11-dihydro-5H- | | | | | |
| | dibenzo[a,d][7]annulene- | | | | | |
| | 3-carboxamido)propanoic acid | | | | | |
| 114a | dimethyl (8-((2-aminophenyl)amino)- | 60 | L-glutamic acid | C | C | 65 |
| | 5-oxo-10,11-dihydro-5H- | | dimethylester | | | |
| | dibenzo[a,d][7]annulene- | | hydrochloride | | | |
| | 3-carbonyl)-L-glutamate | | | | | |
| 114b | (8-((2-aminophenyl)amino)- | 114 | — | B | — | 84 |
| | 5-oxo-10,11-dihydro-5H- | | | | | |
| | dibenzo[a,d][7]annulene- | | | | | |
| | 3-carbonyl)-L-glutamic acid | | | | | |
| 115 | methyl (8-((2-aminophenyl)amino)- | 60 | L-methionin | C | C | 12 |
| | 5-oxo-10,11-dihydro-5H- | | methyl ester | | | |
| | dibenzo[a,d][7]annulene- | | hydrochloride | | | |
| | 3-carbonyl)-L-methioninate | | | | | |
| 116a | methyl N2-(((9H-fluoren-9- | 60 | Nα-Fmoc-L-lysin | C | C | n.q. |
| | yl)methoxy)carbonyl)-N6-(8-((2- | | methyl ester | | | |
| | aminophenyl)amino)-5-oxo-10,11- | | hydrochloride | | | |
| | dihydro-5H- | | | | | |
| | dibenzo[a,d][7]annulene- | | | | | |
| | 3-carbonyl)-L-lysinate | | | | | |
| 116b | methyl N6-(8-((2-aminophenyl)amino)- | 116a | — | J | — | 94 |
| | 5-oxo-10,11-dihydro-5H- | | | | | |
| | dibenzo[a,d][7]annulene- | | | | | |
| | 3-carbonyl)-L-lysinate | | | | | |
| 116c | N6-(8-((2-aminophenyl)amino)- | 116b | — | B | — | 31 |
| | 5-oxo-10,11-dihydro-5H- | | | | | |
| | dibenzo[a,d][7]annulene- | | | | | |
| | 3-carbonyl)-L-lysine | | | | | |
| 117 | N-methyl-8-((2-nitrophenyl)amino)- | 62 | methylamine (2N | C | C | 87 |
| | 5-oxo-10,11-dihydro-5H- | | in THF) | | | |
| | dibenzo[a,d][7]annulene- | | | | | |
| | 3-carboxamide | | | | | |
| 118 | N-(2-morpholinoethyl)-8- | 62 | 2- | C | C | 82 |
| | ((2-nitrophenyl)amino)- | | morpholinoethylamine | | | |
| | 5-oxo-10,11-dihydro-5H- | | | | | |
| | dibenzo[a,d][7]annulene- | | | | | |
| | 3-carboxamide | | | | | |
| 119 | N-(2-(dimethylamino)ethyl)- | 62 | N,N-dimethyl | C | C | 90 |
| | 8-((2-nitrophenyl)amino)- | | ethylen diamine | | | |
| | 5-oxo-10,11-dihydro-5H- | | | | | |
| | dibenzo[a,d][7]annulene- | | | | | |
| | 3-carboxamide | | | | | |

### Structures and Spectroscopical Data

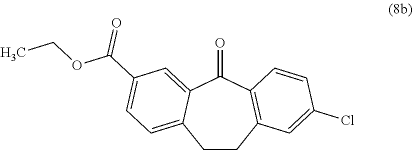 [SMILES: CCOC(=O)c1ccc2c(c1)C(=O)c1ccc(Cl)cc1CC2]

C₁₇H₁₃ClO₄ (M=316.05 g/mol)

Melting point 142.0° C.

Masse (FABₚₒₛ) (m/z): 317.1 [M+H]⁺

IR (ATR) [cm⁻¹] 2970, 2929, 2847, 1718, 1590, 1373, 1640, 1253, 1424, 1195, 1017, 882, 763, 723

¹H-NMR (400 MHz, DMSO-d6) δ 8.38-8.01 (m, 3H), 7.74 (d, J=7.6 Hz, 1H), 7.28 (d, J=7.3 Hz, 2H), 5.42 (s, 2H), 4.35 (dd, J=13.6, 7.1 Hz, 2H), 1.34 (t, J=6.7 Hz, 3H).

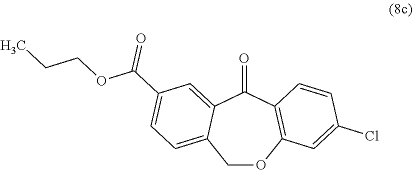 [SMILES: CCCOC(=O)c1ccc2c(c1)C(=O)c1ccc(Cl)cc1OC2]

C₁₈H₁₅ClO₄ (M=330.8 g/mol)

Masse (FABₚₒₛ) (m/z): 331.1 [M+H]⁺

¹H-NMR (400 MHz, DMSO-d6) δ 8.32 (d, J=1.2 Hz, 1H), 8.21 (dt, J=7.9, 3.9 Hz, 1H), 8.12 (d, J=9.1 Hz, 1H), 7.75 (d, J=7.9 Hz, 1H), 7.29 (dd, J=13.2, 11.3 Hz, 2H), 5.43 (s, 2H), 4.25 (dd, J=13.6, 6.9 Hz, 3H), 1.74 (dd, J=14.1, 7.0 Hz, 2H), 0.97 (t, J=7.4 Hz, 3H).

¹³C-NMR (100 MHz, DMSO-d6) δ 188.05, 164.72, 161.28, 140.25, 139.95, 139.58, 133.41, 133.22, 130.74, 129.67, 129.17, 123.81, 122.83, 120.33, 72.66, 66.56, 21.52, 10.25.

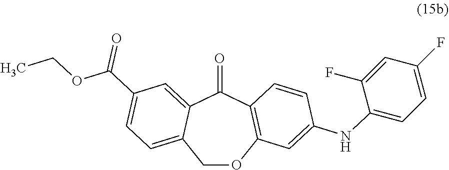 [SMILES: CCOC(=O)c1ccc2c(c1)C(=O)c1ccc(Nc3ccc(F)cc3F)cc1OC2]

C₂₃H₁₇F₂NO₄ (M=409.11 g/mol)

Melting point 83.6° C.

Masse (HRMS-EI) (m/z): 410.1213 (berechnet: 410.1198) [M+H]⁺

IR (ATR) [cm⁻¹] 3315, 2925, 2847, 1614, 1262, 1244, 1119, 1019, 816, 712, 643 ¹H-NMR (400 MHz, DMSO-d6) δ 8.79 (s, 1H), 8.36 (s, 1H), 8.13 (t, J=15.3 Hz, 1H), 8.04 (t, J=11.6 Hz, 1H), 7.68 (d, J=7.6 Hz, 1H), 7.51-7.25 (m, 2H), 7.12 (t, J=7.9 Hz, 1H), 6.64 (d, J=8.5 Hz, 1H), 6.26 (s, 1H), 5.27 (s, 2H), 4.35 (dd, J=13.3, 6.3 Hz, 2H), 1.34 (t, J=6.9 Hz, 3H).

¹³C-NMR (100 MHz, DMSO-d6) δ 186.04, 164.91, 162.86, 152.19, 140.55, 140.07, 133.48, 132.53, 130.45, 129.73, 126.89, 116.48, 109.87, 105.01, 101.75, 72.47, 61.09.

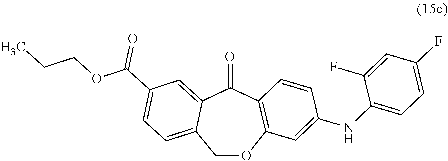 [SMILES: CCCOC(=O)c1ccc2c(c1)C(=O)c1ccc(Nc3ccc(F)cc3F)cc1OC2]

C₂₅H₂₁F₂NO₃ (M=423.13 g/mol)

Melting point 60.1° C.

Masse (HRMS-EI) (m/z): 424.1357 (berechnet: 424.1355) [M+H]⁺

IR (ATR) [cm⁻¹] 3317, 2953, 2353, 2336, 1570, 1508, 1230, 1116, 844, 763, 527

¹H-NMR (400 MHz, DMSO-d6) δ 8.80 (s, 1H), 8.36 (s, 1H), 8.15 (dd, J=7.8, 1.4 Hz, 1H), 8.02 (d, J=9.0 Hz, 1H), 7.68 (d, J=7.8 Hz, 1H), 7.47-7.34 (m, 2H), 7.12 (s, 1H), 6.64 (dd, J=8.9, 1.3 Hz, 1H), 6.26 (s, 1H), 5.27 (s, 2H), 4.26 (t, J=6.5 Hz, 2H), 1.74 (dd, J=14.1, 7.0 Hz, 2H), 0.97 (t, J=7.4 Hz, 3H).

¹³C-NMR (100 MHz, DMSO-d6) δ 186.03, 164.95, 162.87, 152.18, 140.56, 140.09, 133.48, 132.53, 130.42, 129.71, 128.77, 126.89, 126.80, 124.33, 116.46, 112.04, 111.82, 109.87, 105.25, 104.98, 104.74, 101.73, 72.46, 66.46, 21.53, 10.26.

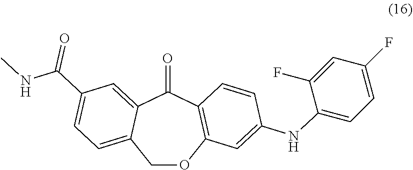 [SMILES: CNC(=O)c1ccc2c(c1)C(=O)c1ccc(Nc3ccc(F)cc3F)cc1OC2]

C₂₂H₁₆F₂N₂O₃ (M=394.11 g/mol)

Melting point 80.1° C.

Masse (HRMS-EI) (m/z): 395.1206 (berechnet: 395.1202) [M+H]⁺

IR (ATR) [cm⁻¹] 3280, 2922, 2852, 1607, 1505, 1260, 1289, 1121, 966, 844

¹H-NMR (400 MHz, DMSO-d6) δ 8.78 (s, 1H), 8.64 (d, J=3.8 Hz, 1H), 8.27 (s, 1H), 8.02 (d, J=8.9 Hz, 2H), 7.62 (t, J=11.1 Hz, 1H), 7.50-7.33 (m, 2H), 7.19-7.00 (m, 1H), 6.92 (s, 1H), 6.64 (d, J=8.9 Hz, 1H), 6.26 (s, 1H), 5.24 (s, 2H), 2.79 (d, J=3.8 Hz, 3H).

¹³C-NMR (100 MHz, DMSO-d6) δ 186.67, 165.60, 162.78, 152.03, 139.92, 138.35, 135.00, 133.37, 130.72, 128.22, 127.71, 126.80, 119.41, 116.57, 111.99, 109.75, 105.24, 104.99, 101.74, 72.43, 26.26.

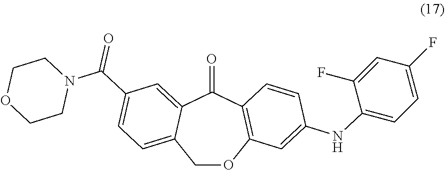 [SMILES: O=C1c2cc(C(=O)N3CCOCC3)ccc2COc2cc(Nc3ccc(F)cc3F)ccc21]

C₂₅H₂₀F₂N₂O₄ (M=450.14 g/mol)

Melting point 115.6° C.

Masse (HRMS-EI) (m/z): 451.1466 (berechnet: 451.1464) [M+H]⁺

IR (ATR) [cm⁻¹] 3284, 2924, 2843, 2356, 1606, 1504, 1245, 1111, 1026, 843, 456

¹H-NMR (400 MHz, DMSO-d6) δ 8.79 (s, 1H), 8.01 (d, J=8.8 Hz, 1H), 7.79 (s, 1H), 7.62 (dd, J=17.3, 7.2 Hz, 2H), 7.50-7.27 (m, 2H), 7.11 (t, J=7.9 Hz, 1H), 6.64 (d, J=8.7 Hz, 1H), 6.26 (s, 1H), 5.23 (s, 2H), 3.62 (s, 8H).

¹³C-NMR (100 MHz, DMSO-d6) δ 186.39, 168.06, 162.84, 152.11, 139.98, 137.07, 136.18, 133.42, 130.66, 128.28, 127.54, 126.76 (d, J=9.8 Hz), 124.33 (d, J=12.3 Hz), 116.55, 111.97, 111.75, 109.77, 105.21, 104.95, 104.71, 101.76, 72.48, 66.00, 57.82.

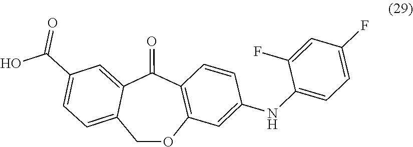 [SMILES: O=C(O)c1ccc2c(c1)C(=O)c1ccc(Nc3ccc(F)cc3F)cc1OC2]

C₂₂H₁₃F₂NO₄ (M=381.08 g/mol)

Melting point 224.2° C.

Masse (HRMS-EI) (m/z): 382.0887 (berechnet: 382.0885) [M+H]⁺

IR (ATR) [cm⁻¹] 3181, 2926, 2852, 1601, 1553, 1274, 1199, 1017, 770, 727, 533

¹H-NMR (400 MHz, DMSO-d6) δ 11.64 (s, 1H), 8.78 (s, 1H), 8.32 (s, 1H), 8.05 (dd, J=21.2, 8.2 Hz, 2H), 7.50 (t, J=10.9 Hz, 1H), 7.47-7.31 (m, 2H), 7.11 (t, J=8.1 Hz, 1H), 6.63 (d, J=8.9 Hz, 1H), 6.26 (s, 1H), 5.22 (s, 2H).

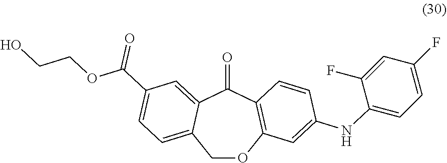 [SMILES: O=C(OCCO)c1ccc2c(c1)C(=O)c1ccc(Nc3ccc(F)cc3F)cc1OC2]

C₂₃H₁₇F₂NO₅ (M=425.11 g/mol)

Melting point 127.2° C.

Masse (HRMS-EI) (m/z): 426.1146 (berechnet: 426.1148) [M+H]⁺

IR (ATR) [cm⁻¹] 3297, 2917, 2855, 2357, 1714, 1567, 1504, 1240, 1120, 965, 763, 525, 545

¹H-NMR (400 MHz, DMSO-d6) δ 8.81 (s, 1H), 8.40 (s, 1H), 8.19 (d, J=7.4 Hz, 1H), 8.03 (d, J=8.8 Hz, 1H), 7.69 (d, J=7.6 Hz, 1H), 7.41 (dt, J=18.7, 9.2 Hz, 2H), 7.12 (t, J=7.4 Hz, 1H), 6.65 (d, J=8.7 Hz, 1H), 6.29 (d, J=24.4 Hz, 1H), 5.28 (s, 2H), 4.99 (s, 1H), 4.31 (s, 2H), 3.89-3.58 (m, 2H).

¹³C NMR (100 MHz, DMSO-d6) δ 186.11, 165.05, 162.85, 152.18, 140.53, 140.08, 133.46, 132.68, 130.43, 129.88, 128.69, 126.90, 126.80, 116.47, 112.04, 111.82, 109.87, 105.25, 105.00, 104.74, 101.74, 72.46, 66.90, 58.99.

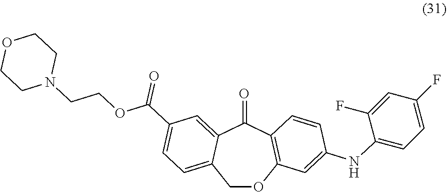 [SMILES: O=C(OCCN1CCOCC1)c1ccc2c(c1)C(=O)c1ccc(Nc3ccc(F)cc3F)cc1OC2]

C₂₇H₂₄F₂N₂O₅ (M=494.17 g/mol)

Melting point 136.2° C.

Masse (HRMS-EI) (m/z): 495.1731 (berechnet: 495.1726) [M+H]⁺

IR (ATR) [cm⁻¹] 3311, 2856, 2359, 1715, 1607, 1505, 1306, 1257, 1115, 1032, 966, 947

¹H-NMR (400 MHz, DMSO-d6) δ 8.81 (s, 1H), 8.35 (s, 1H), 8.13 (dd, J=10.4, 9.1 Hz, 1H), 8.04 (t, J=12.5 Hz, 1H), 7.67 (t, J=12.7 Hz, 1H), 7.49-7.33 (m, 2H), 7.12 (t, J=7.8 Hz, 1H), 6.64 (d, J=8.9 Hz, 1H), 6.26 (s, 1H), 5.27 (s, 2H), 4.42 (t, J=5.3 Hz, 2H), 3.55 (s, 4H), 2.68 (dd, J=18.5, 13.3 Hz, 2H), 2.46 (s, 4H).

¹³C-NMR (100 MHz, DMSO-d6) δ 186.01, 164.87, 162.87, 152.19, 140.62, 140.07, 133.50, 132.57, 130.34, 129.80, 128.79, 126.89, 124.20, 116.45, 112.01, 111.83, 109.87, 105.25, 105.01, 104.75, 101.72, 72.46, 66.17, 62.28, 56.39, 53.32.

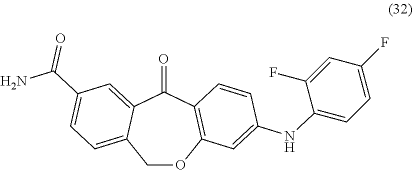 [SMILES: NC(=O)c1ccc2c(c1)C(=O)c1ccc(Nc3ccc(F)cc3F)cc1OC2]

C₂₁H₁₄F₂N₂O₃ (M=380.10 g/mol)

Melting point 266.1° C.

Masse (HRMS-EI) (m/z): 381.1049 (berechnet: 381.1045) [M+H]⁺

IR (ATR) [cm⁻¹] 3354, 3280, 1634, 1568, 1505, 1417, 1276, 1118, 1097, 967

¹H-NMR (400 MHz, DMSO-d6) δ 8.76 (d, J=4.1 Hz, 1H), 8.30 (d, J=2.9 Hz, 1H), 8.16 (s, 1H), 8.04 (ddd, J=18.5, 8.2, 5.2 Hz, 2H), 7.60 (dd, J=7.6, 4.5 Hz, 1H), 7.50 (s, 1H), 7.46-7.32 (m, 2H), 7.11 (dd, J=7.6, 4.8 Hz, 1H), 6.71-6.59 (m, 1H), 6.26 (s, 1H), 5.24 (d, J=4.1 Hz, 2H).

¹³C-NMR (100 MHz, DMSO-d6) δ 186.73, 166.96, 162.79, 152.03, 139.94, 138.51, 134.87, 133.36, 131.06, 128.21, 128.14, 126.80, 126.71, 124.39, 116.61, 112.01, 111.79, 109.75, 105.22, 104.98, 104.71, 101.78, 72.44.

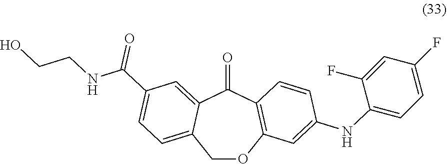 [SMILES: O=C(NCCO)c1ccc2c(c1)C(=O)c1ccc(Nc3ccc(F)cc3F)cc1OC2]

C₂₃H₁₈F₂N₂O₄ (M=424.12 g/mol)

Melting point 170.1° C.

Masse (HRMS-EI) (m/z): 425.1310 (berechnet: 425.1307) [M+H]⁺

IR (ATR) [cm⁻¹] 3271, 2918, 1651, 1608, 1567, 1505, 1439, 1253, 1127, 1023, 967

¹H-NMR (400 MHz, DMSO-d6) δ 8.76 (s, 1H), 8.63 (s, 1H), 8.29 (s, 1H), 8.03 (dd, J=14.0, 5.2 Hz, 2H), 7.63-7.58 (m, 1H), 7.47-7.34 (m, 3H), 7.10 (t, J=17.8 Hz, 1H), 6.62 (t, J=14.0 Hz, 1H), 6.23 (d, J=24.2 Hz, 1H), 5.24 (s, 2H), 4.75 (d, J=2.8 Hz, 1H), 3.52 (d, J=2.8 Hz, 2H), 3.37 (s, 2H).

¹³C NMR (100 MHz, DMSO-d6) δ 186.73, 165.36, 162.78, 159.54, 152.02, 139.89, 138.35, 135.08, 133.36, 130.89, 128.16, 127.84, 126.80, 126.70, 124.27, 116.57, 112.02, 111.79, 109.74, 105.23, 104.96, 104.72, 101.74, 72.42, 59.64, 42.26.

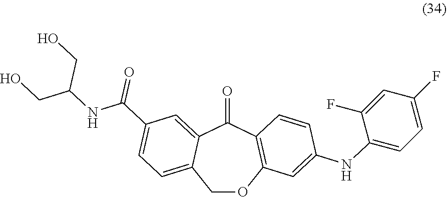 [SMILES: O=C(NC(CO)CO)c1ccc2c(c1)C(=O)c1ccc(Nc3ccc(F)cc3F)cc1OC2]

C₂₄H₂₀F₂N₂O₅ (M=454.13 g/mol)

Melting point 199.3° C.

Masse (HRMS-EI) (m/z): 455.1409 (berechnet: 455.1413) [M+H]⁺

IR (ATR) [cm⁻¹] 3273, 2923, 1603, 1568, 1504, 1381, 1259, 1228, 1122, 1032, 966

¹H-NMR (400 MHz, DMSO-d6) δ 8.77 (s, 1H), 8.28 (d, J=19.9 Hz, 1H), 8.21 (d, J=8.0 Hz, 1H), 8.14-7.93 (m, 2H), 7.61 (d, J=7.8 Hz, 1H), 7.50-7.32 (m, 2H), 7.11 (t, J=7.8 Hz, 1H), 6.64 (d, J=9.0 Hz, 1H), 6.26 (s, 1H), 5.24 (s, 2H), 4.69 (s, 2H), 4.05-3.88 (m, 1H), 3.52 (s, 4H).

¹³C-NMR (100 MHz, DMSO-d6) δ 186.85, 165.32, 162.77, 152.02, 139.87, 138.26, 135.30, 133.32, 131.08, 128.06, 127.93, 126.79, 126.73, 116.58, 112.01, 111.80, 109.73, 105.23, 104.98, 104.72, 101.75, 72.42, 60.37, 54.03.

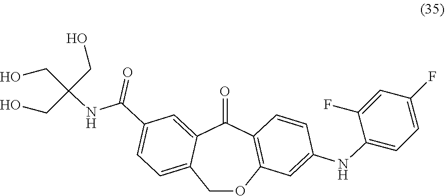 [SMILES: O=C(NC(CO)(CO)CO)c1ccc2c(c1)C(=O)c1ccc(Nc3ccc(F)cc3F)cc1OC2]

C₂₅H₂₂F₂N₂O₆ (M=484.14 g/mol)

Melting point 171.9° C.

Masse (FABₚₒₛ) (m/z): 485.4 [M+H]⁺

IR (ATR) [cm⁻¹] 3288, 1607, 1578, 1520, 1440, 1260, 1229, 1120, 1036, 966

¹H-NMR (400 MHz, DMSO-d6) δ 8.76 (d, J=11.5 Hz, 1H), 8.20 (d, J=11.8 Hz, 1H), 8.01 (dd, J=14.4, 5.2 Hz, 2H), 7.60 (d, J=6.8 Hz, 1H), 7.53-7.30 (m, 3H), 7.11 (t, J=8.4 Hz, 1H), 6.64 (d, J=8.9 Hz, 1H), 6.24 (d, J=11.4 Hz, 1H), 5.23 (d, J=11.8 Hz, 2H), 4.76 (s, 3H), 3.70 (s, 6H).

¹³C-NMR (100 MHz, DMSO-d6) δ 186.76, 166.35, 162.79, 152.03, 139.77, 138.33, 135.85, 133.35, 131.06, 128.05, 127.97, 126.72, 124.22, 116.55, 112.00, 111.78, 109.73, 105.24, 104.97, 104.73, 101.72, 72.41, 62.85, 60.28.

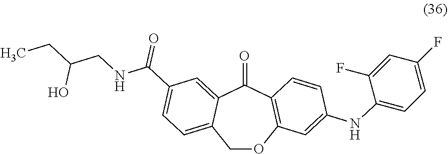 [SMILES: CCC(O)CNC(=O)c1ccc2c(c1)C(=O)c1ccc(Nc3ccc(F)cc3F)cc1OC2]

C₂₅H₂₂F₂N₂O₄ (M=452.15 g/mol)

Melting point 183.9° C.

Masse (FABₚₒₛ) (m/z): 453.3 [M+H]⁺

IR (ATR) [cm⁻¹] 3283, 2924, 1607, 1568, 1505, 1437, 1259, 1228, 1121, 1094, 966

¹H-NMR (400 MHz, DMSO-d6) δ 8.78 (s, 1H), 8.60 (s, 1H), 8.30 (s, 1H), 8.04 (t, J=10.0 Hz, 2H), 7.61 (d, J=7.7 Hz, 1H), 7.48-7.32 (m, 2H), 7.11 (t, J=7.9 Hz, 1H), 6.65 (d, J=8.8 Hz, 1H), 6.26 (s, 1H), 5.24 (s, 2H), 4.74 (s, 1H), 3.55 (s, 1H), 3.34-3.14 (m, 2H), 1.52-1.25 (m, 2H), 0.89 (t, J=6.9 Hz, 3H).

¹³C-NMR (100 MHz, DMSO-d6) δ 186.77, 165.38, 162.79, 159.96, 157.65, 157.10, 156.97, 154.63, 154.50, 151.98, 139.89, 138.32, 135.11, 133.36, 130.96, 128.23, 127.97, 127.79, 126.73, 124.34, 124.22, 116.52, 111.88, 109.83, 109.62, 105.10, 101.71, 72.39, 70.16, 45.59, 27.38, 10.02.

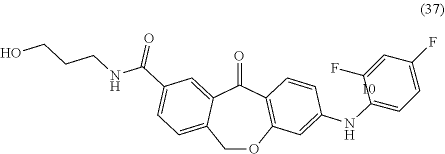 [SMILES: O=C(NCCCO)c1ccc2c(c1)C(=O)c1ccc(Nc3ccc(F)cc3F)cc1OC2]

C₂₄H₂₄F₂N₂O₄ (M=438.14 g/mol)

Melting point 158.3° C.

Masse (HRMS-EI) (m/z): 439.1438 (berechnet: 439.1464) [M+H]⁺

IR (ATR) [cm⁻¹] 3279, 2923, 2853, 1608, 1504, 1438, 1382, 1260, 1123 1095, 966

¹H-NMR (400 MHz, DMSO-d6) δ 8.78 (s, 1H), 8.62 (d, J=19.9 Hz, 1H), 8.25 (d, J=19.6 Hz, 1H), 8.02 (d, J=8.6 Hz, 2H), 7.95 (s, 1H), 7.61 (d, J=7.8 Hz, 2H), 7.41 (dt, J=18.9, 9.3 Hz, 2H), 7.12 (t, J=8.2 Hz, 1H), 6.64 (d, J=8.8 Hz, 1H), 6.26 (s, 1H), 5.27 (d, J=28.5 Hz, 2H), 4.53 (d, J=29.3 Hz, 1H), 3.46 (d, J=4.8 Hz, 2H), 3.32 (d, J=6.2 Hz, 2H), 1.69 (dd, J=12.9, 6.4 Hz, 2H).

¹³C-NMR (100 MHz, DMSO-d6) δ 186.74, 165.19, 162.79, 151.99, 139.91, 138.32, 135.10, 133.37, 130.85, 128.14, 127.83, 127.67, 124.22, 116.51, 109.82, 105.12, 101.70, 72.38, 58.54, 36.64, 32.26.

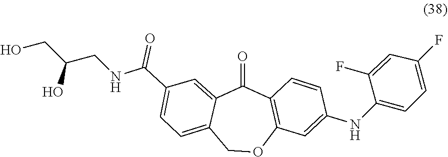 [SMILES: O=C(NC[C@@H](O)CO)c1ccc2c(c1)C(=O)c1ccc(Nc3ccc(F)cc3F)cc1OC2]

C₂₄H₁₉F₂N₂O₅ (M=454.13 g/mol)

Melting point 188.7° C.

Masse (HRMS-EI) (m/z): 455.1413 (berechnet: 455.1413) [M+H]⁺

IR (ATR) [cm⁻¹] 3289, 2724, 1607, 1505, 1383, 1260 1229, 1122, 1095, 966

¹H-NMR (400 MHz, DMSO-d6) δ 8.77 (s, 1H), 8.60 (t, J=5.2 Hz, 1H), 8.29 (s, 1H), 8.09-7.95 (m, 2H), 7.61 (d, J=7.8 Hz, 1H), 7.48-7.31 (m, 2H), 7.12 (t, J=7.5 Hz, 1H), 6.64 (d, J=8.8 Hz, 1H), 6.26 (s, 1H), 5.24 (s, 2H), 4.83 (s, 1H), 4.59 (s, 1H), 3.74-3.48 (m, 2H), 3.26-3.03 (m, 2H).

¹³C-NMR (100 MHz, DMSO-d6) δ 186.72, 165.63, 162.78, 152.04, 139.89, 138.38, 135.08, 133.36, 130.92, 128.16, 127.89, 126.83, 126.74, 116.59, 112.01, 111.76, 109.76, 105.22, 104.96, 101.77, 72.43, 70.30, 63.95, 43.12.

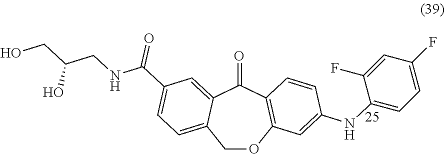 [SMILES: O=C(NC[C@H](O)CO)c1ccc2c(c1)C(=O)c1ccc(Nc3ccc(F)cc3F)cc1OC2]

C₂₄H₁₉F₂N₂O₅ (M=454.13 g/mol)

Melting point 214.1° C.

Masse (HRMS-EI) (m/z): 455.1409 (berechnet: 455.1413) [M+H]⁺

IR (ATR) [cm⁻¹] 3290, 2922, 1632, 1567, 1505, 1381, 1260, 1229, 1122 1033, 966

¹H-NMR (400 MHz, DMSO-d6) δ 8.78 (s, 1H), 8.61 (s, 1H), 8.29 (s, 1H), 8.11-7.98 (m, 2H), 7.61 (d, J=7.8 Hz, 1H), 7.41 (dt, J=18.0, 9.1 Hz, 2H), 7.12 (t, J=7.7 Hz, 1H), 6.64 (d, J=8.8 Hz, 1H), 6.26 (s, 1H), 5.28 (d, J=27.5 Hz, 2H), 3.71-3.56 (m, 2H), 3.25-3.14 (m, 2H).

¹³C-NMR (100 MHz, DMSO-d6) δ 186.72, 165.63, 162.78, 152.04, 139.89, 138.38, 135.08, 133.36, 130.92, 128.16, 127.89, 126.83, 126.74, 116.59, 112.01, 111.76, 109.76, 105.22, 104.96, 101.77, 72.43, 70.30, 63.95, 43.12.

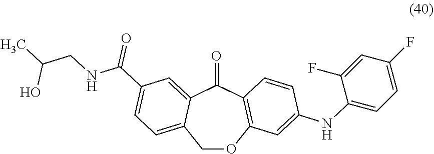 [SMILES: CC(O)CNC(=O)c1ccc2c(c1)C(=O)c1ccc(Nc3ccc(F)cc3F)cc1OC2]

C₂₄H₂₀F₂N₂O₄ (M=438, 14 g/mol)

Melting point 191.5° C.

Masse (HRMS-EI) (m/z): 439.1463 (berechnet: 439.1464) [M+H]⁺

IR (ATR) [cm⁻¹] 3282, 2970, 2925, 1633, 1505, 1378, 1259, 1228, 1121, 1094, 966

¹H-NMR (400 MHz, DMSO-d6) δ 8.78 (s, 1H), 8.63 (d, J=5.0 Hz, 1H), 8.30 (s, 1H), 8.12-7.98 (m, 2H), 7.61 (d, J=7.8 Hz, 1H), 7.47-7.32 (m, 2H), 7.12 (t, J=8.1 Hz, 1H), 6.64 (d, J=8.9 Hz, 1H), 6.26 (s, 1H), 5.24 (s, 2H), 4.73 (s, 1H), 3.80 (dd, J=11.9, 5.9 Hz, 1H), 3.21 (t, J=5.4 Hz, 2H), 1.07 (d, J=6.1 Hz, 3H).

¹³C-NMR (100 MHz, DMSO-d6) δ 186.77, 165.36, 162.79, 154.63, 154.50, 151.99, 139.89, 138.33, 135.11, 133.37, 130.95, 128.16, 127.94, 127.83, 126.74, 124.33, 124.24, 116.52, 110.09, 109.61, 109.37, 105.01, 101.75, 101.64, 72.39, 65.18, 47.21, 21.33.

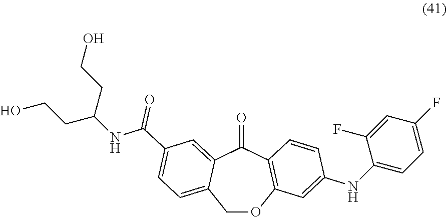 [SMILES: O=C(NC(CCO)CCO)c1ccc2c(c1)C(=O)c1ccc(Nc3ccc(F)cc3F)cc1OC2]

C₂₆H₂₄F₂N₂O₅ (M=482.17 g/mol)

Melting point 181.5° C.

Masse (FABₚₒₛ) (m/z): 483.3 [M+H]⁺

IR (ATR) [cm⁻¹] 3273, 2924, 1607, 1567, 1504, 1438, 1383, 1259, 1229, 1122, 1095, 1034, 965

¹H-NMR (400 MHz, DMSO-d6) δ 8.77 (s, 1H), 8.37 (d, J=8.4 Hz, 1H), 8.31-8.20 (m, 1H), 8.09-7.92 (m, 2H), 7.61 (d, J=7.8 Hz, 1H), 7.40 (dt, J=19.4, 9.6 Hz, 2H), 7.19-7.04 (m, 1H), 6.62 (t, J=17.3 Hz, 1H), 6.26 (s, 1H), 5.24 (s, 2H), 4.43 (s, 2H), 4.15 (dd, J=13.8, 6.8 Hz, 1H), 3.42 (s, 4H), 1.68 (dd, J=12.8, 6.3 Hz, 4H).

¹³C-NMR (100 MHz, DMSO-d6) δ 186.82, 165.23, 162.78, 152.03, 139.93, 138.29, 135.24, 133.32, 130.97, 128.16, 127.72, 126.82, 126.72, 116.55, 112.02, 111.80, 109.74, 105.23, 104.99, 101.73, 72.39, 58.05, 44.13, 37.58.

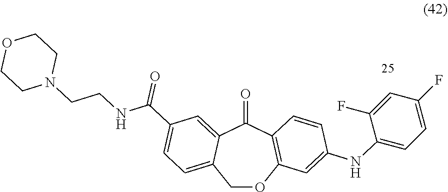 [SMILES: O=C(NCCN1CCOCC1)c1ccc2c(c1)C(=O)c1ccc(Nc3ccc(F)cc3F)cc1OC2]

C₂₇H₂₅F₂N₃O₄ (M=493.18 g/mol)

Masse (HRMS-EI) (m/z): 494.1881 (berechnet: 494.1886) [M+H]⁺

IR (ATR) [cm⁻¹] 2359, 1651, 1644, 1505, 1487, 1248, 1112, 1034, 854, 668

¹H-NMR (400 MHz, DMSO-d6) δ 8.78 (s, 1H), 8.63 (s, 1H), 8.27 (s, 1H), 8.04 (s, 2H), 7.59 (t, J=17.1 Hz, 1H), 7.40 (d, J=8.6 Hz, 2H), 7.12 (s, 1H), 6.61 (t, J=21.1 Hz, 1H), 6.26 (s, 1H), 5.24 (s, 2H), 3.56 (s, 4H), 3.41 (s, 2H), 2.47 (s, 2H), 2.41 (s, 4H).

¹³C-NMR (100 MHz, DMSO-d6) δ 186.72, 165.17, 162.79, 152.04, 139.93, 138.39, 135.07, 133.36, 130.85, 128.22, 127.75, 126.83, 116.57, 112.02, 111.81, 109.75, 105.24, 104.97, 104.73, 101.75, 72.42, 66.17, 57.24, 53.25, 36.62.

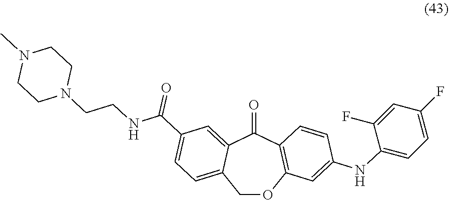 [SMILES: CN1CCN(CCNC(=O)c2ccc3c(c2)C(=O)c2ccc(Nc4ccc(F)cc4F)cc2OC3)CC1]

C₂₈H₂₈F₂N₄O₃ (M=506.21 g/mol)

Melting point 217.2° C.

Masse (HRMS-EI) (m/z): 507.2201 (berechnet: 507.2202) [M+H]⁺

IR (ATR) [cm⁻¹] 3278, 2925, 1633, 1567, 1505, 1455, 1383, 1261, 1123, 1030, 966

¹H-NMR (400 MHz, DMSO-d6) δ 8.80 (s, 1H), 8.61 (s, 1H), 8.27 (s, 1H), 8.02 (d, J=8.9 Hz, 2H), 7.61 (d, J=7.7 Hz, 1H), 7.41 (d, J=9.2 Hz, 2H), 7.14 (d, J=8.2 Hz, 1H), 6.64 (d, J=9.3 Hz, 1H), 6.26 (s, 1H), 5.24 (s, 2H), 3.37 (d, J=6.1 Hz, 2H), 2.31 (s, 2H), 2.13 (s, 3H), 1.88 (s, 8H).

¹³C-NMR (100 MHz, DMSO-d6) δ 186.70, 172.17, 165.12, 162.78, 152.04, 139.92, 138.37, 135.08, 133.34, 130.83, 128.20, 127.74, 126.83, 116.55, 112.02, 111.76, 109.75, 104.99, 101.74, 72.41, 56.79, 54.66, 52.59, 45.65, 36.94,

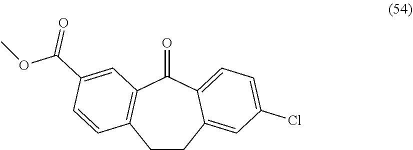 [SMILES: COC(=O)c1ccc2c(c1)C(=O)c1ccc(Cl)cc1CC2]

C₁₇H₁₃ClO₃ (M=300.06 g/mol)

Masse (ESIₚₒₛ) (m/z)=301.3 [M+H]⁺

¹H-NMR (400 MHz, DMSO-d6) δ 8.44 (s, 1H), 8.08-7.98 (m, 1H), 7.94-7.82 (m, 1H), 7.53-7.37 (m, 3H), 3.86 (s, 3H), 3.29-3.11 (m, 4H).

¹³C-NMR (100 MHz, DMSO-d6) δ 192.23, 165.48, 147.31, 144.30, 137.59, 137.49, 136.15, 132.73, 132.34, 131.17, 130.53, 129.21, 128.12, 126.77, 52.27, 33.85, 33.29.

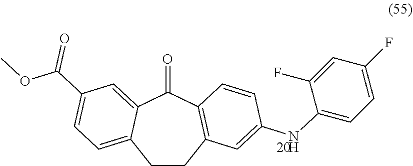 [SMILES: COC(=O)c1ccc2c(c1)C(=O)c1ccc(Nc3ccc(F)cc3F)cc1CC2]

C₂₃H₁₇F₂NO₅ (M=393.12 g/mol)

Masse (HRMS-EI)³⁹ (m/z): 394.1249 (berechnet: 394.1249) [M+H]⁺

¹H-NMR (400 MHz, DMSO-d6)³⁹ δ 8.66 (d, J=1.8 Hz, 1H), 8.16 (d, J=8.7 Hz, 1H), 8.06 (dd, J=1.9 Hz, 1H), 7.25-7.41 (m, 2H), 6.81-6.99 (m, 3H), 6.66 (d, J=2.4 Hz, 1H), 6.00 (s, 1H), 3.92 (s, 3H), 3.06-3.23 (m, 4H)

¹³C-NMR (100 MHz, DMSO-d6)³⁹ δ 191.0, 166.5, 158.8, 155.3, 148.0, 146.4, 145.1, 139.5, 134.1, 132.6, 132.3, 129.2, 129.0, 128.9, 124.3, 123.9, 114.6, 113.3, 111.4, 104.8, 52.1, 35.7,

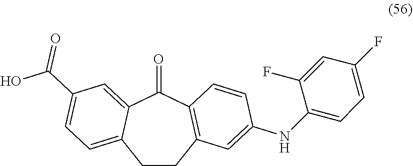 [SMILES: O=C(O)c1ccc2c(c1)C(=O)c1ccc(Nc3ccc(F)cc3F)cc1CC2]

C₂₂H₁₅F₂NO₃ (M=379.10 g/mol)

Masse (HRMS-EI)³⁹ (m/z): 380.1092 (berechnet: 380.1093) [M+H]⁺

¹H-NMR (200 MHz, DMSO-d6)³⁹ 3.03-3.18 (m, 4H), 6.62 (s, 1H), 6.75 (d, 1H), 7.04-7.16 (m, 1H), 7.30-7.49 (m, 3H), 7.95-8.03 (m, 2H), 8.43 (d, J=1.64 Hz, 1H), 8.60 (s, 1H), 13.02 (s, 1H)

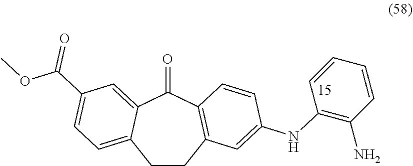 [SMILES: COC(=O)c1ccc2c(c1)C(=O)c1ccc(Nc3ccccc3N)cc1CC2]

C₂₃H₂₀N₂O₃ (M=372.15 g/mol)

Masse (ESI_{neg}) (m/z): 370.9 [M−H]⁻

IR (ATR) [cm⁻¹] 1714, 1602, 1577, 1498, 1353, 1242, 1116, 837, 743, 626

¹H-NMR (400 MHz, DMSO-d6) δ 8.45 (d, J=1.6 Hz, 1H), 8.06 (s, 1H), 7.98 (dd, J=10.6, 5.0 Hz, 2H), 7.43 (d, J=7.9 Hz, 1H), 7.02 (d, J=7.6 Hz, 1H), 6.96 (dd, J=11.1, 4.0 Hz, 1H), 6.79 (d, J=7.9 Hz, 1H), 6.64 (dd, J=8.9, 1.5 Hz, 1H), 6.59 (t, J=7.1 Hz, 1H), 6.47 (d, J=1.3 Hz, 1H), 4.86 (s, 2H), 3.86 (s, 3H), 3.14-3.08 (m, 2H), 3.02-2.94 (m, 2H).

¹³C-NMR (100 MHz, DMSO-d6) δ 189.13, 165.74, 151.09, 146.94, 145.31, 143.65, 139.26, 133.67, 131.79, 131.32, 129.40, 127.94, 126.16, 125.89, 125.48, 124.71, 116.46, 115.52, 112.69, 111.87, 52.12, 35.44, 34.09.

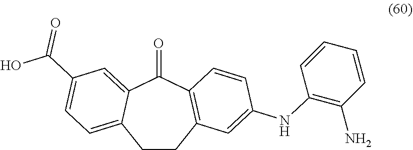 [SMILES: Nc1ccccc1Nc1ccc2c(c1)CCc1ccc(C(=O)O)cc1C2=O]

C₂₂H₁₈N₂O₃ (M=358.13 g/mol)

Masse (ESI_{neg}) (m/z): 357.1 [M−H]⁻

IR (ATR) [cm⁻¹] 1682, 1579, 1505, 1454, 1353, 1239, 1108, 831, 745, 622

¹H-NMR (400 MHz, DMSO-d6) δ 13.04 (s, 1H), 9.70 (s, 1H), 8.44 (s, 1H), 8.26 (s, 1H), 8.01 (d, J=18.4 Hz, 3H), 7.44 (s, 1H), 7.33 (dd, J=17.6, 5.7 Hz, 1H), 7.18 (s, 1H), 7.05-6.90 (m, 1H), 6.74 (dd, J=33.7, 7.4 Hz, 1H), 6.67-6.43 (m, 2H), 3.12 (s, 2H), 3.01 (s, 2H).

¹³C-NMR (100 MHz, DMSO-d6) δ 189.71, 189.33, 166.79, 160.26, 151.03, 146.49, 145.24, 143.67, 139.16, 133.52, 132.34, 131.45, 129.35, 126.13, 125.88, 124.99, 122.95, 116.43, 115.50, 113.60, 112.70, 112.43, 111.85, 35.28, 34.04.

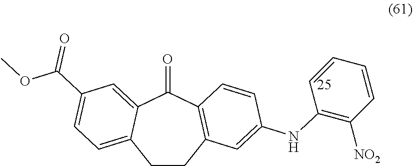 [SMILES: COC(=O)c1ccc2c(c1)C(=O)c1ccc(Nc3ccccc3[N+](=O)[O-])cc1CC2]

C₂₃H₁₈N₂O₅ (M=402.12 g/mol)

Masse (ESI_{neg}) (m/z): 401.1 [M−H]⁻

IR (ATR) [cm⁻¹] 1722, 1601, 1563, 1506, 1435, 1348, 1240, 1156, 838, 761, 640, 539

¹H-NMR (400 MHz, DMSO-d6) δ 8.46 (d, J=1.7 Hz, 1H), 8.12-8.07 (m, 1H), 8.04 (d, J=2.9 Hz, 1H), 8.02-8.00 (m, 1H), 7.66-7.59 (m, 1H), 7.59-7.54 (m, 1H), 7.49 (d, J=8.0 Hz, 1H), 7.20 (dd, J=8.7, 2.1 Hz, 1H), 7.15-7.07 (m, 2H), 3.87 (s, 3H), 3.23-3.16 (m, 2H), 3.13 (d, J=6.1 Hz, 2H).

¹³C-NMR (100 MHz, DMSO-d6) δ 190.77, 165.60, 147.06, 145.24, 144.76, 138.57, 137.67, 137.51, 135.31, 132.86, 132.24, 131.13, 130.61, 129.83, 128.07, 126.06, 121.15, 120.46, 118.91, 116.86, 52.04, 34.52.

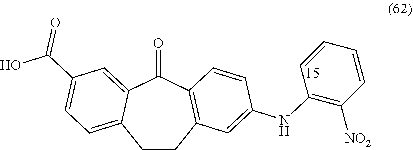 [SMILES: O=C(O)c1ccc2c(c1)C(=O)c1ccc(Nc3ccccc3[N+](=O)[O-])cc1CC2]

C₂₂H₁₆N₂O₅ (M=388.11 g/mol)

Masse (ESI_{neg}) (m/z): 387.0 [M−H]⁻

IR (ATR) [cm⁻¹] 1690, 1598, 1571, 1495, 1345, 1253, 1115, 882, 741, 507

¹H-NMR (200 MHz, DMSO-d6) δ 9.30 (s, 1H), 8.45 (s, 1H), 8.10 (d, J=8.0 Hz, 1H), 8.02 (d, J=8.2 Hz, 2H), 7.58 (s, 2H), 7.48 (d, J=7.7 Hz, 1H), 7.17 (d, J=14.4 Hz, 3H), 3.18 (s, 2H), 3.16-3.08 (m, 2H).

¹³C-NMR (50 MHz, DMSO-d6) δ 191.35, 167.08, 147.09, 145.87, 145.22, 138.91, 138.15, 135.82, 133.32, 131.75, 131.01, 130.17, 129.61, 126.55, 121.75, 121.21, 119.33, 117.29, 35.03, 34.34.

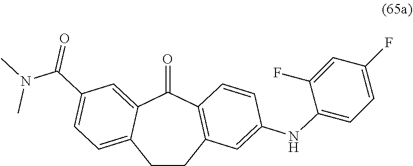 [SMILES: CN(C)C(=O)c1ccc2c(c1)C(=O)c1ccc(Nc3ccc(F)cc3F)cc1CC2]

C₂₄H₂₀F₂N₂O₂ (M=406.15 g/mol)

Masse (ESI_{neg}) (m/z): 404.8 [M−H]⁻

IR (ATR) [cm⁻¹] 3247, 2921, 2852, 1621, 1589, 1500, 1392, 1358, 1257, 1212, 1138, 965, 866, 601

¹H-NMR (400 MHz, DMSO-d6) δ 8.61 (s, 1H), 7.99 (d, J=8.7 Hz, 1H), 7.86 (s, 1H), 7.50 (d, J=7.7 Hz, 1H), 7.48-7.34 (m, 3H), 7.11 (t, J=8.2 Hz, 1H), 6.75 (d, J=8.7 Hz, 1H), 6.63 (s, 1H), 3.11 (d, J=8.3 Hz, 2H), 3.05 (d, J=8.3 Hz, 2H), 2.98 (s, 3H), 2.93 (s, 3H).

¹³C-NMR (100 MHz, DMSO-d6) δ 190.08, 169.46, 149.33, 145.29, 142.94, 138.60, 134.60, 133.43, 130.29, 128.89, 127.13, 126.26, 113.47, 112.26, 104.93, 40.15, 39.94, 39.73, 39.52, 39.52, 39.31, 39.10, 38.89, 35.39, 33.82.

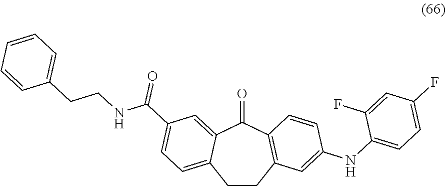 [SMILES: O=C(NCCc1ccccc1)c1ccc2c(c1)C(=O)c1ccc(Nc3ccc(F)cc3F)cc1CC2]

C₃₀H₂₄F₂N₂O₂ (M=482.18 g/mol)

Masse (ESI_{neg}) (m/z): 481.9 [M−H]⁻

IR (ATR) [cm⁻¹] 3283, 2923, 1632, 1338, 1314, 1258, 1141, 965, 851, 599

¹H-NMR (400 MHz, DMSO-d6) δ 8.67 (s, 1H), 8.58 (s, 1H), 8.33 (s, 1H), 8.00 (d, J=9.1 Hz, 1H), 7.90 (d, J=7.3 Hz, 1H), 7.39 (d, J=7.4 Hz, 3H), 7.28 (d, J=7.6 Hz, 5H), 7.13 (d, J=9.3 Hz, 2H), 6.76 (d, J=8.4 Hz, 1H), 6.63 (s, 1H), 3.48 (s, 2H), 3.08 (d, J=5.3 Hz, 4H), 2.87 (d, J=7.2 Hz, 2H).

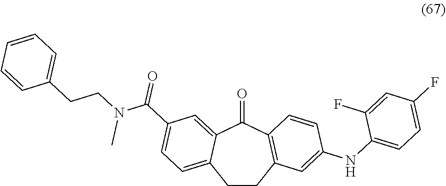 [SMILES: CN(CCc1ccccc1)C(=O)c1ccc2c(c1)C(=O)c1ccc(Nc3ccc(F)cc3F)cc1CC2]

C₃₁H₂₆F₂N₂O₂ (M=496.20 g/mol)

Masse (ESI_{neg}) (m/z): 495.1 [M−H]⁻

IR (ATR) [cm⁻¹] 1602, 1579, 1500, 1397, 1354, 1258, 1214, 1139, 964, 842, 698

¹H-NMR (400 MHz, DMSO-d6) δ 8.60 (s, 1H), 7.99 (d, J=8.8 Hz, 1H), 7.43-7.36 (m, 3H), 7.27 (d, J=22.0 Hz, 3H), 7.17-7.07 (m, 4H), 6.98 (s, 1H), 6.76 (d, J=8.4 Hz, 1H), 6.63 (s, 1H), 3.66 (s, 2H), 3.06 (d, J=5.6 Hz, 3H), 3.04 (d, J=6.5 Hz, 2H), 3.02 (s, 2H), 2.88 (d, J=14.1 Hz, 2H).

¹³C-NMR (100 MHz, DMSO-d6) δ 190.60, 160.24, 157.82, 157.71, 157.47, 157.35, 155.01, 154.88, 149.80, 145.72, 139.93, 139.11, 133.88, 129.17, 129.08, 128.78, 128.71, 127.69, 126.70, 126.64, 125.36, 114.00, 112.77, 112.39, 112.21, 105.67, 105.42, 105.16, 52.72, 51.76, 48.91, 35.92, 34.28.

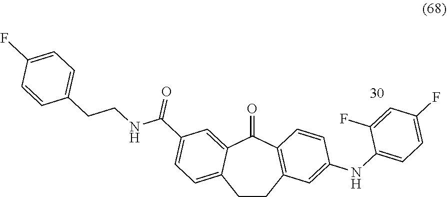 [SMILES: O=C(NCCc1ccc(F)cc1)c1ccc2c(c1)C(=O)c1ccc(Nc3ccc(F)cc3F)cc1CC2]

C₃₀H₂₃F₃N₂O₂(M=500,17 g/mol)

Masse (ESI_{neg}) (m/z): 499.8 [M−H]⁻

IR (ATR) [cm⁻¹] 3280, 2923, 2853, 1602, 1580, 1257, 1139, 1094, 964, 846, 783, 518

¹H-NMR (400 MHz, DMSO-d6) δ 8.68 (s, 1H), 8.60 (s, 1H), 8.31 (s, 1H), 8.00 (d, J=8.5 Hz, 1H), 7.88 (d, J=7.3 Hz, 1H), 7.38 (dd, J=15.4, 8.7 Hz, 4H), 7.09 (t, J=11.2 Hz, 3H), 7.01 (t, J=8.2 Hz, 1H), 6.76 (d, J=8.3 Hz, 1H), 6.62 (s, 1H), 3.51 (d, J=5.9 Hz, 2H), 3.10 (s, 2H), 3.05 (s, 2H), 2.87 (d, J=6.9 Hz, 2H).

¹³C-NMR (100 MHz, DMSO-d6) δ 190.41, 190.05, 165.48, 160.96, 149.29, 145.26, 144.58, 142.53, 138.86, 133.36, 132.75, 130.31, 129.11, 127.08, 126.22, 124.82, 115.40, 115.19, 113.49, 112.92, 112.71, 112.28, 111.94, 104.95, 40.41, 35.26, 33.68, 33.47.

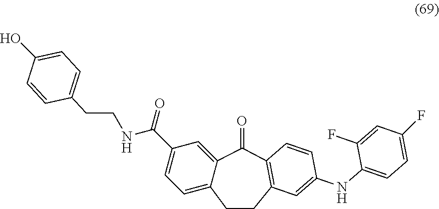 [SMILES: O=C(NCCc1ccc(O)cc1)c1ccc2c(c1)C(=O)c1ccc(Nc3ccc(F)cc3F)cc1CC2]

C₃₀H₂₄F₂N₂O₃ (M=498.18 g/mol)

Masse (ESI_{neg}) (m/z): 497.5 [M−H]⁻

IR (ATR) [cm⁻¹] 3292, 2922, 2852, 1601, 1505, 1360, 1260, 1097, 967, 843, 540

¹H-NMR (400 MHz, DMSO-d6) δ 9.20 (s, 1H), 8.64 (s, 1H), 8.60 (s, 1H), 8.32 (s, 1H), 7.99 (d, J=8.5 Hz, 1H), 7.89 (d, J=7.4 Hz, 1H), 7.46-7.33 (m, 3H), 7.10 (t, J=7.9 Hz, 1H), 7.02 (d, J=7.7 Hz, 2H), 6.76 (d, J=8.4 Hz, 1H), 6.67 (d, J=7.6 Hz, 2H), 6.62 (s, 1H), 3.10 (s, 2H), 3.05 (s, 2H), 2.88 (s, 1H), 2.72 (s, 2H).

¹³C-NMR (100 MHz, DMSO-d6) 190.47, 165.44, 155.56, 149.30, 145.23, 144.50, 138.84, 133.32, 132.92, 130.31, 129.55, 129.44, 129.10, 128.87, 127.17, 115.09, 113.53, 112.28, 111.90, 104.92, 41.20, 35.35, 34.22, 33.77.

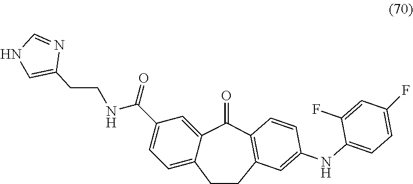 [SMILES: O=C(NCCc1c[nH]cn1)c1ccc2c(c1)C(=O)c1ccc(Nc3ccc(F)cc3F)cc1CC2]

C₂₇H₂₂F₂N₄O₂ (M=472.17 g/mol)

Masse (ESI_{neg}) (m/z): 471.5 [M−H]⁻

IR (ATR) [cm⁻¹] 2921, 2851, 1603, 1579, 1505, 1354, 1258, 1094, 964, 801, 621

¹H-NMR (400 MHz, DMSO-d6) δ 8.68 (t, 1H), 8.60 (s, 1H), 8.32 (s, 1H), 7.99 (d, J=8.8 Hz, 1H), 7.90 (d, J=7.8 Hz, 1H), 7.57 (s, 5H), 7.38 (dd, J=17.6, 8.6 Hz, 4H), 7.11 (t, J=8.0 Hz, 1H), 6.83 (s, 1H), 6.76 (d, J=8.7 Hz, 1H), 6.62 (s, 1H), 3.47 (s, 2H), 3.12 (d, J=8.9 Hz, 2H), 3.05 (d, J=8.1 Hz, 2H), 2.75 (dd, J=13.7, 6.6 Hz, 2H).

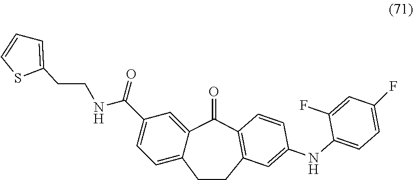 [SMILES: O=C(NCCc1cccs1)c1ccc2c(c1)C(=O)c1ccc(Nc3ccc(F)cc3F)cc1CC2]

C₂₈H₂₂F₂N₂O₂S (M=488.14 g/mol)

Masse (ESI_{neg}) (m/z): 486.9 [M−H]⁻

IR (ATR) [cm⁻¹] 1602, 1580, 1504, 1354, 1258, 1139, 964, 845, 696, 594

¹H-NMR (400 MHz, DMSO-d6) δ 8.75 (s, 1H), 8.60 (s, 1H), 8.34 (s, 1H), 7.99 (dd, J=8.8, 2.2 Hz, 1H), 7.94-7.88 (m, 1H), 7.53 (s, 1H), 7.40 (dd, J=14.2, 5.9 Hz, 4H), 7.36-7.31 (m, 2H), 7.27 (s, 1H), 7.11 (s, 1H), 6.95 (dd, J=5.2, 2.2 Hz, 1H), 6.91 (s, 1H), 6.76 (d, J=8.8 Hz, 1H), 6.62 (s, 1H), 5.39 (s, 1H), 4.59-4.51 (m, 2H), 3.50 (d, J=2.1 Hz, 4H), 3.13-3.05 (m, 4H).

¹³C-NMR (100 MHz, DMSO-d6) δ 190.42, 165.51, 149.31, 145.23, 144.60, 141.55, 139.49, 138.88, 133.32, 130.34, 129.10, 128.90, 128.68, 128.23, 128.05, 126.99, 126.87, 126.27, 125.09, 123.98, 113.51, 112.28, 40.95, 35.35, 33.79, 29.11.

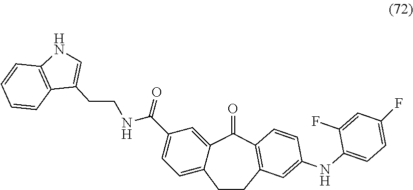 [SMILES: O=C(NCCc1c[nH]c2ccccc12)c1ccc2c(c1)C(=O)c1ccc(Nc3ccc(F)cc3F)cc1CC2]

C₃₂H₂₅F₂N₃O₂ (M=521.19 g/mol)

Masse (ESI_{neg}) (m/z): 520.7 [M−H]⁻

IR (ATR) [cm⁻¹] 2916, 2850, 1633, 1567, 14886, 1259, 1092, 964, 741, 461 ¹H-NMR (400 MHz, DMSO-d6) δ 10.80 (s, 1H), 8.73 (s, 1H), 8.60 (s, 1H), 8.35 (s, 1H), 8.00 (d, J=8.7 Hz, 1H), 7.96-7.84 (m, 2H), 7.58 (d, J=7.7 Hz, 1H), 7.46-7.31 (m, 4H), 7.17 (s, 1H), 7.13-7.03 (m, 2H), 6.98 (t, J=7.3 Hz, 1H), 6.76 (d, J=8.5 Hz, 1H), 6.62 (s, 1H), 3.54 (d, J=6.2 Hz, 2H), 3.11 (s, 2H), 3.06 (s, 2H), 2.95 (t, J=7.2 Hz, 2H).

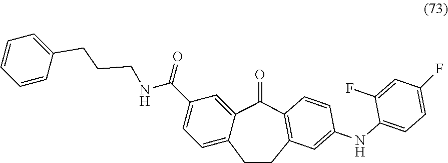 [SMILES: O=C(NCCCc1ccccc1)c1ccc2c(c1)C(=O)c1ccc(Nc3ccc(F)cc3F)cc1CC2]

C₃₁H₂₆F₂N₂O₂ (M=496.20 g/mol)

Masse (ESI_{neg}) (m/z): 495.9 [M−H]⁻

IR (ATR) [cm⁻¹] 3315, 1581, 1603, 1527, 1504, 1262, 1141, 964, 854, 700, 631

¹H-NMR (400 MHz, DMSO-d6) δ 8.59 (s, 2H), 8.34 (s, 1H), 7.99 (s, 1H), 7.91 (s, 1H), 7.41 (s, 3H), 7.23 (s, 4H), 7.17 (t, J=6.9 Hz, 1H), 7.10 (t, J=8.2 Hz, 1H), 6.75 (s, 1H), 6.62 (s, 1H), 3.28 (d, J=6.1 Hz, 2H), 3.10 (s, 2H), 3.05 (s, 2H), 2.62 (s, 2H), 1.83 (sa, 2H).

¹³C-NMR (100 MHz, DMSO-d6) δ 190.49, 165.51, 149.30, 145.21, 144.45, 141.74, 138.86, 133.31, 132.97, 130.37, 129.12, 128.82, 128.26, 128.23, 127.18, 126.26, 125.67, 113.53, 112.30, 111.93, 111.71, 105.16, 104.93, 104.66, 38.90, 34.96, 33.69, 32.41, 30.31.

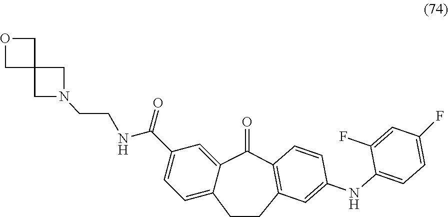 [SMILES: O=C(NCCN1CC2(COC2)C1)c1ccc2c(c1)C(=O)c1ccc(Nc3ccc(F)cc3F)cc1CC2]

C₂₉H₂₇F₂N₃O₃ (M=503.20 g/mol)

Masse (ESI_{neg}) (m/z): 502.1 [M−H]⁻

IR (ATR) [cm⁻¹] 3291, 2924, 2854, 1634, 1603, 1580, 1505, 1355, 1259, 1093, 963, 761, 520

¹H-NMR (400 MHz, CDCl₃) δ 8.31 (d, J=1.3 Hz, 1H), 8.16 (d, J=8.7 Hz, 1H), 7.97 (dd, J=7.8, 1.5 Hz, 1H), 7.39-7.32 (m, 1H), 7.29 (d, J=7.9 Hz, 1H), 7.26 (s, 1H), 6.98-6.83 (m, 2H), 6.76 (s, 1H), 6.66 (d, J=1.8 Hz, 1H), 5.96 (s, 1H), 4.74 (s, 4H), 3.42 (d, J=5.7 Hz, 4H), 3.21-3.14 (m, 2H), 3.13-3.06 (m, 2H), 2.62 (t, J=5.6 Hz, 2H), 2.17 (s, 2H).

¹³C-NMR (100 MHz, CDCl₃) δ 191.45, 166.70, 148.05, 145.33, 145.09, 138.92, 134.19, 132.98, 131.40, 129.39, 129.26, 128.56, 123.97, 123.88, 114.61, 113.30, 111.54, 111.28, 105.06, 104.82, 104.80, 104.56, 81.20, 63.71, 57.87, 39.18, 37.67, 35.84, 34.71.

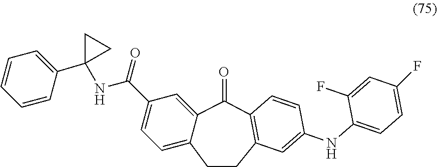 [SMILES: O=C(NC1(c2ccccc2)CC1)c1ccc2c(c1)C(=O)c1ccc(Nc3ccc(F)cc3F)cc1CC2]

C₃₁H₂₄F₂N₂O₂ (M=494.18 g/mol)

Masse (ESI_{neg}) (m/z): 493.0 [M−H]⁻

IR (ATR) [cm⁻¹] 3268, 2922, 2850, 1632, 1603, 1581, 1505, 1353, 1257, 1139, 964, 844, 598

¹H-NMR (400 MHz, CDCl₃) δ 8.34 (s, 1H), 8.11 (d, J=8.6 Hz, 1H), 7.98 (d, J=7.4 Hz, 1H), 7.36-7.29 (m, 2H), 7.15 (d, J=8.2 Hz, 2H), 6.90 (dd, J=17.2, 7.7 Hz, 2H), 6.79 (d, J=8.5 Hz, 1H), 6.63 (s, 1H), 6.00 (s, 1H), 3.13 (s, 2H), 3.07 (s, 2H), 1.35 (s, 2H), 1.33 (s, 2H).

¹³C-NMR (100 MHz, CDCl₃) δ 191.42, 166.66, 148.15, 145.37, 145.32, 142.28, 138.80, 134.22, 132.91, 131.63, 129.55, 129.16, 128.36, 128.33, 126.40, 125.69, 124.08, 123.99, 114.57, 113.27, 111.49, 111.31, 105.05, 104.81, 104.55, 35.81, 35.42, 34.73, 17.78.

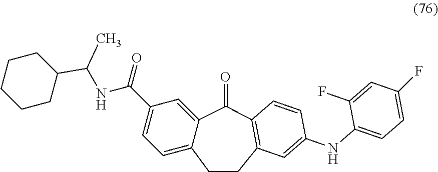 [SMILES: CC(NC(=O)c1ccc2c(c1)C(=O)c1ccc(Nc3ccc(F)cc3F)cc1CC2)C1CCCCC1]

C₃₀H₃₀F₂N₂O₂ (M=488.23 g/mol)

Masse (ESI_{neg}) (m/z): 487.1 [M−H]⁻

IR (ATR) [cm⁻¹] 3268, 2922, 2850, 2361, 1633, 1603, 1581, 1505, 1257, 1139, 1094, 964, 598

¹H-NMR (400 MHz, CDCl₃) δ 8.24 (s, 1H), 8.11 (d, J=8.2 Hz, 1H), 7.94 (d, J=6.6 Hz, 1H), 7.31 (d, J=5.8 Hz, 1H), 6.87 (d, J=8.5 Hz, 2H), 6.80 (d, J=8.7 Hz, 1H), 6.62 (s, 1H), 6.13 (d, J=8.1 Hz, 1H), 6.07 (s, 1H), 4.04 (s, 1H), 3.12 (s, 2H), 3.06 (s, 2H), 2.76 (s, 1H), 1.71 (d, J=10.0 Hz, 4H), 1.26-1.06 (m, 8H), 1.03-0.94 (m, 2H).

¹³C-NMR (100 MHz, CDCl₃) δ 191.51, 165.87, 160.01, 157.67, 156.56, 154.10, 148.17, 145.35, 144.93, 138.86, 134.17, 133.49, 131.51, 129.41, 128.04, 124.82, 124.07, 114.60, 113.24, 111.51, 105.03, 104.77, 104.54, 50.03, 43.27, 38.58, 35.86, 34.68, 29.28, 29.15, 26.37, 26.16, 18.00.

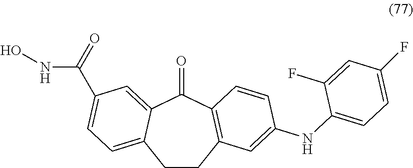 [SMILES: O=C(NO)c1ccc2c(c1)C(=O)c1ccc(Nc3ccc(F)cc3F)cc1CC2]

C₂₂H₁₆F₂N₂O₃ (M=394.11 g/mol)

Masse (ESI_{neg}) (m/z): 392.5 [M−H]⁻

IR (ATR) [cm⁻¹] 2920, 2851, 1602, 1505, 1354, 1258, 1093, 1028, 839, 596, 451

¹H-NMR (400 MHz, DMSO-d6) δ 11.30 (s, 1H), 9.07 (s, 1H), 8.60 (s, 1H), 8.25 (s, 1H), 7.99 (d, J=8.7 Hz, 1H), 7.82 (d, J=7.7 Hz, 1H), 7.40 (td, J=15.1, 8.9 Hz, 4H), 7.11 (t, J=7.8 Hz, 1H), 6.76 (d, J=8.4 Hz, 1H), 6.62 (s, 1H), 3.11 (d, J=8.8 Hz, 2H), 3.05 (s, 2H).

¹³C-NMR (100 MHz, DMSO-d6) δ 190.74, 164.11, 160.44, 158.23, 154.61, 149.83, 145.74, 145.15, 139.37, 133.88, 131.86, 130.45, 129.52, 127.61, 126.78, 125.74, 113.99, 112.79, 112.44, 112.22, 105.67, 105.44, 105.17, 35.83, 34.31.

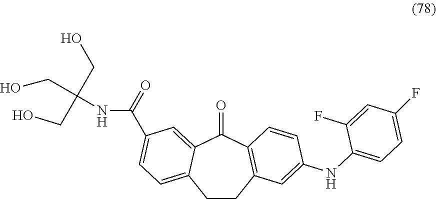 [SMILES: O=C(NC(CO)(CO)CO)c1ccc2c(c1)C(=O)c1ccc(Nc3ccc(F)cc3F)cc1CC2]

C₂₆H₂₄F₂N₂O₅ (M=482.17 g/mol)

Masse (ESI_{neg}) (m/z): 480.9 [M−H]⁻

IR (ATR) [cm⁻¹] 3300, 1602, 1580, 1505, 1403, 1354, 1258, 1214, 1042, 846, 595

¹H-NMR (400 MHz, DMSO-d6) δ 8.60 (s, 1H), 8.27 (d, J=1.5 Hz, 1H), 8.00 (d, J=8.8 Hz, 1H), 7.88 (dd, J=7.8, 1.6 Hz, 1H), 7.47-7.33 (m, 4H), 7.10 (dd, J=11.6, 4.9 Hz, 1H), 6.76 (d, J=8.8 Hz, 1H), 6.62 (s, 1H), 4.77 (t, J=5.7 Hz, 3H), 3.70 (d, J=5.6 Hz, 6H), 3.14-3.07 (m, 2H), 3.08-2.94 (m, 2H).

¹³C-NMR (100 MHz, DMSO-d6) δ 190.39, 166.60, 149.33, 145.25, 144.57, 138.75, 133.52, 133.36, 130.55, 129.21, 128.74, 127.10, 126.28, 126.18, 113.51, 112.29, 111.94, 111.69, 105.18, 104.92, 69.77, 60.46, 35.43, 33.77.

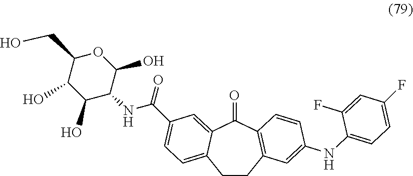 [SMILES: O=C(N[C@@H]1[C@@H](O)[C@H](O)[C@@H](CO)O[C@H]1O)c1ccc2c(c1)C(=O)c1ccc(Nc3ccc(F)cc3F)cc1CC2]

C₂₈H₂₆F₂N₂O₄ (M=540.17 g/mol)

Masse (ESI_{neg}) (m/z): 538.7 [M−H]⁻

IR (ATR) [cm⁻¹] 3300, 1633, 1603, 1505, 1355, 1259, 1094, 1027, 965, 845, 516

¹H-NMR (400 MHz, DMSO-d6) δ 8.59 (s, 1H), 8.37 (s, 1H), 8.20 (d, J=7.1 Hz, 1H), 8.04-7.90 (m, 3H), 7.40 (dd, J=16.2, 7.8 Hz, 3H), 7.11 (s, 1H), 6.76 (d, J=8.8 Hz, 1H), 6.63 (s, 1H), 6.48 (d, J=4.2 Hz, 1H), 5.09 (s, 1H), 4.99 (s, 1H), 4.72 (d, J=4.6 Hz, 1H), 4.48 (t, J=5.4 Hz, 1H), 3.83-3.74 (m, 2H), 3.69-3.60 (m, 2H), 3.24-3.17 (m, 1H), 3.12 (d, J=6.0 Hz, 2H), 3.05 (s, 2H).

¹³C-NMR (100 MHz, DMSO-d6) δ 191.51, 166.75, 163.23, 150.21, 146.14, 145.40, 139.78, 134.19, 133.75, 131.57, 130.28, 129.60, 128.10, 127.18, 114.48, 113.21, 112.85, 112.63, 105.84, 91.33, 73.02, 71.94, 70.92, 62.09, 56.35, 36.66, 36.33, 34.68, 31.67.

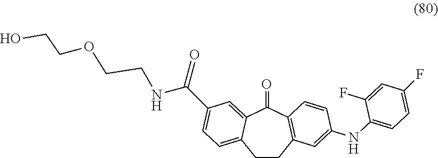 [SMILES: O=C(NCCOCCO)c1ccc2c(c1)C(=O)c1ccc(Nc3ccc(F)cc3F)cc1CC2]

C₂₆H₂₄F₂N₂O₄ (M=466.17 g/mol)

Masse (ESI_{neg}) (m/z): 464.9 [M−H]⁻

IR (ATR) [cm⁻¹] 2950, 1651, 1601, 1578, 1506, 1348, 1261, 1211, 1133, 1095, 966, 844, 784

¹H-NMR (400 MHz, DMSO-d6) δ 8.67-8.57 (m, 2H), 8.34 (d, J=1.5 Hz, 1H), 8.00 (d, J=8.8 Hz, 1H), 7.92 (dd, J=7.9, 1.6 Hz, 1H), 7.45-7.33 (m, 3H), 7.14-7.05 (m, 1H), 6.76 (d, J=8.9 Hz, 1H), 6.63 (s, 1H), 4.63 (t, J=5.3 Hz, 1H), 3.54 (t, J=6.0 Hz, 2H), 3.51-3.47 (m, 2H), 3.45 (d, J=4.7 Hz, 2H), 3.43 (s, 2H), 3.11 (d, J=9.1 Hz, 2H), 3.05 (s, 2H), 2.68 (s, 1H).

¹³C-NMR (100 MHz, DMSO-d6) δ 190.44, 165.66, 159.75, 159.63, 157.33, 157.22, 156.97, 156.84, 154.50, 149.30, 145.25, 144.60, 138.86, 133.34, 132.71, 130.37, 129.19, 128.87, 127.13, 126.25, 124.86, 113.52, 112.28, 111.94, 111.90, 105.17, 104.93, 72.06, 68.81, 60.19, 38.19, 35.36, 33.77.

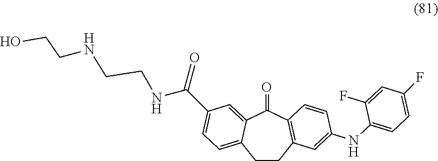 [SMILES: O=C(NCCNCCO)c1ccc2c(c1)C(=O)c1ccc(Nc3ccc(F)cc3F)cc1CC2]

C₂₆H₂₅F₂N₃O₃ (M=465.19 g/mol)

Masse (ESI_{neg}) (m/z): 463.9 [M−H]⁻

IR (ATR) [cm⁻¹] 1601, 1557, 1524, 1439, 1354, 1257, 1140, 848, 754, 687

¹H-NMR (400 MHz, DMSO-d6) δ 8.61 (s, 1H), 8.57 (t, J=5.3 Hz, 1H), 8.34 (d, J=1.3 Hz, 1H), 8.00 (d, J=8.8 Hz, 1H), 7.92 (dd, J=7.8, 1.7 Hz, 1H), 7.48-7.32 (m, 3H), 7.11 (d, J=1.9 Hz, 1H), 6.76 (dd, J=8.8, 1.4 Hz, 1H), 6.63 (s, 1H), 4.55 (s, 1H), 3.46 (t, J=5.7 Hz, 2H), 3.37 (s, 2H), 3.12 (d, J=9.3 Hz, 2H), 3.04 (d, J=8.8 Hz, 2H), 2.73 (t, J=6.4 Hz, 2H), 2.64 (t, J=5.7 Hz, 2H), 2.08 (s, 1H).

¹³C-NMR (100 MHz, DMSO-d6) δ 190.46, 165.63, 159.64, 157.34, 157.22, 156.98, 156.86, 154.52, 149.31, 145.23, 144.52, 138.85, 133.32, 132.85, 130.40, 129.16, 128.83, 127.15, 126.26, 124.88, 124.76, 113.52, 112.29, 111.93, 1105.17, 104.93, 104.67, 60.05, 51.21, 48.32, 35.38,

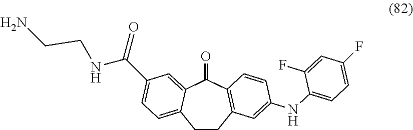 [SMILES: NCCNC(=O)c1ccc2c(c1)C(=O)c1ccc(Nc3ccc(F)cc3F)cc1CC2]

C₂₄H₂₁F₂N₃O₂ (M=421.16 g/mol)

Masse (ESI_{neg}) (m/z): 420.0 [M−H]⁻

IR (ATR) [cm⁻¹] 1602, 1577, 1504, 1354, 1258, 1214, 1114, 1094, 964, 844, 516

¹H-NMR (400 MHz, DMSO-d6) δ 8.61 (s, 1H), 8.55 (d, J=5.0 Hz, 1H), 8.34 (s, 1H), 8.00 (d, J=8.8 Hz, 1H), 7.93 (d, J=7.7 Hz, 1H), 7.48-7.36 (m, 3H), 7.11 (t, J=7.5 Hz, 1H), 6.76 (d, J=8.5 Hz, 1H), 6.63 (s, 1H), 3.50 (s, 2H), 3.17 (s, 2H), 3.11 (d, J=6.0 Hz, 2H), 3.05 (s, 2H), 2.69 (t, J=6.2 Hz, 2H).

¹³C-NMR (100 MHz, DMSO-d6) δ 190.48, 165.68, 149.30, 145.22, 144.46, 138.84, 133.31, 132.94, 130.41, 129.17, 128.80, 127.17, 126.26, 124.86, 113.52, 112.29, 111.94, 111.72, 105.18, 104.94, 104.67, 42.75, 41.11, 35.38, 33.77.

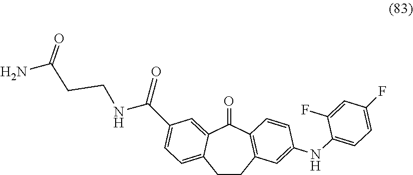 [SMILES: NC(=O)CCNC(=O)c1ccc2c(c1)C(=O)c1ccc(Nc3ccc(F)cc3F)cc1CC2]

C₂₅H₂₁F₂N₃O₃ (M=449.16 g/mol)

Masse (ESI_{neg}) (m/z): 448.2 [M−H]⁻

IR (ATR) [cm⁻¹] 1654, 1602, 1544, 1504, 1361, 1258, 1113, 968, 847, 569, 470

¹H-NMR (400 MHz, DMSO-d6) (400 MHz, DMSO) δ 8.68-8.58 (m, 2H), 8.32 (s, 2H), 7.99 (d, J=8.8 Hz, 1H), 7.90 (d, J=7.7 Hz, 1H), 7.48-7.32 (m, 4H), 7.11 (t, J=8.2 Hz, 1H), 6.84 (s, 1H), 6.76 (d, J=8.7 Hz, 1H), 6.62 (s, 1H), 3.51 (s, 2H), 3.11 (d, J=8.8 Hz, 2H), 3.05 (s, 2H), 2.36 (t, J=7.1 Hz, 2H), 1.23 (s, 2H).

¹³C-NMR (100 MHz, DMSO-d6) δ 190.46, 172.59, 165.52, 149.31, 145.22, 144.55, 138.84, 133.31, 132.79, 130.31, 129.12, 128.86, 127.17, 126.14, 125.06, 124.84, 113.53, 112.28, 106.98, 104.92, 36.01, 35.35, 34.92, 33.78.

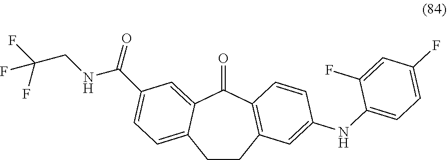 [SMILES: O=C(NCC(F)(F)F)c1ccc2c(c1)C(=O)c1ccc(Nc3ccc(F)cc3F)cc1CC2]

C₂₄H₁₇F₅N₂O₂ (M=460.12 g/mol)

Masse (ESI_{neg}) (m/z): 459.8 [M−H]⁻

IR (ATR) [cm⁻¹] 1668, 1604, 1539, 1355, 1243, 1151, 964, 833, 729

¹H-NMR (400 MHz, DMSO-d6) δ 9.22 (s, 1H), 8.60 (s, 1H), 8.39 (s, 1H), 7.97 (s, 2H), 7.44 (s, 3H), 7.10 (s, 1H), 6.78 (s, 1H), 6.63 (s, 1H), 4.07 (s, 2H), 3.12 (s, 2H), 3.06 (s, 2H).

¹³C-NMR (100 MHz, DMSO-d6) δ 190.60, 166.25, 159.78, 159.67, 157.40, 157.25, 157.00, 156.88, 154.54, 149.39, 145.37, 145.25, 138.99, 133.36, 131.52, 130.61, 129.42, 129.12, 127.05, 126.30, 126.17, 124.85, 124.73, 123.39, 113.51, 112.31, 111.89, 111.71, 105.16, 104.91, 104.65, 40.32, 39.98, 35.28, 33.81.

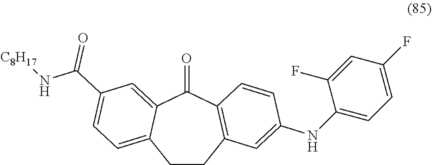

C₃₀H₃₂F₂N₂O₂ (M=490.24 g/mol)

Masse (ESI_{neg}) (m/z): 489.9 [M−H]⁻

IR (ATR) [cm⁻¹] 3274, 2923, 2853, 1603, 1505, 1257, 1139, 1094, 964, 845, 597

¹H-NMR (400 MHz, DMSO-d6) δ 8.64-8.48 (m, 2H), 8.33 (d, J=0.5 Hz, 1H), 8.01 (s, 1H), 7.90 (s, 1H), 7.39 (s, 3H), 7.10 (s, 1H), 6.77 (s, 1H), 6.63 (s, 1H), 3.24 (s, 2H), 3.10 (s, 2H), 3.05 (s, 2H), 1.50 (s, 2H), 1.23 (s, 11H), 0.83 (s, 3H).

¹³C-NMR (100 MHz, DMSO-d6) δ 190.39, 165.41, 159.63, 156.97, 154.51, 149.22, 145.18, 144.38, 138.94, 133.29, 128.79, 126.11, 124.90, 113.63, 112.05, 105.20, 38.88, 35.38, 33.66, 31.06, 28.69, 26.44, 22.01, 13.84.

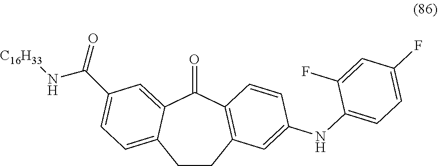

C₃₈H₄₈F₂N₂O₂ (M=602.37 g/mol)

Masse (ESI_{neg}) (m/z): 602.1 [M−H]⁻

IR (ATR) [cm⁻¹] 3345, 2919, 2847, 1631, 1604, 1495, 1358, 1265, 963, 858, 624

¹H-NMR (400 MHz, DMSO-d6) δ 9.77 (s, 1H), 7.79 (s, 1H), 7.42 (s, 1H), 7.31 (s, 1H), 6.91 (t, J=9.8 Hz, 1H), 6.76 (s, 1H), 6.41 (s, 1H), 5.01 (s, 2H), 3.36 (s, 26H), 3.08 (s, 2H), 2.24 (d, J=5.4 Hz, 4H), 1.18 (s, 1H).

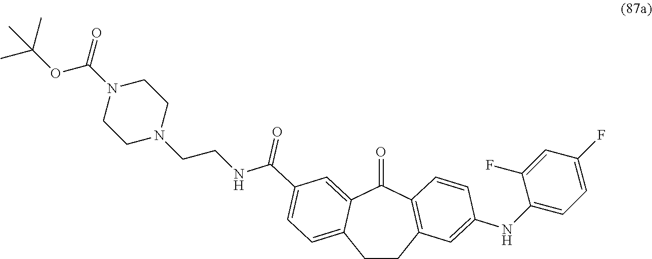 [SMILES: CC(C)(C)OC(=O)N1CCN(CCNC(=O)c2ccc3c(c2)C(=O)c2ccc(Nc4ccc(F)cc4F)cc2CC3)CC1]

C₃₃H₃₆F₂N₄O₄ (M=590.27 g/mol)

Masse (ESI_{neg}) (m/z): 598.8 [M−H]⁻

IR (ATR) [cm⁻¹] 1634, 1581, 1505, 1354, 1259, 1165, 1002, 964, 845, 517

¹H-NMR (400 MHz, DMSO-d6) δ 8.60 (s, 1H), 8.54 (s, 1H), 8.32 (s, 1H), 8.00 (d, J=8.7 Hz, 1H), 7.90 (d, J=7.7 Hz, 1H), 7.49-7.31 (m, 3H), 7.11 (t, J=7.8 Hz, 1H), 6.76 (d, J=8.7 Hz, 1H), 6.62 (s, 1H), 3.30 (s, 4H), 3.11 (d, J=8.3 Hz, 2H), 3.05 (s, 2H), 2.38 (s, 2H), 1.38 (s, 9H).

¹³C-NMR (100 MHz, DMSO-d6) δ 190.43, 165.48, 153.80, 149.32, 145.22, 144.54, 138.86, 133.32, 132.83, 130.34, 129.09, 128.88, 127.16, 126.27, 113.52, 112.29, 111.93, 111.71, 105.16, 104.92, 104.66, 78.69, 56.79, 52.43, 36.71, 35.36, 33.78, 28.03.

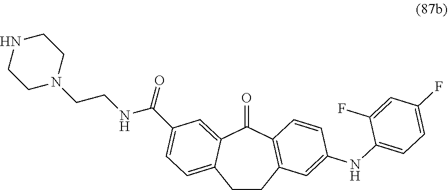 [SMILES: O=C(NCCN1CCNCC1)c1ccc2c(c1)C(=O)c1ccc(Nc3ccc(F)cc3F)cc1CC2]

C₂₈H₂₈F₂N₄O₂ (M=490.22 g/mol)

Masse (ESIₚₒₛ) (m/z): 491.9 [M+H]⁺

IR (ATR) [cm⁻¹] 1632, 1603, 1577, 1584, 1354, 1258, 1139, 964, 844, 729

¹H-NMR (400 MHz, DMSO-d6) δ 8.61 (s, 1H), 8.27 (s, 1H), 7.98 (t, J=13.8 Hz, 1H), 7.87 (d, J=7.1 Hz, 1H), 7.40 (d, J=8.3 Hz, 4H), 7.09 (d, J=20.9 Hz, 1H), 6.76 (d, J=7.8 Hz, 1H), 6.59 (d, J=24.7 Hz, 1H), 4.77 (s, 4H), 3.69 (s, 8H), 3.07 (d, J=31.4 Hz, 4H).

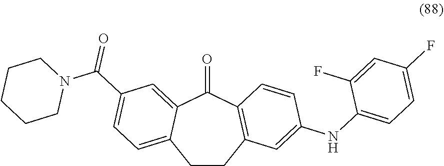 [SMILES: O=C1c2ccc(Nc3ccc(F)cc3F)cc2CCc2ccc(C(=O)N3CCCCC3)cc21]

C₂₇H₂₄F₂N₂O₂ (M=446.18 g/mol)

Masse (ESI_{neg}) (m/z): 445.1 [M−H]⁻

IR (ATR) [cm⁻¹] 2922, 2852, 2359, 2341, 1602, 1579, 1506, 1437, 1353, 1255, 1093, 799

¹H-NMR (400 MHz, DMSO-d6) δ 8.60 (s, 1H), 7.98 (d, J=8.8 Hz, 1H), 7.82 (s, 1H), 7.49-7.33 (m, 5H), 7.10 (t, J=7.5 Hz, 1H), 6.75 (d, J=8.5 Hz, 1H), 6.62 (s, 1H), 3.61 (s, 4H), 3.08 (d, J=6.9 Hz, 2H), 3.06 (s, 2H), 1.64-1.39 (m, 7H), 1.31-1.12 (m, 2H).

¹³C-NMR (100 MHz, DMSO-d6) δ 190.07, 168.25, 149.33, 145.34, 142.93, 138.63, 134.56, 133.46, 130.05, 129.02, 128.54, 127.09, 113.45, 112.24, 35.37, 33.82, 23.96.

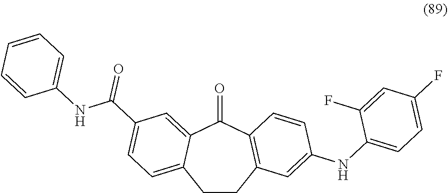 [SMILES: O=C(Nc1ccccc1)c1ccc2c(c1)C(=O)c1ccc(Nc3ccc(F)cc3F)cc1CC2]

C₂₈H₂₀F₂N₂O₂ (M=454.15 g/mol)

Masse (ESI_{neg}) (m/z): 453.0 [M−H]⁻

¹H-NMR (400 MHz, DMSO-d6) δ 10.35 (s, 1H), 8.62 (s, 1H), 8.44 (d, J=1.6 Hz, 1H), 8.07-8.00 (m, 2H), 7.77 (d, J=7.8 Hz, 2H), 7.53-7.32 (m, 5H), 7.11 (dd, J=10.1, 4.3 Hz, 2H), 6.77 (d, J=8.8 Hz, 1H), 6.64 (s, 1H), 3.18-3.11 (m, 2H), 3.07 (d, J=8.8 Hz, 2H).

¹³C-NMR (100 MHz, DMSO-d6) δ 190.35, 164.82, 149.37, 145.26, 145.07, 139.05, 138.94, 133.39, 130.81, 129.61, 129.00, 128.55, 127.10, 123.68, 120.43, 113.51, 112.30, 35.35, 33.82.

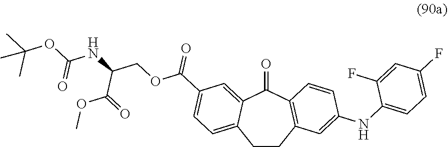 [SMILES: COC(=O)[C@H](COC(=O)c1ccc2c(c1)C(=O)c1ccc(Nc3ccc(F)cc3F)cc1CC2)NC(=O)OC(C)(C)C]

C₃₁H₃₀F₂N₂O₇ (M=580.20 g/mol)

HPLC 9.06 min

Masse (ESI_{neg}) (m/z): 579.4 [M−H]⁻

IR (ATR) [cm⁻¹] 1714, 1690, 1604, 1581, 1505, 1355, 1236, 1158, 840, 783, 760

¹H-NMR (400 MHz, DMSO-d6) δ 8.62 (s, 1H), 8.41 (s, 1H), 8.05-7.96 (m, 2H), 7.61 (d, J=7.3 Hz, 1H), 7.51-7.45 (m, 1H), 7.39 (dd, J=21.5, 9.7 Hz, 2H), 7.11 (t, J=7.5 Hz, 1H), 6.76 (d, J=8.6 Hz, 1H), 6.62 (s, 1H), 4.54 (d, J=7.1 Hz, 2H), 4.44 (dd, J=11.4, 8.4 Hz, 1H), 3.67 (s, 3H), 3.14 (s, 2H), 3.05 (s, 2H), 1.36 (s, 9H).

¹³C-NMR (100 MHz, DMSO-d6) 189.81, 189.71, 170.18, 164.90, 155.39, 149.47, 147.09, 145.28, 139.18, 133.46, 132.20, 131.37, 129.37, 127.77, 126.85, 126.32, 113.45, 112.30, 111.95, 111.73, 105.18, 104.94, 104.68, 78.59, 63.74, 52.47, 52.14, 35.17, 33.96, 28.04.

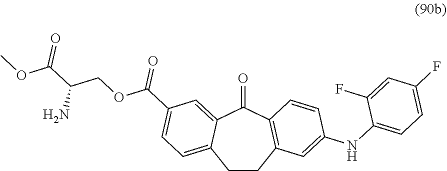 [SMILES: COC(=O)[C@@H](N)COC(=O)c1ccc2c(c1)C(=O)c1ccc(Nc3ccc(F)cc3F)cc1CC2]

C₂₆H₂₂F₂N₂O₅ (M=480.15 g/mol)

Masse (ESI_{neg}) (m/z): 479.2 [M−H]⁻

IR (ATR) [cm⁻¹] 1711, 1602, 1581, 1507, 1353, 1237, 1139, 1110, 963, 846, 761

¹H-NMR (400 MHz, DMSO-d6) δ 8.60 (s, 1H), 8.43 (d, J=1.5 Hz, 1H), 8.02-7.95 (m, 2H), 7.41 (dd, J=8.5, 3.6 Hz, 2H), 7.35 (s, 1H), 7.10 (s, 1H), 6.76 (dd, J=8.8, 1.3 Hz, 1H), 6.62 (s, 1H), 3.82-3.75 (m, 3H), 3.14-3.08 (m, 2H), 3.06-3.01 (m, 2H), 1.89 (d, J=8.6 Hz, 1H), 1.25 (dd, J=21.6, 11.8 Hz, 2H).

¹³C-NMR (100 MHz, DMSO-d6) δ 190.08, 171.99, 167.01, 159.75, 157.34, 156.98, 154.52, 154.39, 149.35, 146.02, 145.22, 138.79, 133.43, 132.22, 131.40, 130.25, 129.19, 127.13, 126.25, 124.89, 113.48, 112.29, 111.91, 105.13, 104.63, 52.57, 51.69, 35.23, 34.01, 21.03.

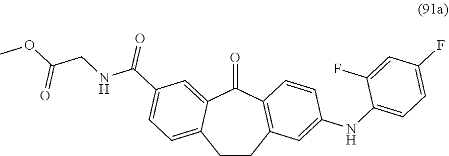 [SMILES: COC(=O)CNC(=O)c1ccc2c(c1)C(=O)c1ccc(Nc3ccc(F)cc3F)cc1CC2]

C₂₅H₂₀F₂N₂O₄ (M=450.14 g/mol)

Masse (ESI_{neg}) (m/z): 448.8 [M−H]⁻

IR (ATR) [cm⁻¹] 3383, 3278, 1758, 1650, 1606, 1575, 1258, 1195, 962, 760, 512

¹H-NMR (400 MHz, CDCl₃) ¹H NMR (400 MHz,) δ 8.22 (s, 1H), 7.95 (d, J=7.5 Hz, 1H), 7.78 (s, 1H), 7.18 (d, J=8.7 Hz, 2H), 6.75 (d, J=6.1 Hz, 2H), 6.66 (d, J=7.7 Hz, 1H), 6.48 (s, 1H), 4.03 (s, 2H), 3.61 (s, 3H), 3.20 (s, 1H), 3.02 (s, 2H), 2.95 (s, 2H).

¹³C-NMR (100 MHz, CDCl₃) δ 191.66, 170.22, 167.37, 148.73, 145.31, 138.72, 133.69, 131.66, 130.79, 128.84, 124.41, 113.76, 112.63, 111.07, 104.35, 77.16, 51.88, 41.20, 35.47, 34.20.

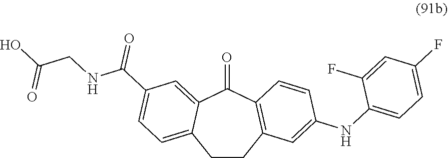 [SMILES: O=C(O)CNC(=O)c1ccc2c(c1)C(=O)c1ccc(Nc3ccc(F)cc3F)cc1CC2]

C₂₄H₁₈F₂N₂O₄ (M=436.12 g/mol)

Masse (ESI_{neg}) (m/z): 434.7 [M−H]⁻

IR (ATR) [cm⁻¹] 3292, 1715, 1601, 1519, 1397, 1284, 1260, 1211, 1138, 964, 841

¹H-NMR (400 MHz, MeOD-d4) δ 8.43 (d, J=1.6 Hz, 1H), 8.07 (d, J=8.8 Hz, 1H), 7.92 (dd, J=7.8, 1.4 Hz, 1H), 7.42-7.35 (m, 2H), 7.12-7.02 (m, 1H), 6.98 (t, J=8.5 Hz, 1H), 6.83-6.73 (m, 1H), 6.65 (s, 1H), 4.10 (s, 2H), 3.23-3.14 (m, 2H), 3.13-3.05 (m, 2H).

¹³C-NMR (100 MHz, MeOD-d4) δ 193.11, 173.15, 169.66, 159.46, 156.53, 151.40, 147.17, 147.13, 140.80, 134.93, 133.66, 131.75, 130.72, 130.25, 128.95, 127.17, 127.08, 126.55, 115.02, 113.71, 112.57, 112.53, 112.35, 112.31, 105.85, 105.61, 105.34, 101.28, 42.40, 37.01, 35.63.

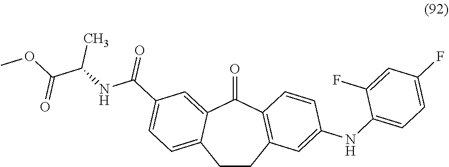 [SMILES: COC(=O)[C@H](C)NC(=O)c1ccc2c(c1)C(=O)c1ccc(Nc3ccc(F)cc3F)cc1CC2]

C₂₆H₂₂F₂N₂O₄ (M=464.15 g/mol)

Masse (ESI_{neg}) (m/z): 463.6 [M−H]⁻

IR (ATR) [cm⁻¹] 3304, 2921, 2852, 1738, 1634, 1603, 1505, 1454, 1354, 1172, 1212, 1140, 845

¹H-NMR (400 MHz, DMSO-d6) δ 8.94 (d, J=6.7 Hz, 1H), 8.61 (s, 1H), 8.39 (s, 1H), 8.01 (d, J=8.8 Hz, 1H), 7.95 (d, J=7.8 Hz, 1H), 7.41 (dd, J=16.3, 7.7 Hz, 3H), 7.12 (d, J=8.3 Hz, 1H), 6.77 (d, J=8.7 Hz, 1H), 6.63 (s, 1H), 4.54-4.44 (m, 1H), 3.65 (s, 3H), 3.13 (d, J=6.2 Hz, 2H), 3.06 (s, 2H), 1.41 (d, J=7.1 Hz, 3H).

¹³C-NMR (100 MHz, DMSO-d6) δ 190.44, 173.08, 165.55, 149.33, 145.24, 144.90, 138.96, 133.31, 131.98, 130.63, 129.40, 128.87, 127.10, 126.28, 124.87, 113.53, 112.30, 111.94, 111.72, 105.18, 104.94, 51.83, 48.25, 35.36, 33.78, 16.67.

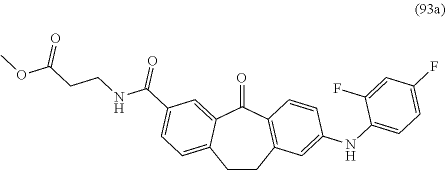 [SMILES: COC(=O)CCNC(=O)c1ccc2c(c1)C(=O)c1ccc(Nc3ccc(F)cc3F)cc1CC2]

C₂₆H₂₂F₂N₂O₄ (M=464.15 g/mol)

Masse (ESI_{neg}) (m/z): 462.8 [M−H]⁻

IR (ATR) [cm⁻¹] 1717, 1602, 1581, 1525, 1434, 1244, 1116, 915, 842, 718, 508

¹H-NMR (400 MHz, DMSO-d6) δ 8.68 (s, 1H), 8.60 (s, 1H), 8.31 (s, 1H), 7.99 (dd, J=8.8, 2.9 Hz, 1H), 7.90 (d, J=7.7 Hz, 1H), 7.46-7.33 (m, 3H), 7.11 (t, J=8.5 Hz, 1H), 6.76 (d, J=8.7 Hz, 1H), 6.62 (s, 1H), 3.60 (d, J=3.0 Hz, 3H), 3.49 (d, J=5.6 Hz, 2H), 3.11 (d, J=7.6 Hz, 2H), 3.04 (d, J=7.7 Hz, 2H), 2.68 (d, J=3.2 Hz, 1H), 2.62-2.55 (m, 2H).

¹³C-NMR (100 MHz, DMSO-d6) δ 190.44, 171.72, 165.63, 149.33, 145.23, 144.65, 138.87, 133.32, 132.60, 130.37, 129.10, 128.89, 127.14, 126.18, 124.84, 113.52, 112.29, 111.94, 111.72, 105.17, 104.93, 104.66, 51.33, 38.20, 35.51, 35.35, 33.77, 33.50.

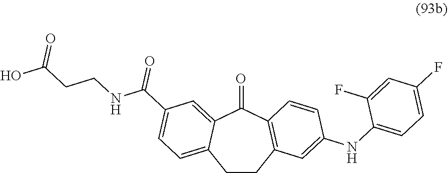 [SMILES: O=C(O)CCNC(=O)c1ccc2c(c1)C(=O)c1ccc(Nc3ccc(F)cc3F)cc1CC2]

C₂₅H₂₀F₂N₂O₄ (M=450.14 g/mol)

Masse (ESI_{neg}) (m/z): 448.8 [M−H]⁻

IR (ATR) [cm⁻¹] 1644, 1601, 1549, 1519, 1188, 1260, 968, 839, 804, 731, 604

¹H-NMR (400 MHz, CDCl₃) δ 12.12 (d, J=12.3 Hz, 1H), 8.65 (t, J=5.3 Hz, 1H), 8.60 (s, 1H), 8.32 (d, J=1.7 Hz, 1H), 7.97 (dd, J=21.3, 7.6 Hz, 1H), 7.90 (dd, J=7.9, 1.8 Hz, 1H), 7.50-7.30 (m, 3H), 7.10 (td, J=8.5, 1.7 Hz, 1H), 6.74 (dt, J=38.6, 19.3 Hz, 1H), 6.64 (d, J=11.1 Hz, 1H), 3.44 (d, J=7.2 Hz, 2H), 3.11 (d, J=9.4 Hz, 2H), 3.04 (d, J=9.0 Hz, 2H), 2.55-2.51 (m, 2H).

¹³C-NMR (100 MHz, CDCl₃) δ 190.69, 173.09, 165.80, 157.21, 154.75, 149.55, 145.50, 144.86, 139.11, 133.57, 132.89, 130.62, 129.36, 129.12, 127.36, 126.52, 126.42, 125.11, 124.98, 113.76, 112.53, 112.19, 111.98, 105.43, 105.19, 104.92, 39.52, 35.83, 35.60, 34.01, 33.94.

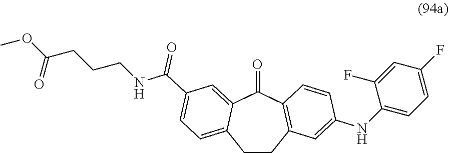 [SMILES: COC(=O)CCCNC(=O)c1ccc2c(c1)C(=O)c1ccc(Nc3ccc(F)cc3F)cc1CC2]

C₂₇H₂₄F₂N₂O₄ (M=478.14 g/mol)

Masse (ESI_{neg}) (m/z): 476.8 [M−H]⁻

IR (ATR) [cm⁻¹] 3290, 1728, 1601, 1505, 1437, 1357, 1261, 1097, 967, 850, 528

¹H-NMR (400 MHz, CDCl₃) δ 8.31 (d, J=1.2 Hz, 1H), 8.10 (d, J=8.7 Hz, 1H), 7.92 (d, J=7.7 Hz, 1H), 7.33 (td, J=8.9, 5.8 Hz, 1H), 6.90 (ddd, J=12.5, 7.7, 3.5 Hz, 2H), 6.81 (dd, J=8.7, 1.8 Hz, 1H), 6.76 (s, 1H), 6.63 (s, 1H), 6.11 (s, 1H), 3.65 (s, 3H), 3.48 (dd, J=12.5, 6.5 Hz, 2H), 3.13 (d, J=9.2 Hz, 2H), 3.09-3.02 (m, 2H), 2.41 (t, J=7.1 Hz, 2H), 2.01-1.88 (m, 2H).

¹³C-NMR (100 MHz, CDCl₃) δ 191.34, 173.87, 166.75, 159.99, 157.66, 156.67, 154.20, 148.11, 145.24, 138.92, 134.13, 133.00, 131.19, 129.38, 129.23, 128.54, 124.83, 123.97, 114.58, 113.25, 111.25, 105.01, 104.75, 104.52, 51.70, 39.59, 35.80, 34.70, 31.61, 24.63.

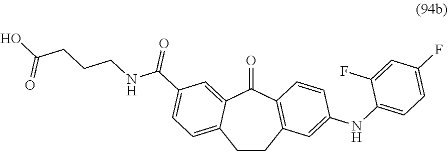 [SMILES: O=C(O)CCCNC(=O)c1ccc2c(c1)C(=O)c1ccc(Nc3ccc(F)cc3F)cc1CC2]

C₂₆H₂₂F₂N₂O₄ (M=464.15 g/mol)

Masse (ESI_{neg}) (m/z): 462.9 [M−H]⁻

IR (ATR) [cm⁻¹] 3281, 1733, 1645, 1601, 1519, 1264, 1096,

¹H-NMR (400 MHz, DMSO-d6) δ 8.45 (s, 1H), 8.16 (d, J=8.7 Hz, 1H), 8.04 (d, J=7.0 Hz, 1H), 7.40-7.30 (m, 3H), 6.98-6.87 (m, 2H), 6.83 (d, J=8.3 Hz, 1H), 6.65 (s, 1H), 6.01 (d, J=31.6 Hz, 1H), 3.54 (d, J=6.0 Hz, 2H), 3.15 (s, 2H), 3.10 (s, 2H), 2.50 (t, J=6.7 Hz, 2H), 2.05-1.96 (m, 2H).

¹³C-NMR (100 MHz, DMSO-d6) δ 192.09, 176.21, 166.92, 148.40, 145.83, 145.32, 138.47, 134.55, 132.82, 132.03, 129.45, 128.71, 124.07, 114.49, 113.26, 111.59, 111.33, 104.83, 104.60, 39.61, 35.86, 34.67, 31.37, 24.57.

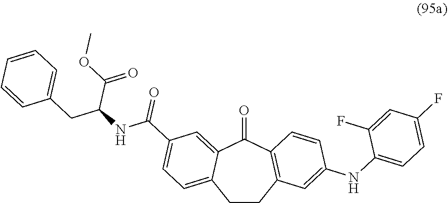 [SMILES: COC(=O)[C@H](Cc1ccccc1)NC(=O)c1ccc2c(c1)C(=O)c1ccc(Nc3ccc(F)cc3F)cc1CC2]

C₃₂H₂₆F₂N₂O₄ (M=540.19 g/mol)

Masse (ESI_{neg}) (m/z): 538.9 [M−H]⁻

IR (ATR) [cm⁻¹] 2157, 2006, 1738, 1651, 1603, 1581, 1258, 1434, 1177, 1118, 1140, 699

¹H-NMR (400 MHz, DMSO-d6) δ 9.00 (d, J=7.5 Hz, 1H), 8.61 (s, 1H), 8.33 (s, 1H), 8.05-7.97 (m, 1H), 7.86 (t, J=9.4 Hz, 1H), 7.47-7.34 (m, 4H), 7.31-7.16 (m, 7H), 7.10 (t, J=7.8 Hz, 1H), 6.76 (d, J=8.7 Hz, 1H), 6.62 (s, 1H), 4.69 (dd, J=13.7, 8.4 Hz, 1H), 3.64 (s, 3H), 3.22-3.16 (m, 1H), 3.16-3.12 (m, 2H), 3.10 (d, J=6.5 Hz, 2H), 3.05 (s, 2H).

¹³C NMR (100 MHz, DMSO-d6) δ 190.87, 172.58, 166.22, 149.85, 145.75, 145.45, 139.45, 138.17, 133.83, 132.42, 131.03, 129.84, 129.47, 129.38, 128.69, 128.48, 127.56, 126.92, 126.75, 126.65, 125.36, 114.03, 112.79, 112.43, 112.21, 105.67, 105.43, 105.16, 54.70, 52.39, 36.65, 35.84, 34.27.

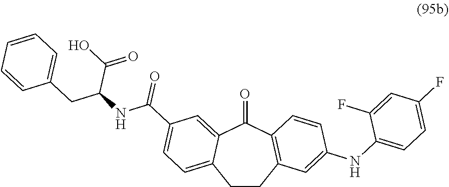 [SMILES: O=C(N[C@@H](Cc1ccccc1)C(=O)O)c1ccc2c(c1)C(=O)c1ccc(Nc3ccc(F)cc3F)cc1CC2]

C₃₁H₂₄F₂N₂O₄ (M=526.17 g/mol)

Masse (ESI_{neg}) (m/z): 525.4 [M−H]⁻

IR (ATR) [cm⁻¹] 1728, 1633, 1602, 1579, 1312, 1407, 1354, 1257, 964, 844, 597

¹H-NMR (400 MHz, DMSO-d6) δ 8.77 (d, J=7.8 Hz, 1H), 8.60 (s, 1H), 8.31 (s, 1H), 8.00 (d, J=8.6 Hz, 1H), 7.85 (s, 1H), 7.39 (d, J=8.1 Hz, 4H), 7.29-7.20 (m, 5H), 6.76 (d, J=9.3 Hz, 1H), 6.63 (s, 1H), 3.50 (s, 1H), 3.18 (s, 1H), 3.08 (s, 4H), 2.97 (s, 2H).

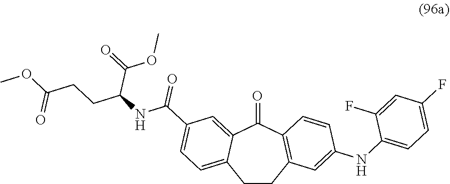 [SMILES: COC(=O)CC[C@H](NC(=O)c1ccc2c(c1)C(=O)c1ccc(Nc3ccc(F)cc3F)cc1CC2)C(=O)OC]

C₂₉H₂₆F₂N₂O₆ (M=536.18 g/mol)

Masse (ESI_{neg}) (m/z): 534.7 [M−H]⁻

IR (ATR) [cm⁻¹] 1732, 1633, 1603, 1505, 1435, 1354, 1258, 1211, 1094, 964, 846

¹H-NMR (400 MHz, DMSO-d6) δ 8.90 (d, J=7.4 Hz, 1H), 8.62 (d, J=7.3 Hz, 1H), 8.37 (d, J=1.8 Hz, 1H), 8.00 (d, J=8.8 Hz, 1H), 7.98-7.91 (m, 1H), 7.47-7.36 (m, 3H), 7.11 (td, J=8.4, 1.7 Hz, 1H), 6.76 (dd, J=8.8, 1.6 Hz, 1H), 6.63 (d, J=5.0 Hz, 1H), 4.47 (ddd, J=9.5, 7.4, 5.4 Hz, 1H), 3.64 (s, 3H), 3.58 (s, 3H), 3.17-3.09 (m, 2H), 3.09-3.01 (m, 2H), 2.68 (s, 1H), 2.45 (t, J=7.5 Hz, 2H), 2.05 (ddd, J=10.3, 9.5, 5.4 Hz, 2H).

¹³C-NMR (100 MHz, DMSO-d6) 190.44, 172.63, 172.13, 165.96, 149.35, 145.26, 145.00, 138.97, 133.32, 130.68, 129.35, 128.89, 127.07, 113.53, 112.29, 51.94, 51.91, 51.32, 35.36, 33.73, 29.90, 25.59.

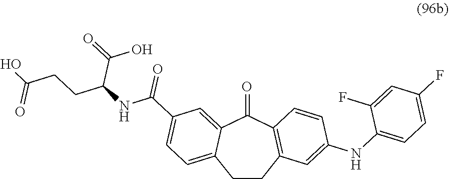 [SMILES: O=C(O)CC[C@H](NC(=O)c1ccc2c(c1)C(=O)c1ccc(Nc3ccc(F)cc3F)cc1CC2)C(=O)O]

C₂₇H₂₂F₂N₂O₆ (M=508.14 g/mol)

Masse (ESI_{neg}) (m/z): 506.9 [M−H]⁻

IR (ATR) [cm⁻¹] 3297, 2923, 1715, 1602, 1506, 1354, 1257, 1139, 1093, 963, 844

¹H-NMR (400 MHz, DMSO-d6) δ 8.64 (s, 1H), 8.51 (d, J=6.9 Hz, 1H), 8.34 (d, J=5.9 Hz, 2H), 8.00 (d, J=8.8 Hz, 1H), 7.94 (d, J=7.7 Hz, 1H), 7.48-7.32 (m, 3H), 7.11 (dd, J=11.9, 5.0 Hz, 1H), 6.76 (d, J=9.1 Hz, 1H), 6.63 (s, 1H), 4.36-4.32 (m, 2H), 3.12 (d, J=8.7 Hz, 2H), 3.04 (d, J=8.0 Hz, 2H), 2.29 (d, J=7.2 Hz, 2H), 1.98 (ddd, J=21.2, 13.1, 7.0 Hz, 2H), 1.25-1.13 (m, 1H).

¹³C-NMR (100 MHz, DMSO-d6) δ 190.44, 174.46, 173.70, 165.21, 159.72, 157.31, 156.95, 154.48, 149.30, 145.27, 144.65, 138.87, 133.35, 132.60, 130.45, 129.22, 127.09, 126.21, 124.87, 113.52, 112.27, 111.90, 105.16, 104.92, 104.66, 52.77, 35.37, 33.78, 31.29, 26.98.

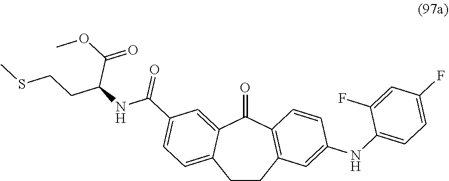 [SMILES: COC(=O)[C@H](CCSC)NC(=O)c1ccc2c(c1)C(=O)c1ccc(Nc3ccc(F)cc3F)cc1CC2]

C₂₈H₂₆F₂N₂O₄S (M=524.16 g/mol)

Masse (ESI_{neg}) (m/z): 523.1 [M−H]⁻

IR (ATR) [cm⁻¹] 2921, 2852, 1737, 1635, 1602, 1505, 1435, 1354, 1257, 1213, 1094, 845

¹H-NMR (400 MHz, DMSO-d6) δ 8.91 (d, J=7.4 Hz, 1H), 8.60 (s, 1H), 8.38 (s, 1H), 8.00 (d, J=8.8 Hz, 1H), 7.96 (d, J=7.8 Hz, 1H), 7.46-7.34 (m, 3H), 7.12 (dd, J=11.0, 4.8 Hz, 1H), 6.76 (d, J=8.8 Hz, 1H), 6.63 (s, 1H), 4.60 (dd, J=14.0, 7.6 Hz, 1H), 3.70-3.63 (m, 3H), 3.13 (d, J=9.4 Hz, 2H), 3.06 (d, J=5.4 Hz, 2H), 2.63-2.53 (m, 2H), 2.08 (t, J=2.5 Hz, 2H), 2.05 (d, J=0.7 Hz, 3H).

¹³C-NMR (100 MHz, DMSO-d6) δ 190.94, 172.85, 166.53, 149.85, 145.77, 145.48, 139.48, 133.82, 132.45, 131.19, 129.87, 129.38, 127.57, 126.79, 114.03, 112.80, 105.68, 105.41, 52.44, 52.18, 35.87, 34.27, 30.56, 30.40, 15.04.

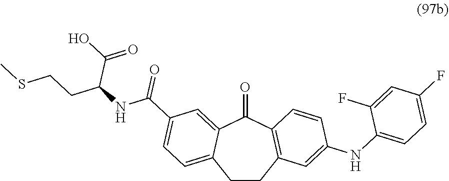 [SMILES: CSCC[C@H](NC(=O)c1ccc2c(c1)C(=O)c1ccc(Nc3ccc(F)cc3F)cc1CC2)C(=O)O]

C₂₇H₂₄F₂N₂O₄S (M=510.14 g/mol)

Masse (ESI_{neg}) (m/z): 509.3 [M−H]⁻

IR (ATR) [cm⁻¹] 1716, 1602, 1506, 1354, 1258, 1115, 1093, 964, 845, 596

¹H-NMR (400 MHz, DMSO-d6) δ 8.37 (d, J=17.4 Hz, 1H), 8.09 (t, J=7.5 Hz, 1H), 7.97-7.87 (m, 1H), 7.32 (d, J=6.2 Hz, 1H), 7.29-7.23 (m, 1H), 6.88 (dd, J=15.9, 7.0 Hz, 2H), 6.80 (d, J=7.7 Hz, 1H), 6.62 (s, 1H), 4.86 (dd, J=13.6, 8.4 Hz, 1H), 3.62 (t, J=23.9 Hz, 1H), 3.28 (s, 3H), 3.10 (d, J=12.2 Hz, 2H), 3.06 (s, 2H), 2.63-2.55 (m, 2H), 2.22 (dd, J=12.9, 7.2 Hz, 1H).

¹³C-NMR (100 MHz, DMSO-d6) δ 191.72, 173.69, 172.55, 167.03, 166.93, 160.15, 157.71, 157.60, 156.78, 156.66, 154.32, 154.20, 148.55, 145.56, 145.49, 139.05, 134.12, 132.19, 132.09, 131.24, 131.17, 129.33, 129.28, 129.06, 129.00, 128.61, 124.36, 124.27, 114.35, 113.12, 111.46, 111.43, 111.24, 111.21, 104.97, 104.73, 104.47, 52.06, 35.78, 34.59, 31.45, 30.18, 15.26.

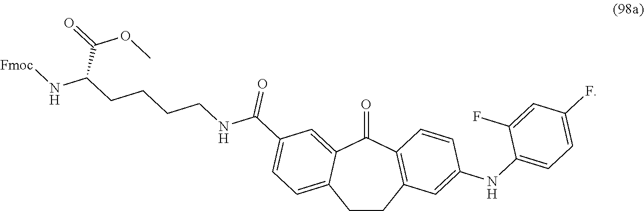

Compound 98a was not further characterized but applied directly to the next step

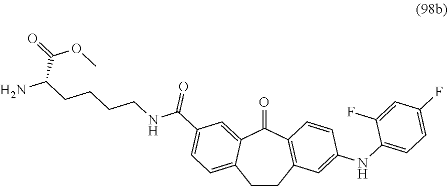 [SMILES: COC(=O)[C@@H](N)CCCCNC(=O)c1ccc2c(c1)C(=O)c1ccc(Nc3ccc(F)cc3F)cc1CC2]

C₂₉H₂₉F₂N₃O₄ (M=521.21 g/mol)

Masse (ESI_{neg}) (m/z): 520.1 [M−H]⁻

IR (ATR) [cm⁻¹] 3272, 2923, 2845, 1731, 1633, 1602, 1501, 1435, 1354, 1258, 1196, 964, 844, 597

¹H-NMR (400 MHz, DMSO-d6) δ 8.62-8.53 (m, 2H), 8.28 (d, J=1.8 Hz, 1H), 7.97 (d, J=8.8 Hz, 1H), 7.88 (dd, J=7.9, 1.8 Hz, 1H), 7.44-7.31 (m, 3H), 7.15-7.06 (m, 1H), 6.74 (d, J=8.9 Hz, 1H), 6.61 (s, 1H), 3.36 (d, J=6.7 Hz, 2H), 3.23 (d, J=6.2 Hz, 3H), 3.09 (s, 2H), 3.04 (s, 2H), 1.49 (dd, J=14.3, 6.7 Hz, 4H), 1.37-1.25 (m, 2H).

¹³C-NMR (100 MHz, DMSO-d6) δ 191.16, 176.16, 166.20, 157.74, 149.82, 145.79, 145.03, 139.34, 133.80, 133.36, 130.81, 129.52, 129.40, 127.61, 126.69, 114.03, 112.73, 112.45, 112.23, 105.61, 105.37, 54.10, 52.00, 40.36, 40.15, 39.94, 39.73, 39.52, 39.31, 39.10, 35.80, 34.24, 29.18, 22.96.

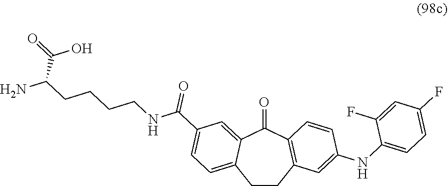 [SMILES: N[C@@H](CCCCNC(=O)c1ccc2c(c1)C(=O)c1ccc(Nc3ccc(F)cc3F)cc1CC2)C(=O)O]

C₂₈H₂₇F₂N₃O₄ (M=507.20 g/mol)

Masse (ESI_{neg}) (m/z): 506.1 [M−H]⁻

IR (ATR) [cm⁻¹] 3296, 2923, 2853, 2777, 1727, 1603, 1557, 1351, 1261, 765 ¹H-NMR (400 MHz, DMSO-d6) δ 8.55 (s, 1H), 8.32 (s, 1H), 8.02 (s, 1H), 7.98 (d, J=8.9 Hz, 1H), 7.87 (t, J=9.6 Hz, 1H), 7.38 (d, J=7.9 Hz, 1H), 7.02 (d, J=7.6 Hz, 1H), 6.95 (t, J=7.6 Hz, 1H), 6.78 (d, J=8.0 Hz, 1H), 6.64 (d, J=8.8 Hz, 1H), 6.59 (t, J=7.5 Hz, 1H), 6.45 (d, J=15.0 Hz, 1H), 4.85 (s, 2H), 3.61 (s, 2H), 3.24 (d, J=5.6 Hz, 3H), 3.09 (d, J=4.2 Hz, 2H), 3.00 (s, 2H), 1.58 (d, J=5.8 Hz, 2H), 1.49 (d, J=7.0 Hz, 6H), 1.39-1.26 (m, 4H).

¹³C-NMR (100 MHz, DMSO-d6) δ 190.46, 176.41, 165.96, 151.41, 145.76, 144.87, 144.13, 139.59, 133.83, 133.61, 129.62, 129.15, 126.60, 126.34, 125.25, 116.94, 115.99, 113.28, 112.31, 54.27, 51.88, 36.18, 34.52, 34.32, 29.31, 25.44, 23.10.

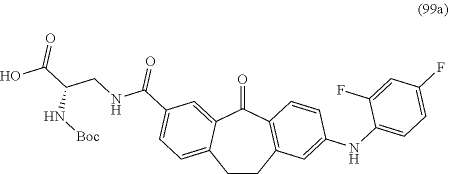 [SMILES: CC(C)(C)OC(=O)N[C@@H](CNC(=O)c1ccc2c(c1)C(=O)c1ccc(Nc3ccc(F)cc3F)cc1CC2)C(=O)O]

Compound 99a was not further characterized but applied directly to the next step

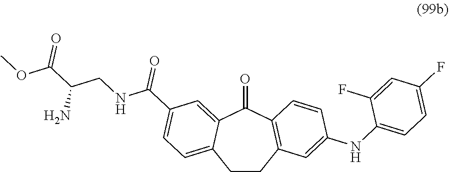 [SMILES: COC(=O)[C@@H](N)CNC(=O)c1ccc2c(c1)C(=O)c1ccc(Nc3ccc(F)cc3F)cc1CC2]

C₂₆H₂₃F₂N₃O₄ (M=479.17 g/mol)

Masse (ESI_{neg}) (m/z): 477.9 [M−H]⁻

IR (ATR) [cm⁻¹] 1732, 1634, 1603, 1580, 1505, 1355, 1260, 1214, 964, 847, 598

¹H-NMR (400 MHz, CDCl₃) δ 8.34 (s, 1H), 8.12 (d, J=8.7 Hz, 1H), 7.94 (d, J=7.8 Hz, 1H), 7.40-7.30 (m, 2H), 7.15 (s, 1H), 6.91 (ddd, J=17.2, 12.7, 6.0 Hz, 3H), 6.83 (d, J=8.6 Hz, 1H), 6.64 (s, 1H), 5.94 (s, 1H), 3.91 (dd, J=13.2, 6.0 Hz, 1H), 3.82 (s, 1H), 3.65 (s, 2H), 3.14 (d, J=9.6 Hz, 2H), 3.08 (s, 2H), 2.17 (s, 3H).

¹³C-NMR (100 MHz, CDCl₃) δ 191.33, 173.64, 166.89, 147.97, 145.28, 145.22, 138.94, 134.22, 132.66, 131.26, 129.33, 128.86, 123.88, 114.58, 113.33, 111.49, 111.31, 105.04, 104.79, 53.96, 52.54, 35.79, 34.68, 30.85.

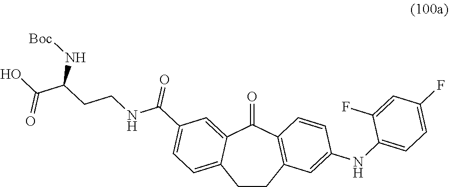 [SMILES: CC(C)(C)OC(=O)N[C@@H](CCNC(=O)c1ccc2c(c1)C(=O)c1ccc(Nc3ccc(F)cc3F)cc1CC2)C(=O)O]

Compound 100a was not further characterized but applied directly to the next step

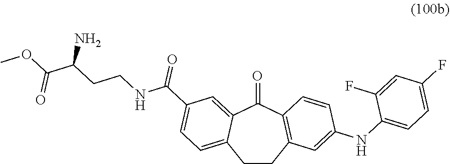 [SMILES: COC(=O)[C@@H](N)CCNC(=O)c1ccc2c(c1)C(=O)c1ccc(Nc3ccc(F)cc3F)cc1CC2]

C₂₇H₂₅F₂N₃O₄ (M=493.18 g/mol)

Masse (ESI_{neg}) (m/z): 491.8 [M−H]⁻

IR (ATR) [cm⁻¹] 1731, 1633, 1603, 1581, 1504, 1355, 1261, 1213. 965, 848, 650

¹H-NMR (400 MHz, CDCl₃) δ 8.36 (d, J=1.3 Hz, 1H), 8.13 (d, J=8.7 Hz, 1H), 8.00-7.91 (m, 2H), 7.39-7.32 (m, 1H), 6.96-6.81 (m, 3H), 6.66 (s, 1H), 5.95 (s, 1H), 3.86-3.77 (m, 1H), 3.71 (s, 3H), 3.57 (dd, J=23.4, 19.1 Hz, 2H), 3.20-3.14 (m, 2H), 3.09 (d, J=9.5 Hz, 2H), 2.67 (d, J=30.0 Hz, 2H), 1.87 (s, 1H).

¹³C-NMR (100 MHz, CDCl₃) δ 191.36, 175.16, 166.49, 147.97, 145.33, 145.04, 138.84, 134.20, 132.96, 131.28, 129.34, 128.77, 124.82, 123.90, 123.78, 114.58, 113.31, 111.53, 111.31, 105.04, 104.80, 104.54, 53.61, 52.34, 37.98, 35.83, 32.75, 30.86.

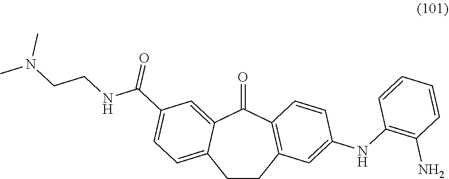 [SMILES: CN(C)CCNC(=O)c1ccc2c(c1)C(=O)c1ccc(Nc3ccccc3N)cc1CC2]

C₂₆H₂₈N₄O₂ (M=428.22 g/mol)

Masse (ESIₚₒₛ) (m/z): 429.1 [M+H]⁺

IR (ATR) [cm⁻¹] 2921, 2852, 1633, 1603, 1574, 1455, 1353, 1262, 1112, 852, 744, 543

¹H-NMR (400 MHz, CDCl₃) δ 8.37 (s, 1H), 8.13 (d, J=8.7 Hz, 1H), 7.96 (d, J=7.6 Hz, 1H), 7.16-7.07 (m, 2H), 6.81 (d, J=21.6 Hz, 2H), 6.65 (dd, J=8.7, 2.0 Hz, 1H), 6.43 (s, 1H), 5.71 (s, 1H), 4.82 (s, 2H) 3.60 (d, J=4.1 Hz, 2H), 3.15-3.09 (m, 4H), 3.08-3.03 (m, 2H), 2.66 (s, 2H), 2.37 (s, 6H).

¹³C-NMR (100 MHz, CDCl₃) δ 191.32, 166.87, 149.95, 145.52, 144.99, 142.82, 139.25, 134.29, 132.91, 131.02, 129.11, 128.93, 128.05, 127.33, 126.85, 125.80, 119.11, 116.37, 113.45, 112.65, 57.94, 44.89, 36.96, 35.94, 34.71.

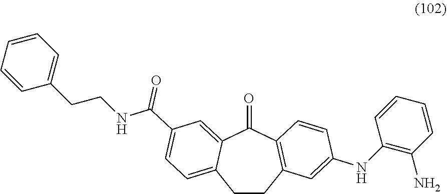 [SMILES: Nc1ccccc1Nc1ccc2c(c1)CCc1ccc(C(=O)NCCc3ccccc3)cc1C2=O]

C₃₀H₂₇N₃O₂ (M=461.21 g/mol)

Masse (ESI_{neg}) (m/z): 460.1 [M−H]⁻

IR (ATR) [cm⁻¹] 3313, 2923, 2854, 1358, 2341, 1633, 1575, 1354, 1261, 1112, 745, 698

¹H-NMR (400 MHz, CDCl₃) δ 8.28 (s, 1H), 8.06 (d, J=8.5 Hz, 1H), 7.88 (d, J=7.4 Hz, 1H), 7.23 (d, J=7.6 Hz, 1H), 7.19-7.00 (m, 4H), 6.95-6.79 (m, 2H), 6.64 (d, J=8.2 Hz, 1H), 6.44 (s, 1H), 6.08 (s, 1H), 3.71 (d, J=2.4 Hz, 2H), 3.07 (s, 2H), 3.00 (s, 2H), 2.65 (t, J=5.7 Hz, 2H).

¹³C-NMR (100 MHz, CDCl₃) δ 190.46, 172.21, 166.14, 151.11, 145.77, 145.09, 139.56, 133.95, 133.05, 130.71, 129.66, 129.24, 126.39, 126.06, 118.69, 117.30, 113.49, 112.48, 36.11, 36.00, 34.33, 34.00.

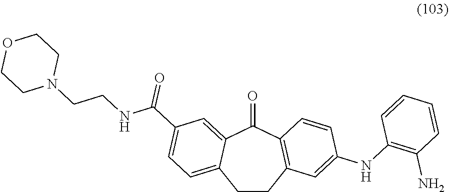 [SMILES: Nc1ccccc1Nc1ccc2c(c1)CCc1ccc(C(=O)NCCN3CCOCC3)cc1C2=O]

C₂₈H₃₀N₃O₃ (M=470.23 g/mol)

Masse (ESIₚₒₛ) (m/z): 471.2 [M+H]+

IR (ATR) [cm⁻¹] 3304, 2921, 2852, 1633, 1602, 1574, 1498, 1353, 1264, 1112, 859, 745, 537

¹H-NMR (400 MHz, DMSO-d6) δ 9.15 (s, 1H), 8.03 (d, J=8.7 Hz, 1H), 7.89 (s, 1H), 7.77 (d, J=8.8 Hz, 1H), 7.65 (d, J=16.5 Hz, 1H), 7.36 (t, J=12.5 Hz, 2H), 6.95 (d, J=8.6 Hz, 1H), 3.89 (s, 3H), 2.98 (d, J=7.5 Hz, 2H), 1.62 (d, J=19.9 Hz, 9H), 1.33 (t, J=7.6 Hz, 3H).

¹³C-NMR (100 MHz, DMSO-d6) δ 169.02, 152.99, 150.04, 148.51, 144.67, 139.11, 133.88, 132.29, 126.99, 125.27, 124.12, 121.81, 118.31, 114.45, 109.29, 84.05, 56.00, 27.72, 19.70, 12.52.

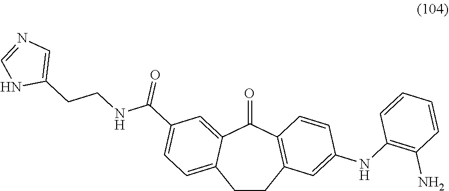 [SMILES: Nc1ccccc1Nc1ccc2c(c1)CCc1ccc(C(=O)NCCc3cnc[nH]3)cc1C2=O]

C₂₇H₂₅N₅O₂ (M=451.20 g/mol)

Masse (ESI_{neg}) (m/z): 450.1 [M−H]⁻

IR (ATR) [cm⁻¹] 2920, 2851, 1602, 1557, 1495, 1353, 1263, 1112, 829, 746, 619

¹H-NMR (400 MHz, DMSO-d6) δ 11.81 (s, 1H), 8.68 (t, J=5.3 Hz, 1H), 8.34-8.30 (m, 1H), 8.04 (s, 1H), 7.98 (d, J=8.8 Hz, 1H), 7.89 (dd, J=7.9, 1.7 Hz, 1H), 7.53 (s, 1H), 7.39 (d, J=8.0 Hz, 1H), 7.02 (d, J=7.6 Hz, 1H), 6.95 (t, J=7.6 Hz, 1H), 6.78 (d, J=7.6 Hz, 1H), 6.64 (dd, J=8.9, 2.1 Hz, 1H), 6.58 (t, J=7.4 Hz, 1H), 6.47 (d, J=1.7 Hz, 1H), 4.86 (s, 2H), 3.49 (d, J=5.3 Hz, 2H), 3.08 (t, J=7.6 Hz, 2H), 3.00 (t, J=7.6 Hz, 2H), 2.76 (s, 2H).

¹³C-NMR (100 MHz, DMSO-d6) δ 190.42, 165.99, 151.43, 145.78, 144.97, 144.13, 139.60, 135.11, 133.98, 133.36, 130.62, 129.66, 129.21, 126.60, 126.34, 126.20, 125.28, 116.95, 116.00, 113.28, 112.31, 79.62, 70.26, 36.17, 34.33.

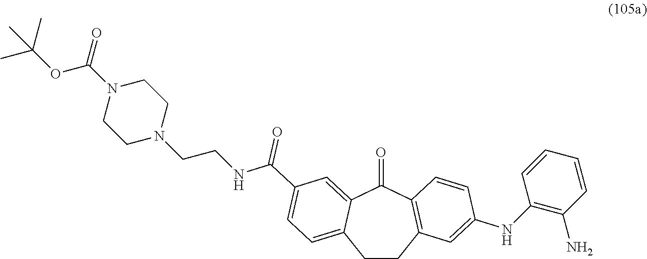 [SMILES: CC(C)(C)OC(=O)N1CCN(CCNC(=O)c2ccc3c(c2)C(=O)c2ccc(Nc4ccccc4N)cc2CC3)CC1]

C₃₃H₃₉N₅O₄ (M=569.30 g/mol)

Masse (ESI_{neg}) (m/z): 568.1 [M−H]⁻

IR (ATR) [cm⁻¹] 3289, 2923, 1660, 1603, 1573, 1417, 1246, 1164, 1113, 860, 786

¹H-NMR (400 MHz, CDCl₃) δ 8.37 (s, 1H), 8.17 (d, J=8.7 Hz, 1H), 8.05-7.93 (m, 2H), 7.31 (d, J=7.0 Hz, 1H), 7.14 (dd, J=17.5, 7.7 Hz, 3H), 6.91-6.76 (m, 3H), 6.68 (dd, J=8.7, 2.1 Hz, 1H), 6.45 (d, J=1.8 Hz, 1H), 5.66 (s, 1H), 3.66 (s, 3H), 3.55 (s, 5H), 3.19-3.13 (m, 2H), 3.12-3.07 (m, 2H), 2.60 (s, 4H), 1.48 (s, 9H).

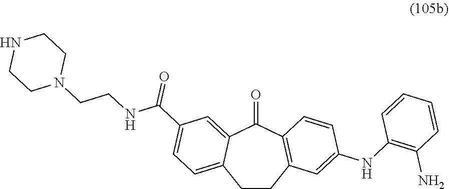 [SMILES: Nc1ccccc1Nc1ccc2c(c1)CCc1ccc(C(=O)NCCN3CCNCC3)cc1C2=O]

C₂₈H₃₁N₅O₂ (M=469.25 g/mol)

Masse (ESIₚₒₛ) (m/z): 470.2 [M+H]⁺

¹H-NMR (400 MHz, CDCl₃) δ 8.30 (s, 1H), 8.12 (d, J=8.8 Hz, 1H), 7.92 (d, J=7.8 Hz, 1H), 7.29 (s, 1H), 7.16-7.06 (m, 2H), 6.85-6.75 (m, 2H), 6.64 (t, J=8.3 Hz, 1H), 6.42 (d, J=5.7 Hz, 1H), 3.55 (d, J=5.2 Hz, 2H), 3.11 (td, J=13.5, 6.6 Hz, 8H), 2.66 (d, J=6.1 Hz, 4H).

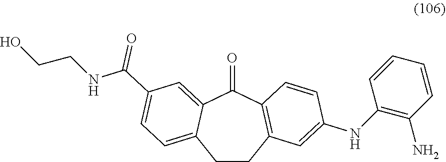 [SMILES: Nc1ccccc1Nc1ccc2c(c1)CCc1ccc(C(=O)NCCO)cc1C2=O]

C₂₄H₂₃N₃O₃ (M=401.17 g/mol)

Masse (ESI_{neg}) (m/z): 400.1 [M−H]⁻

IR (ATR) [cm⁻¹] 3305, 2923, 1603, 1557, 1498, 1354, 1266, 1216, 1113, 1065, 746, 451

¹H-NMR (400 MHz, DMSO-d6) δ 8.53 (s, 1H), 8.33 (s, 1H), 8.01 (s, 1H), 7.98 (d, J=8.8 Hz, 1H), 7.90 (d, J=7.8 Hz, 1H), 7.38 (d, J=7.9 Hz, 1H), 7.02 (d, J=7.7 Hz, 1H), 6.95 (t, J=7.6 Hz, 1H), 6.78 (d, J=8.0 Hz, 1H), 6.63 (d, J=8.9 Hz, 1H), 6.58 (t, J=7.5 Hz, 1H), 6.47 (s, 1H), 4.84 (s, 2H), 4.75 (s, 1H), 3.51 (s, 4H), 3.09 (d, J=8.5 Hz, 2H), 3.00 (s, 2H).

¹³C-NMR (100 MHz, DMSO-d6) δ 190.45, 166.22, 151.43, 145.78, 144.96, 144.13, 139.58, 133.97, 133.29, 130.70, 129.71, 129.17, 126.61, 126.35, 125.26, 116.95, 116.00, 113.28, 112.32, 60.21, 42.68, 36.18, 34.32.

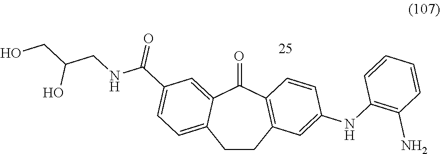 [SMILES: Nc1ccccc1Nc1ccc2c(c1)CCc1ccc(C(=O)NCC(O)CO)cc1C2=O]

C₂₅H₂₅N₃O₄ (M=431.18 g/mol)

Masse (ESI_{neg}) (m/z): 429.7 [M−H]⁻

IR (ATR) [cm⁻¹] 3306, 2920.2851, 1603, 1574, 1557, 1455, 1354, 1263, 1111, 1036, 745

¹H-NMR (400 MHz, DMSO-d6) δ 8.50 (t, J=5.5 Hz, 1H), 8.33 (s, 1H), 8.02 (s, 1H), 7.98 (d, J=8.8 Hz, 1H), 7.91 (d, J=7.7 Hz, 1H), 7.39 (d, J=7.9 Hz, 1H), 7.02 (d, J=7.7 Hz, 1H), 6.95 (t, J=7.6 Hz, 1H), 6.78 (d, J=8.0 Hz, 1H), 6.63 (d, J=8.9 Hz, 1H), 6.59 (t, J=7.5 Hz, 1H), 6.47 (s, 1H), 4.84 (s, 1H), 4.61 (s, 1H), 3.68-3.60 (m, 2H), 3.20 (d, J=6.8 Hz, 1H), 3.09 (d, J=8.9 Hz, 2H), 2.99 (d, J=8.6 Hz, 2H).

¹³C-NMR (100 MHz, DMSO-d6) δ 190.42, 166.49, 151.42, 145.79, 145.02, 144.07, 139.58, 133.97, 133.22, 130.72, 129.73, 129.20, 126.61, 126.35, 125.28, 116.99, 116.02, 113.28, 112.31, 70.85, 64.41, 43.50, 36.16, 34.31.

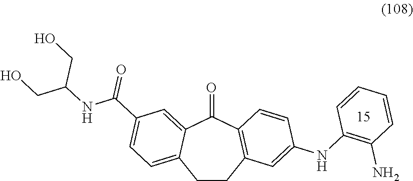 [SMILES: Nc1ccccc1Nc1ccc2c(c1)CCc1ccc(C(=O)NC(CO)CO)cc1C2=O]

C₂₅H₂₅N₃O₄ (M=431.18 g/mol)

Masse (ESI_{neg}) (m/z): 430.3 [M−H]⁻

IR (ATR) [cm⁻¹] 3307, 2918, 2850, 1603, 1574, 1557, 1456, 1353, 1266, 1036, 746, 565

¹H-NMR (400 MHz, DMSO-d6) δ 8.45-8.37 (m, 2H), 7.92 (dd, J=7.9, 1.9 Hz, 1H), 7.28 (dd, J=19.1, 7.1 Hz, 2H), 7.02 (d, J=7.4 Hz, 1H), 6.93 (d, J=7.8 Hz, 1H), 6.78 (t, J=7.4 Hz, 1H), 6.67 (dt, J=6.6, 3.3 Hz, 1H), 6.55-6.50 (m, 1H), 4.35 (d, J=18.9 Hz, 2H), 3.98 (dd, J=11.3, 5.2 Hz, 4H), 3.26 (s, 2H), 3.18-3.11 (m, 1H), 3.09 (d, J=9.6 Hz, 2H), 3.00 (d, J=8.5 Hz, 2H).

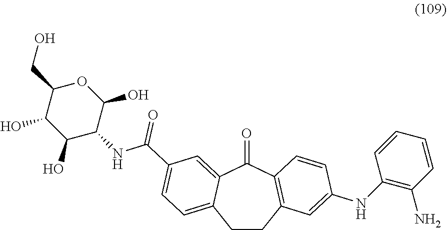 [SMILES: Nc1ccccc1Nc1ccc2c(c1)CCc1ccc(C(=O)N[C@@H]3[C@@H](O)[C@H](O)[C@@H](CO)O[C@H]3O)cc1C2=O]

C₂₈H₂₉N₃O₇ (M=519.20 g/mol)

Masse (ESI_{neg}) (m/z): 518.6 [M−H]⁻

IR (ATR) [cm⁻¹] 3308, 2920, 2851, 1738, 1603, 1557, 1495, 1355, 1262, 1028, 745

¹H-NMR (400 MHz, MeOD-d4) δ 8.09 (s, 1H), 8.17 (s, 1H), 8.01 (d, J=4.1 Hz, 1H), 7.82-7.95 (m, 4H), 7.56 (d, J=9.2 Hz, 1H), 6.50 (s, 1H), 6.32 (d, J=6.2 Hz, 1H), 4.62 (d, J=4.2 Hz, 2H), 4.31 (m, 1H), 4.12 (m, 2H), 3.83 (m, 2H), 3.42-3.58 (m, 2H), 3.14 (m, 1H).

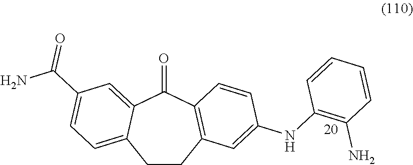 [SMILES: NC(=O)c1ccc2c(c1)C(=O)c1ccc(Nc3ccccc3N)cc1CC2]

C₂₂H₁₉N₃O₂ (M=357.15 g/mol)

Masse (ESI_{neg}) (m/z): 356.0 [M−H]⁻

IR (ATR) [cm⁻¹] 3305, 2922, 2853, 1652, 1602, 1567, 1513, 1353, 1270, 1108, 830, 145, 524

¹H-NMR (400 MHz, DMSO-d6) δ 8.34 (d, J=1.4 Hz, 1H), 8.07 (s, 1H), 8.01 (s, 1H), 7.98 (d, J=8.9 Hz, 1H), 7.92 (dd, J=7.9, 1.4 Hz, 1H), 7.37 (d, J=7.7 Hz, 2H), 7.02 (d, J=7.6 Hz, 1H), 6.95 (t, J=7.6 Hz, 1H), 6.78 (d, J=7.9 Hz, 1H), 6.63 (dd, J=8.9, 2.0 Hz, 1H), 6.60 (d, J=7.5 Hz, 1H), 6.47 (d, J=1.7 Hz, 1H), 4.84 (s, 2H), 3.12-3.05 (m, 2H), 2.99 (d, J=8.7 Hz, 2H), 2.68 (s,

¹³C-NMR (100 MHz, DMSO-d6) δ 190.48, 167.88, 151.41, 145.79, 145.14, 144.11, 139.61, 133.97, 133.01, 130.89, 130.11, 129.16, 126.63, 126.37, 126.22, 125.27, 117.00, 116.01, 113.29, 112.31, 36.15, 34.32.

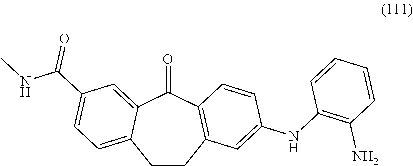 [SMILES: CNC(=O)c1ccc2c(c1)C(=O)c1ccc(Nc3ccccc3N)cc1CC2]

C₂₃H₂₁N₃O₂ (M=371.16 g/mol)

Masse (ESI_{neg}) (m/z): 370.1 [M−H]⁻

IR (ATR) [cm⁻¹] 1633, 1603, 1574, 1455, 1403, 1353, 1262, 1111, 853, 745

¹H-NMR (400 MHz, DMSO-d6) δ 8.53 (d, J=4.3 Hz, 1H), 8.33 (s, 1H), 8.02 (s, 1H), 7.98 (d, J=8.8 Hz, 1H), 7.92-7.84 (m, 1H), 7.38 (d, J=7.9 Hz, 1H), 7.02 (d, J=7.5 Hz, 1H), 6.95 (t, J=7.4 Hz, 1H), 6.78 (d, J=7.7 Hz, 1H), 6.64 (dd, J=8.8, 1.6 Hz, 1H), 6.58 (t, J=7.3 Hz, 1H), 6.47 (s, 1H), 4.86 (s, 2H), 3.09 (d, J=8.2 Hz, 2H), 2.99 (d, J=7.8 Hz, 2H), 2.78 (d, J=4.3 Hz, 3H).

¹³C-NMR (100 MHz, DMSO-d6) δ 189.88, 165.98, 150.93, 145.26, 144.42, 143.65, 139.10, 133.49, 132.76, 130.03, 129.11, 128.74, 126.10, 125.85, 125.73, 124.77, 116.43, 115.50, 112.77, 111.83, 35.66, 33.84, 26.22.

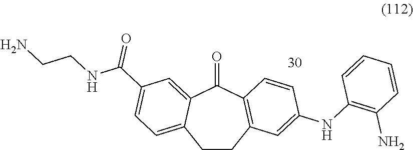 [SMILES: NCCNC(=O)c1ccc2c(c1)C(=O)c1ccc(Nc3ccccc3N)cc1CC2]

C₂₄H₂₄N₄O₂ (M=400.10 g/mol)

Masse (ESI_{neg}) (m/z): 399.1 [M−H]⁻

IR (ATR) [cm⁻¹] 3290, 2920, 2851, 1602, 1564, 1495, 1352, 1262, 1112, 831, 745, 598

¹H-NMR (400 MHz, DMSO-d6) 8.53 (s, 1H), 8.32 (d, J=6.4 Hz, 1H), 8.07 (s, 1H), 8.00-7.94 (m, 1H), 7.91 (d, J=7.7 Hz, 1H), 7.41-7.34 (m, 1H), 7.02 (d, J=7.6 Hz, 1H), 6.95 (t, J=7.5 Hz, 1H), 6.78 (d, J=7.9 Hz, 1H), 6.64 (d, J=8.9 Hz, 1H), 6.58 (t, J=7.5 Hz, 1H), 6.46 (d, J=13.8 Hz, 1H), 4.87 (s, 2H), 3.50 (s, 2H), 3.26 (d, J=5.7 Hz, 2H), 3.08 (s, 2H), 3.00 (s, 2H).

¹³C-NMR (100 MHz, DMSO-d6) δ 190.45, 166.22, 151.43, 145.77, 144.89, 144.12, 139.60, 133.97, 133.41, 130.70, 129.71, 129.14, 126.57, 126.31, 126.21, 125.29, 116.93, 116.01, 113.28, 112.31, 79.63, 70.26, 36.19, 34.33.

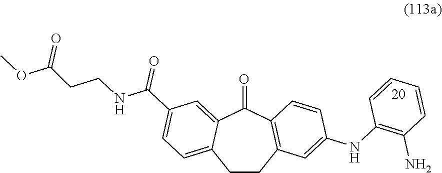 [SMILES: COC(=O)CCNC(=O)c1ccc2c(c1)C(=O)c1ccc(Nc3ccccc3N)cc1CC2]

C₂₆H₂₅N₃O₄ (M=443.18 g/mol)

Masse (ESIₚₒₛ) (m/z): 442.1 [M−H]⁻

¹H-NMR (400 MHz, CDCl₃) δ 8.56 (s, 1H), 8.27 (d, J=15.5 Hz, 1H), 8.21 (d, J=7.8 Hz, 1H), 7.97 (s, 2H), 7.57-7.49 (m, 2H), 7.14 (s, 3H), 6.36 (s, 1H), 3.74 (s, 3H), 3.28 (s, 2H), 2.96 (s, 2H), 0.87 (d, J=7.4 Hz, 4H).

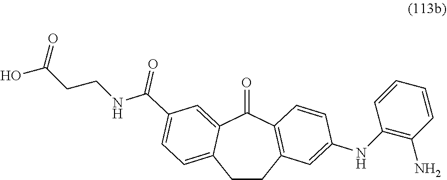 [SMILES: Nc1ccccc1Nc1ccc2c(c1)CCc1ccc(C(=O)NCCC(=O)O)cc1C2=O]

C₂₅H₂₃N₃O₄ (M=429.17 g/mol)

Masse (ESIₚₒₛ) (m/z): 428.4 [M−H]⁻

¹H-NMR (400 MHz, DMSO-d6) δ 8.68 (s, 2H), 7.42 (d, J=46.0 Hz, 3H), 6.98 (d, J=34.9 Hz, 2H), 6.66-6.42 (m, 2H), 4.86 (s, 2H), 3.30 (s, 2H), 3.16 (s, 1H), 3.08 (s, 2H), 2.99 (s, 2H), 2.34 (s, 2H).

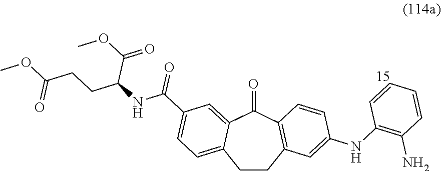 [SMILES: COC(=O)CC[C@H](NC(=O)c1ccc2c(c1)C(=O)c1ccc(Nc3ccccc3N)cc1CC2)C(=O)OC]

C₂₉H₂₉N₃O₆ (M=515.21 g/mol)

Masse (ESI_{neg}) (m/z): 514.1 [M−H]⁻

IR (ATR) [cm⁻¹] 2921, 2852, 1732, 1606, 1580, 1505, 1447, 1356, 1308, 1208, 1105, 848, 744

¹H-NMR (400 MHz, DMSO-d6) δ 8.89 (d, J=7.4 Hz, 1H), 8.36 (d, J=1.8 Hz, 1H), 8.02 (s, 1H), 7.98 (d, J=8.9 Hz, 1H), 7.92 (dd, J=7.9, 1.9 Hz, 1H), 7.41 (d, J=8.0 Hz, 1H), 7.04-7.00 (m, 1H), 6.98-6.92 (m, 1H), 6.78 (dd, J=8.0, 1.1 Hz, 1H), 6.64 (dd, J=8.9, 2.2 Hz, 1H), 6.61-6.56 (m, 1H), 6.47 (d, J=2.0 Hz, 1H), 4.84 (s, 2H), 4.47 (ddd, J=9.5, 7.4, 5.4 Hz, 1H), 3.64 (s, 3H), 3.58 (s, 3H), 3.14-3.06 (m, 2H), 3.00 (d, J=9.2 Hz, 2H), 2.44 (t, J=7.5 Hz, 2H), 2.16-1.96 (m, 2H).

¹³C NMR (100 MHz, DMSO-d6) δ 189.93, 172.65, 172.13, 166.06, 150.97, 145.31, 144.96, 143.61, 139.20, 133.45, 131.82, 130.48, 129.39, 128.74, 126.14, 125.87, 125.63, 124.74, 116.48, 115.51, 112.79, 111.82, 51.92, 51.33, 35.66, 33.82, 29.93, 25.63.

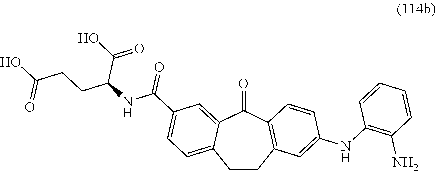 [SMILES: Nc1ccccc1Nc1ccc2c(c1)CCc1ccc(C(=O)N[C@@H](CCC(=O)O)C(=O)O)cc1C2=O]

C₂₇H₂₅N₃O₆ (M=487.17 g/mol)

Masse (ESI_{neg}) (m/z): 486.1 [M−H]⁻

IR (ATR) [cm⁻¹] 2922, 2852, 1714, 1603, 1519, 1451, 1394, 1266, 1101, 743

¹H-NMR (400 MHz, DMSO-d6) δ 8.75 (d, J=7.7 Hz, 1H), 8.38 (d, J=1.8 Hz, 1H), 8.03 (s, 1H), 7.99 (dd, J=8.6, 5.0 Hz, 2H), 7.94 (dd, J=7.9, 1.9 Hz, 1H), 7.72 (d, J=8.4 Hz, 1H), 7.58-7.49 (m, 1H), 7.42 (dd, J=10.0, 4.3 Hz, 2H), 7.07-7.00 (m, 1H), 7.01-6.93 (m, 1H), 6.81 (dd, J=7.9, 1.1 Hz, 1H), 6.70-6.56 (m, 2H), 6.48 (d, J=2.0 Hz, 1H), 4.41 (ddd, J=10.0, 7.7, 4.9 Hz, 1H), 4.02 (dd, J=14.2, 7.1 Hz, 1H), 3.10 (d, J=9.0 Hz, 2H), 3.00 (d, J=8.7 Hz, 2H), 2.35 (t, J=7.4 Hz, 2H), 2.09 (dt, J=10.2, 6.8 Hz, 1H), 1.94 (d, J=30.2 Hz, 2H), 1.18 (dd, J=18.7, 11.5 Hz, 2H).

¹³C-NMR (100 MHz, DMSO-d6) δ 190.48, 174.28, 173.78, 166.47, 151.38, 145.80, 145.29, 139.67, 133.96, 132.66, 130.96, 129.90, 129.19, 128.29, 127.83, 126.60, 126.34, 126.25, 125.60, 125.02, 119.57, 117.42, 116.33, 113.36, 112.37, 110.05, 52.44, 36.17, 34.33, 30.88, 26.35.

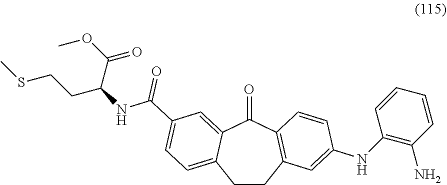 [SMILES: COC(=O)[C@H](CCSC)NC(=O)c1ccc2c(c1)C(=O)c1ccc(Nc3ccccc3N)cc1CC2]

C₂₈H₂₉N₃O₄S (M=503.19 g/mol)

Masse (ESI_{neg}) (m/z): 502.1 [M−H]⁻

IR (ATR) [cm⁻¹] 2920, 2852, 1737, 1604, 1575, 1504, 1444, 1353, 1256, 1209, 742

¹H-NMR (400 MHz, CDCl₃) δ 8.43 (s, 1H), 8.31 (s, 1H), 8.16 (s, 1H), 8.01 (s, 1H), 7.88 (s, 2H), 7.46 (s, 4H), 7.12 (s, 3H), 6.73-6.40 (m, 2H), 4.92 (s, 2H), 3.80 (s, 2H), 3.25 (s, 2H), 2.96 (s, 2H), 2.88 (s, 2H), 2.12 (d, J=7.5 Hz, 3H), 2.10 (s, 2H).

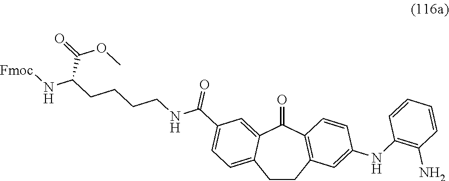

Compound 116a was not further characterized but applied directly to the next step

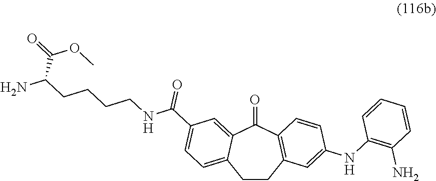 [SMILES: COC(=O)[C@@H](N)CCCCNC(=O)c1ccc2c(c1)C(=O)c1ccc(Nc3ccccc3N)cc1CC2]

C₂₉H₃₂N₄O₄ (M=500.24 g/mol)

Masse (ESI_{neg}) (m/z): 499.1 [M−H]⁻

IR (ATR) [cm⁻¹] 3294, 2922, 2852, 1731, 1633, 1574, 1454, 1353, 1261, 1214, 1112, 746

¹H-NMR (400 MHz, DMSO-d6) δ 8.55 (s, 1H), 8.32 (s, 1H), 8.02 (s, 1H), 7.98 (d, J=8.9 Hz, 1H), 7.87 (t, J=9.6 Hz, 1H), 7.38 (d, J=7.9 Hz, 1H), 7.02 (d, J=7.6 Hz, 1H), 6.95 (t, J=7.6 Hz, 1H), 6.78 (d, J=8.0 Hz, 1H), 6.64 (d, J=8.8 Hz, 1H), 6.59 (t, J=7.5 Hz, 1H), 6.45 (d, J=15.0 Hz, 1H), 4.85 (s, 2H), 3.61 (s, 2H), 3.24 (d, J=5.6 Hz, 3H), 3.09 (d, J=4.2 Hz, 2H), 3.00 (s, 2H), 1.58 (d, J=5.8 Hz, 2H), 1.49 (d, J=7.0 Hz, 6H), 1.39-1.26 (m, 4H).

¹³C-NMR (100 MHz, DMSO-d6) δ 190.46, 176.41, 165.96, 151.41, 145.76, 144.87, 144.13, 139.59, 133.83, 133.61, 129.62, 129.15, 126.60, 126.34, 125.25, 116.94, 115.99, 113.28, 112.31, 54.27, 51.88, 34.52, 34.32, 29.31.

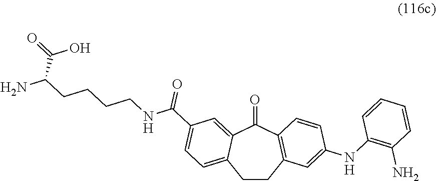 [SMILES: Nc1ccccc1Nc1ccc2c(c1)CCc1ccc(C(=O)NCCCC[C@H](N)C(=O)O)cc1C2=O]

C₂₈H₃₀N₄O₄ (M=486.23 g/mol)

Masse (ESIₚₒₛ) (m/z): 509.3 [M+Na]⁺

IR (ATR) [cm⁻¹] 2921, 2852, 1693, 1603, 1392, 1226, 1152, 1035, 929, 729, 498

¹H-NMR (400 MHz, DMSO-d6) δ 8.66 (d, J=27.0 Hz, 1H), 8.28 (d, J=32.2 Hz, 2H), 7.93 (dd, J=32.3, 16.6 Hz, 2H), 7.37 (d, J=6.9 Hz, 1H), 6.97 (dd, J=41.4, 11.4 Hz, 2H), 6.77 (d, J=7.7 Hz, 1H), 6.62 (dd, J=28.0, 7.6 Hz, 2H), 6.48 (d, J=11.3 Hz, 1H), 4.91 (s, 2H), 3.23 (s, 2H), 3.07 (s, 2H), 2.99 (s, 2H), 1.43 (d, J=45.6 Hz, 6H), 1.32 (d, J=29.2 Hz, 2H), 1.23 (s, 4H).

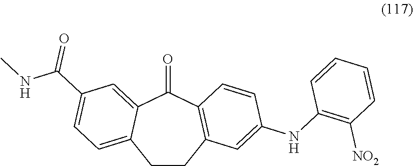 [SMILES: CNC(=O)c1ccc2c(c1)C(=O)c1ccc(Nc3ccccc3[N+](=O)[O-])cc1CC2]

C₂₃H₁₉N₃O₄ (M=401.14 g/mol)

Masse (ESI_{neg}) (m/z): 400.3 [M−H]⁻

¹H-NMR (400 MHz, DMSO-d6) δ 9.32 (s, 1H), 8.56 (d, J=4.2 Hz, 1H), 8.35 (s, 1H), 8.11 (d, J=8.2 Hz, 1H), 8.02 (d, J=8.6 Hz, 1H), 7.94 (d, J=7.4 Hz, 1H), 7.63 (t, J=7.6 Hz, 1H), 7.56 (d, J=8.2 Hz, 1H), 7.44 (d, J=7.9 Hz, 1H), 7.19 (t, J=11.1 Hz, 1H), 7.17-7.09 (m, 2H), 3.17 (t, J=7.6 Hz, 2H), 3.13 (d, J=8.1 Hz, 2H), 2.79 (d, J=4.2 Hz, 3H).

¹³C-NMR (100 MHz, DMSO-d6) δ 191.48, 165.82, 145.28, 144.71, 144.56, 138.42, 137.89, 137.64, 135.39, 132.91, 132.71, 130.93, 130.55, 129.25, 128.98, 126.11, 121.21, 120.64, 119.14, 117.02, 34.76, 33.72, 26.22.

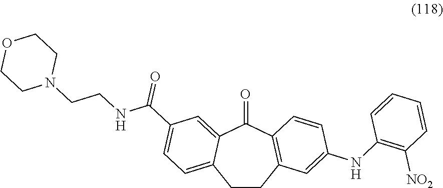 [SMILES: O=C(NCCN1CCOCC1)c1ccc2c(c1)C(=O)c1ccc(Nc3ccccc3[N+](=O)[O-])cc1CC2]

C₂₈H₂₈N₄O₅ (M=500.21 g/mol)

Masse (ESI_{neg}) (m/z): 498.9 [M−H]⁻

IR (ATR) [cm⁻¹] 2919, 2854, 1630, 1600, 1505, 1445, 1346, 1146, 933, 644, 515

¹H-NMR (200 MHz, CDCl₃) δ 9.52 (s, 1H), 8.46 (s, 1H), 8.30-8.20 (m, 2H), 8.07 (d, J=6.4 Hz, 1H), 7.55 (s, 2H), 7.39 (d, J=8.0 Hz, 1H), 7.14 (s, 1H), 7.00 (d, J=8.7 Hz, 1H), 3.78 (d, J=38.1 Hz, 6H), 3.27 (s, 4H), 2.80 (s, 2H), 2.72 (s, 4H).

¹³C-NMR (50 MHz, CDCl₃) δ 191.99, 166.46, 145.04, 144.56, 143.43, 140.18, 138.36, 135.53, 133.46, 126.73, 121.52, 119.41, 119.17, 117.36, 66.08, 57.11, 53.24, 35.54, 35.12.

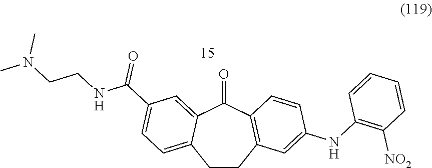 [SMILES: CN(C)CCNC(=O)c1ccc2c(c1)C(=O)c1ccc(Nc3ccccc3[N+](=O)[O-])cc1CC2]

C₂₆H₂₆N₄O₄ (M=458.20 g/mol)

Masse (ESI_{neg}) (m/z): 456.9 [M−H]⁻

IR (ATR) [cm⁻¹] 2921, 2852, 1640, 1575, 1497, 1344, 1445, 1147, 1040, 852, 737

¹H-NMR (200 MHz, CDCl₃) δ 9.51 (s, 1H), 8.42 (s, 1H), 8.24 (t, J=8.2 Hz, 2H), 8.04 (d, J=5.9 Hz, 1H), 7.53 (d, J=3.2 Hz, 2H), 7.13 (s, 2H), 6.98 (d, J=5.3 Hz, 1H), 3.61 (d, J=5.4 Hz, 2H), 3.26 (s, 4H), 2.63 (t, J=6.0 Hz, 2H), 2.36 (s, 6H).

¹³C-NMR (50 MHz, CDCl₃) δ 192.42, 166.57, 144.90, 144.64, 143.37, 140.23, 138.47, 135.48, 133.54, 133.11, 131.58, 129.46, 128.87, 126.83, 121.49, 119.45, 119.12, 117.44, 57.89, 45.14, 37.13, 35.20, 34.66.

### Biological Activity

The substances can be tested by mixing them with immune cells subject to stimulus and measuring the resulting inflammatory response. A standard assay involves the application of lipopolysaccharide (e.g. 10 μg/mL) to human blood derived immune cells and then detecting the concentration of cytokines (IL-6, TNFa) after 24 h. When tested in this way, the described compounds have the following effects as recorded in Table 2.

Amount of cytokine produced as % control

| | Compound | | | | | | | | | |
|---|---|---|---|---|---|---|---|---|---|---|
| | | | IL-6 | IL-6 | IL-6 | TNFa | IL-6 | IL-6 | TNFa | TNFa |
| | IL_6 | TNFa | hBuffy | hBuffy | hBuffy | hBuffy | BL6 | BL6 | BL6 | BL6 |
| | Spleen | Spleen | Coat | Coat | Coat | Coat | Blood | Blood | Blood | Blood |
| | (100 ng | (100 ng | (100 ng | (100 ng | (100 ng | (100 ng | (500 ng | (500 ng | (500 ng | (500 ng |
| | LPS) | LPS) | LPS) | LPS) | LPS) | LPS) | LPS) | LPS) | LPS) | LPS) |
| Number | 10 nM | 10 nM | 1 nM | 10 nM | 100 nM | 1 nM | 10 nM | 100 nM | 10 nM | 100 nM |
| 124 | 36 | 16 | 100 | 70 | 55 | 74 | | | | |
| 303 | 39 | 33 | 89 | 65 | 29 | 84 | | | | |
| 398 | 16 | 24 | 93 | 78 | 42 | 71 | | | | |
| 430 | 14 | 11 | 95 | 90 | 92 | 10 | 60 | 0 | 6 | 0 |
| 487 | 24 | 13 | 36 | 18 | 13 | 17 | 64 | 37 | 12 | 1 |
| 127 | 24 | 8 | 67 | 24 | 34 | 22 | | | | |
| 307 | 9 | 37 | 86 | 52 | 30 | 55 | | | | |
| 403 | 14 | 14 | 106 | 49 | 28 | 41 | | | | |
| 434 | 22 | 10 | 76 | 49 | 21 | 46 | | | | |
| 511 | 10 | 4 | 39 | 11 | 6 | 18 | 65 | 21 | 7 | 0 |
| 157 | 19 | 33 | 87 | 74 | 57 | 83 | | | | |
| 308 | 17 | 37 | 66 | 28 | 19 | 56 | | | | |
| 404 | 48 | 49 | 95 | 83 | 26 | 85 | | | | |
| 441 | 11 | 8 | 65 | 26 | 20 | 18 | 34 | 26 | 0 | 1 |
| 520 | 9 | 14 | 23 | 14 | 11 | 2 | 61 | 60 | 17 | 2 |
| 211 | 28 | 16 | 70 | 18 | 18 | 72 | | | | |
| 340 | 7 | 9 | 60 | 40 | 27 | 56 | | | | |
| 407 | 42 | 48 | 56 | 33 | 38 | 21 | 57 | 46 | 15 | 1 |
| 463 | 9 | 5 | 31 | 26 | 13 | 34 | | | | |
| 521 | 9 | 7 | 19 | 13 | 6 | 0 | 64 | 17 | 10 | 0 |
| 229 | 20 | 28 | 68 | 32 | 24 | 42 | | | | |
| 352 | 8 | 6 | 34 | 15 | 15 | 23 | | | | |
| 410 | 20 | 9 | 25 | 12 | 12 | 4 | 44 | 6 | 4 | 0 |
| 472 | 13 | 15 | 43 | 24 | 15 | 57 | | | | |
| 532 | 13 | 7 | 87 | 104 | 69 | 41 | | | | |
| 231 | 9 | 15 | 76 | 27 | 22 | 45 | | | | |
| 353 | 10 | 4 | 51 | 17 | 20 | 14 | 32 | 16 | 7 | 0 |
| 411 | 8 | 5 | 35 | 13 | 8 | 18 | | | | |
| 481 | 152 | 67 | 89 | 76 | 70 | 71 | | | | |
| 367 | 10 | 19 | 95 | 68 | 61 | 62 | | | | |
| 232 | 27 | 37 | 55 | 25 | 17 | 59 | | | | |
| 385 | 0 | 11 | 50 | 14 | 12 | 22 | 59 | 21 | 12 | 0 |
| 412 | 8 | 6 | 87 | 25 | 30 | 59 | | | | |
| 484 | 10 | 6 | 36 | 11 | 15 | 29 | | | | |
| 274 | 24 | 28 | 87 | 40 | 26 | 72 | | | | |
| 392 | 12 | 20 | 78 | 87 | 44 | 84 | | | | |
| 418 | 21 | 31 | 66 | 34 | 19 | 16 | 29 | 25 | 3 | 0 |
| 485 | 20 | 11 | 27 | 30 | 14 | 0 | 63 | 9 | 5 | 0 |
| CSY3697 | 33 | 29 | 69 | | | 62 | | 32 | | 0 |
| SB203580 | 10 | 11 | 61 | | | 85 | | 22 | | 4 |

Example—uptake into the mouse brain.

Compounds can be assessed for central uptake by administration to a suitable experimental system such as a laboratory mouse. In this case, substances were formulated in a 1% hydroxypropylcellulose vehicle and administered p.o. at 0.4 mg/kg. Samples were taken at 2 and 4 h and concentration tested by HPLC MSMS vs. Standards for each substance.

Levels detected in the brain are reported in Table 3 (all values nM).

Matrix

| | Brain | Brain |
|---|---|---|
| Time | 2 h | 4 h |
| 434 | 26 | 15 |
| 274 | 9 | 7 |
| 412 | 10 | 3 |
| 303 | 7 | 1 |
| 485 | 5 | 2 |
| 157_1 | 6 | 3 |
| 441 | 27 | 14 |
| 463 | 48 | 31 |
| 340 | 12 | 4 |
| 403 | 13 | 4 |
| 231 | 21 | 6 |
| 232 | 20 | 5 |
| 430 | 9 | 4 |
| 211 | 12 | 4 |
| 308 | 8 | 4 |
| 418 | 7 | 4 |
| 411_1 | 8 | 5 |
| 410 | 1 | 1 |
| 385 | 25 | 23 |
| 367 | 1 | 0 |
| 484 | 10 | 6 |
| 352 | 85 | 16 |
| CSY 3697 | 2 | 2 |
| 472 | 438 | 505 |
| 398 | 5 | 1 |
| 404 | 8 | 3 |
| 407 | 6 | 2 |
| 127 | 5 | 2 |
| CSY5530 | 7 | 3 |
| 307 | 7 | 5 |
| 353 | 1 | 1 |
| 521 | 11 | 7 |
| 392 | 1 | 0 |
| 481 | 18 | 9 |
| 487 | 5 | 3 |
| 520 | 109 | 46 |

### Other Embodiments

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### Abbreviations

The following abbreviations were used as noted:

MeOH: methanol

NaHCO₃: sodium bicarbonate

K₂CO₃: potassium carbonate

MS: mass spectrometry

DMSO: dimethyl sulfoxide

TLC: thinlayer chromatography

Et₃N: triethylamin

EtOAc: ethyl acetate

DCM: dichloromethane

NH₄Cl: ammonium chloride

THF: tetrahydrofuran

Na₂CO₃: sodium carbonate

EDCI: N-Ethyl-N′-(3-dimethylaminopropyl)carbodiimide hydrochloride

DMAP: 4-dimethylamino pyridine

### CITATION LIST PATENT LITERATURE

### Citations—Non Patent Literature

Koeberle S C, Romir J, Fischer S, Koeberle A, Schattel V, Albrecht W, Grütter C, Werz O, Rauh D, Stehle T, Laufer S A. Nat Chem Biol. 2011 Dec. 25; 8(2):141-3. doi: 10.1038/nchembio.761. Skepinone-L is a selective p38 mitogen-activated protein kinase inhibitor.

What is claimed is:

1. A compound with general structure (Formula 1) 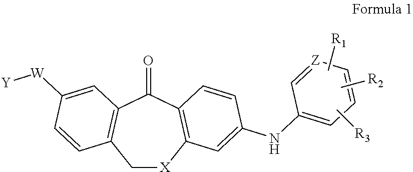
or a pharmaceutically acceptable salt, solvate, or hydrate thereof;
W=bond*, —C(═O)—;
X=O, CH₂;
Y=OR₄, NR₉R₄;
Z=N, C(R₁);
R₁, R₂, R₃=independent of each other H, F, Cl, Br, I, NH₂, NHCH₃, N(CH₃)₂, NO₂;
R₄=H, OH, phenyl, C₁-C₁₀-alkyl, linear or cyclic, branched or unbranched; optionally substituted with 1 to 6 substituents of the group: F, OH, OR₆, SH, SCH₃, NH₂, NHR₆, NR₆R₇, COOH, COOCH₃, 1-Morpholinyl, 1-piperidinyl, 1-(4-R₈)piperazinyl, 3-(1H)indolyl, 4-(1H)imidazolyl, phenyl (optionally substituted with OH, OCH₃, F, Cl, Br, I, N(R₉)₂);
or C₁-C₈-alkyl as described above and 1 or 2 links of the carbon chain replaced by O, NH, NR₆;
or 2-(2-oxa-6-azaspiro[3,3]heptan-6-yl)ethyl;
R₆, R₇=independent of each other C₁-C₂-alkyl, optionally substituted with OH, OCH₃, NH₂, NHCH₃, N(CH₃)₂, COOH, COOCH₃, 1-morpholinyl, 1-piperidinyl, 1-(4-R₈)piperazinyl, phenyl;
R₈=H, CH₃, Boc, Fmoc, Z;
R₉=H, Me;
or R₄, R₉=—CH₂—(V)ₙ—CH₂—, V=CH₂, S, O; n=1-4; and
* “bond” indicates the direct connection between Y and the aromatic ring.

2. A use of a compound of claim 1, or a pharmaceutically acceptable salt, solvate, or hydrate thereof, to treat a degenerative disease of the brain.

3. A pharmaceutical composition comprising a compound of claim 1, or a pharmaceutically acceptable salt, solvate, or hydrate thereof, and a pharmaceutically acceptable carrier.

4. A method of treating a disease, disorder, or symptom thereof in a subject comprising administering to the subject a compound of claim 1, or a pharmaceutically acceptable salt, solvate, or hydrate thereof.

5. The method of claim 4, wherein the disease or disorder is a degenerative disease of the brain.
